SEQUENCE LISTING (384 sequences)

SEQ ID NO: 1 - DNA, 927 bp, Escherichia coli
atgatgaaaatagagccttcaattttgccttctcttgcctggtttgcgctgattgttcgtgccggcagtttttccagagccgtttctgaaatgggtattacgctggccgcattgtcccagaatttaaaatctcttgaggaacgtcttaacaccaagctgatataccgtacgacccgcaatatctcgcttactgaggaaggacagcgtctgtatgaagtgctggtgtcggctttggggcaaattgatgatgctttgaaagatgttggtgatacccaacttgaacctacaggcctgttgagaataaattcctcccgtgttgcagcacggatgcttgttgagcctcatattggcgagtttcttactcgttaccccaaaacaaagatagaactcattatggatgacggtttatctaacattattgcggacggctgtgatgtgggcatacgtcttgagcaaggactggatgagcatatgactgccgttcctgtttctccgcttattaaactggttactgtggcatcgccagattatttaaaagaacacggtattcctgaaacgcctcaggaactgagtaaccataattgccttcggttgcggcataaaagtagcggtgcactctctgcatgggagttttctaaggttgtggggggtaatgaagagttcgaaatagaggtttcaggtaagtatatttctaatgatgacgaaagcatgatacgaatggcgctgaatggaacgggtatcattcaacatctggattttgcaatcgctgaacatatcaatgcgggcaagttacagccgattcttgaggattgggctgtttcatttcctgggttttatatttatgtttcatcacgggtgagaatgccctctaaagttcgtgcttttattgattttatggtggagaaaagagtaaaactcgaaaattga

SEQ ID NO: 2 - DNA, 1872 bp, Escherichia coli
atgatggacagcttacgcacggctgcaaacagtctcgtgctcaagattattttcggtatcattatcgtgtcgttcatattgaccggcgtgagtggttacctgattggcggaggcaataactacgccgcaaaagtgaatgaccaggaaatcagccgtgggcaattcgagaatgccttcaacagcgagcgtaatcgcatgcagcaacagctgggcgatcaatactccgagctggcagcgaacgaaggctatatgaaaaccctgcgtcaacaggtgctgaatcgtctgatcgacgaggcgctgctggatcagtacgcacgtgagctgaaactgggtatcagcgatgagcaggttaaacaggcgattttcgcgaccccagccttccaggttgatggcaaatttgataacagccgctataacggtatcctcaaccagatggggatgaccgccgatcagtacgcccaggcgctgcgtaaccagctcactacccaacagctgattaacggcgttgctggtaccgattttatgctgaaaggtgaaaccgacgagctggcggcactggtcgctcaacaacgcgtggtgcgtgaagcgactatcgatgttaacgcgctggcggcgaagcagcctgtgaccgaacaggaaattgccagctactacgaacaaaacaaaaacaatttcatgacgccggaacaattccgcgtgagttacatcaagctggatgccgcaacgatgcagcaaccggttagcgatgcggatctccagagctactacgatcagcatcaggatcaattcacccagccgcagcgtacccgttacagcatcatccagaccaaaactgaagatgaagcgaaagcggtacttgatgagctgaataaaggcggtgattttgctgcattagccaaagaaaaatctgccgatattatctctgctcgtaacggcggcgatatgggttggttagaagatgccactatcccggatgaactgaaaaatgctggtctgaaagaaaaaggccaactgtctggtgtcatcaaatcttcggtcggtttcctgattgtacgtctggacgacattcagccagcgaaagtgaaatcgttagacgaagtacgtgacgatgtcgcggcgaaagtgaaacacgaaaaagccctcgatgcgtactacgcgctgcagcagaaagtgagcgatgcggcaagcaacgacaccgagtctctggccggtgcagagcaagctgccggcgttaaagccactcagacgggttggttcagcaaagagaacctgccggaagagttgaacttcaagccggttgctgacgctatctttaacggcggtctggtaggtgaaaacggcgcgccgggcatcaactctgacatcatcaccgtagacggcgaccgcgcattcgtgctgcgcatcagcgagcacaaaccggaagcggtgaaaccgttggcagatgttcaggaacaagttaaggcactggttcagcacaacaaagctgaacaacaggcgaaagtggatgctgagaaactgctggttgatttgaaagccggcaaaggtgcggaagctatgcaggctgccggtctgaaatttggcgagccgaaaaccttaagccgttccggtcgtgacccgattagccaggcggcgtttgcactgccactgccagcgaaagacaaaccgagctacggtatggcgaccgatatgcaaggtaatgtggttctgctggcgctggatgaagtgaaacaaggttcaatgccggaagatcagaaaaaagcgatggtgcagggtatcacccagaacaacgcacaaatcgtctttgaagctctgatgagtaacctgcgtaaagaggcgaaaatcaaaattggcgatgcgttggaacagcaataa

SEQ ID NO: 3 - DNA, 1362 bp, Escherichia coli
atgaagacgttatctcccgctgtgattactttaccctggcgtcaggacgccgctgaattttatttctcccgcttaagccacctgccgtgggcgatgcttttacactccggctatgccgatcatccgtatagccgctttgatattgtggtcgccgatccgatttgcactttaaccactttaggtaaagaaaccgttgttagtgaaagcgaaaaacgcacaacgaccactgatgacccgctacaggtgctccagcaggtgctggatcgcgcagacattcgcccaacgcataacgaagatttgccatttcagggcggcgcactggggttgtttggctacgatctgggccgccgttttgagtcactgccagaaattgcggaacaagatatcgttctgccggatatggcagtgggtatctacgattgggcgctcattgtcgaccaccagcgtcatacagtttctttgctgagtcataatgatgtcaatgcccgtcgggcctggctggaaagccagcaattctcaccgcaggaagatttcacgctcacttccgactggcaatccaatatgacccgcgagcagtacggcgaaaaatttcgccgggtacaggaatatctgcacagcggtgattgctatcaggtgaatctcgcccagcgttttcatgcgacctattctggcgatgaatggcaggcattccttcagcttaatcaggccaaccgcgcgccatttagcgcttttttacgtcttgaacagggtgcaattttaagcctttcgccagagcggtttattctttgtgataatagtgaaatccagacccgcccgattaaaggcacgctaccacgcctgcccgctcctcaggaagatagcaaacaagcagaaaaactggcgaactcagcgaaagatcgtgccgaaaatctgatgattgtcgatttaatgcgtaatgatatcggtcgtgttgccgtagcaggttcggtaaaagtaccagagctgttcgtggtggaacccttccctgccgtgcatcatctggtcagcaccataacggcgcaactaccagaacagttacactccagcgatctgctgcgcgcagcttttcctggtggctcaataaccggggctccgaaagtacgggctatggaaattatcgacgaactggaaccgcagcgacgtaatgcctggtgcggcagcattggctatttgagcttttgcggcaacatggataccagcattactatccgcacgctgactgccattaacggacaaatatactgctctgcgggaggtggaattgtcgccgatagccaggaagaagcggaatatcaggaaacttttgataaagttaataagatattacgccaactggagaagtaa

SEQ ID NO: 4 - DNA, 3036 bp, Escherichia coli
atgcaaaggaaaaaattattgtctgtttgtgttgccatggcgttaagtagccagacatgggctgcggatacttcaatacccgactcaaccgaaaaaacacgtaagtccagtaagataacatgtccggcaaatattcattccctgagcaaagagcagctagaaaatttatccgcggagtgcagagaaaataaagatagtgcagttttaccatgggcagctgcaggtcttgctgcagtagctacgggaattgctatatataccttaagtgacgatgacaatcaccaccataataactccccagttccggatgatggcggtgatacgcctgttccgccagacgacggcggtgatacgccagttccggatgatggcggtgatacgcctgttccgccagacgacggcggtgatacgccagttccgcctgatgacggcggtgatacgccggttccgccagatgacggcggtgatacgccagttccgccagacgatggtggcgataccccggttaaacacaatcctgtcgtctataaaaatggcgtaacttgggatcaggatgcaaaaactgtcaaaattcgcgaaactacctttacctacagccaaaatgccgatgggagctacaccttaacggcgcctgagggcaaggagacgatagttaagtcctggacagtccatgacgacagcaacaccgttgtctttgatggcgtaaatacctcaggtggcatcacctggagttacgacgatgatggactcatccacatcactaaagaggccggcgtcgttgttgacggcacaaccgggaatgcgattgagtttggtaacgcgatcatcaccgatcagggcggcaacacggcgctgaatggcggtactgtcatgacggttgatggtgacaacatctccctcaacaacgacggcaaaacgacggccatcggagaaggctcggttgtcggtatactcactggcgacaatatcaccattaacaataatggcgagacggaagttgatggcggtaccgcggtaatcatcaatggcgataataccaagctaaacactgcgggcgactcgaccatcaccaatggtggtacgggtagccttatcaatggcgataatgcgcgggtcgataatcaaggtaccatgagcgttgacggagagaattccactggctcgaaaattgtcggtgacggtgccaccatcaaacaggaaggcgacttatacgtaagcggtggtgcgcacggtattgacgttgacggcaatgatacgttcgttagcaacaaaggtaatattacggtcatcgaagataactctattggcatgctgcttgatggtgatggcgtcagcgtgatcaacatgggcgacctcaatgttggtcaggccgcagcgggtgaaaatgccattggcattcagatcgacggtgataatgcgacatttgttaacgttggtgatatttccgcgaccaatgccggaaccggtgtcagcgttgctggggataaagctaatatttcactcgcgggtgggttagacgttggggatttctccaccggtcttgatgtctctggcaataataacaacatgacgctggcgacttatgagctgaatgtgacagggcaaaaagcgacgggtgttaatgtttcgggtgatggtaatactatcgaaatagcgggcagtattctggttgataaagatcagaaagccgataatgcgcagccttacttctttaatccctcaaccggggtaaatatcagcggtgacaacaacgacgtcacgctggatggtcagttaaccgttgttgctgatagtaaaaccacgtcgcgttcctatgcgagttacgacggtgctcaggaacacattgcaggtattgttattgctggcgatgacaatacctttacgcttaatggcggtgtccattttgtcggtgaaaaaaatgtcatggatgatggctcaaaaccatcagcttctcgacgtggtatcggagacacaccgcttattaacgttgatgggcattccccggtctacctgaatggtgagtcaaccatcagcggcgagttcccgctggggttcgaaaatctcattcagttgagccatggcgcggagctggagattggcgctgacgcaacgtttgatatgagtgatgtggattctttcacttattattatcgtgtagcgttatccaccattagtattgattctggtgcgaaggcgacaaacaacggtgaagtggaactgaaaaatatcggttttgcggctgcctggaataaagattcaacggtaatcaataacggatccattggcctggcgatgtacgattttggtaccgacccggcaccaaaagtctttgatgttgaatacggtgggattggcgttaacaatggtaccatgaccaccaaaatgatgaatcagcacagtgtgctgaattatccggcagagtggaacttgtctgatggtacctctttcaataacaaggcattagggctgacaggtatgcttgccagctactcgtcatccattctgaacggcgaaacgggtattattgatatgtatggccgtggcagtgtagggatgctagccatcgataaatcaaccgcggataacgaagggcaaatcaccctggatacgctgtgggttgatgaaaatgatgaaacatcgctgcgtaataatgttgcaaatagcacggcgaaagatttcggtgtgggaatggcttcaggtaccgatgcctataatggggcattaactaaagccacggcaaccaaccacgagaatggtgtgattactgtctacaacgccggtgcggggatggcggcgtatgggaatactaatacggtcattaaccagggaaccatcaacctggagaaaaatgaaaattacaacgattctctcggtgtgaataaactcgtcggcatggcggtttatcacgaaggcaccgccattaatgaccagacaggcgtaatcaacattaacgcggaaaatggtcaggcattctataacgatggcactgggcttattatcaactatggcacaatttgtacctttggcgtttgctag

SEQ ID NO: 5 - DNA, 693 bp, Escherichia coli
atgatgactaaaataaagttattgatgctcattatattttatttaatcatttcggccagcgtccatgctgccggagggatcgcattaggtgccacgcgtattatttatcccgctgatgctaaacagactgcggtatggattagaaatagccataccaatgagcgttttctggtcaattcgtggattgaaaacagcagcggtgtaaaagaaaagtcatttatcattacaccgccactatttgttagtgaacccaaaagcgaaaatactttgcgtattatttacaccggtccaccgctggcagcagatcgtgagtctctgttctggatgaatgttaagacgattccttcggtagataaaaatgcattgaacggcaggaatgttttgcaactggcgattttatcgcgcatgaagttatttctccgtccaattcaactacaagaattacccgcagaagcgccggatacactcaagttttcgcgatccggtaactatatcaatgttcataatccatcacctttttatgtcaccctggttaacttacaagtgggcagccaaaagttggggaatgctatggctgcacccagagttaattcacaaattcccttaccctcaggagtgcagggaaagctgaaatttcagaccgttaatgattatggttcagtaaccccggtcagagaagtgaacttaaactaa

SEQ ID NO: 6 - DNA, 462 bp, Escherichia coli
atgcagttcacttttaatgaaggtcatattcaactgccatcgcaatggcaggatcagtcgatgcaggtcctggtatccacggataacagtggcatcaacctggtcattacccgcgaacccgtttcacagggaaccctgacaccagagctgtaccaggaaacgctggcgctgtaccagggcaaacttgatgggtacaccgaacatgcctgccgggaaatcactctggcagaggccccggcctggctgctggattattcctggaacggtccggaagatgaagggaatcaggggcggattagccagatagccgtattccagcggcgtggagatacgctgctgaccttcactttcagcacctccttatccctgaagaacagtcaaaagacgatgctgctggaggtgataaagagcttcaccccgttgccaccggagaatgacatccaaaaggaccagccgcgctga

SEQ ID NO: 7 - DNA, 612 bp, Escherichia coli
atgaactcacaacaagggggcggtatgagccatgtctggggactgttttcccatcccgatcgtgaaatgcaggtgattaatcgcgaaaacgaaacgatttctcaccattacacccaccacgttttgctgatggcggcgattccggtgatttgcgccttcattggcactacacagattggctggaatttttgcgatggcactatcctgaagttatcctggtttaccggactggcgctggctgtcctgttttatggcgttatgctcgctggggtcgcggtgatggggcgggtcatctggtggatggcgcgtaattatccgcagcgtccgtcacttgcgcactgcatggtcttcgcgggctacgttgcaactccgctgtttttaagtggtctggtggcgctttatccgctggtctggttgtgtgcgctggtcggcacggtggcactgttttacaccggttacctactttaccttggtattccgagcttcctgaatatcaacaaagaggaaggccttagcttctccagttcgacgctggcgattggcgtgctggtactggaagtgctgctcgcccttaccgttatactatggggctacggttatcgtttgttctag

SEQ ID NO: 8 - DNA, 1194 bp, Escherichia coli
atgacgaaagtgacacgcgcagtaattgtgggaggcgggatcggcggtgcggcaactgcgctgtcactggcccgcctggggatcaaagtggtgctgctggaaaaggcgcatgagatcggtgagattggcgcgggcatccagttggggccaaatgcgttttcggcgctggacagcctcggcgtcggcgaagttgcccgccagcgcgcggtgtttaccgatcacattaccatgatggatgcggtgaacgccgaagaagtggtgtgcattgaaaccgggcaggcctttcgtgaccatttcggcggcccttacgcggtgatccaccgggttgatattcacgcctcggtgtgggaagcggtactgacgcatccggacgtggaatatcacacctccaccaacgtcgtggatattcgcgagacggaagacgatgtcaccgtgtttgacgatcgcggcaatagctggaccggcgatattctgattggctgtgacggcgtgaagtcagtggtgcgccagagcttattgggcgatgcgccgcgtgtcaccggacatgttgtctaccgtgcggttatcgatcgtgccgatatgccggaagatttgcgcattaacgcaccggtactgtgggcggggccgcattgtcacttggtgcattaccctttgcgcggcggcaagcagtataacctggtggtaacattccacagccgccagcaggaagagtggggcgtgaaggatggtagtaaagaggaagtgctctcttactttgaagggattcacccccgtccgcgccagatgttggataaaccgacttcatggcgacgctggtcaaccgctgaccgtgaaccggtggcgaagtggggaacgaagcgcataacgcttgtgggcgattcggcccatccggtggcgcaatatatggcgcagggagcctgtatggcgctggaagatgcagtaacgctgggtaaggcgctggcgcagtgtgagggtgatgcggcgcaagcctttgcgctgtatgagtcggtacgaattccgcgcaccgcgcgcatcgtctggtcgacccgtgaaatggggcgggtttaccacgccgcaggggtagaacgccaggtacgtaatctgctgtggaaagggaaatcgcaggcagaattttatcgcggcatggagtggttgtacggctggaaagaagataactgtttgcaaccacgctaa

SEQ ID NO: 9 - DNA, 1158 bp, Escherichia coli
atggagcgcaacgtcacgctcgactttgttcgcggcgttgccattctggggatcctgctattaaacatcagcgcctttgggctaccaaaggcggcttatcttaatcccgcctggtacggcgctattacgccgcaggatgcatggacctgggcatttctcgatctcatcggccaggtgaaattcctcacgctttttgcgctgctgtttggtgctggcctgcaaatgttgctgccccgtggcagacgctggatccagtcgcggttaacgctgttagtcttgctgggctttattcacggtttattgttctgggacggcgatattctgctggcttacgggctggtgggcttaatctgctggcggctggtgcgcgatgcgccatcggtaaaaagcctttttaataccggcgtcatgctttatctggtggggcttggcgttttgctgttattggggttgatttccgatagccagactagccgcgcctggacgccggatgcatcggctattttatatgaaaaatactggaagcttcacggcggcgttgaagcgatcagtaatcgtgccgatggtgttgggaacagtttattggcactgggcgcacagtatggctggcaactggctgggatgatgctcattggtgccgcattgatgcgcagtggctggctgaaagggcagttcagcttacgtcactatcgtcgtactggttttgtgctagtggcgattggggtgatcattaaccttcctgccatcgccctgcaatggcagctggactgggcgtatcgctggtgtgccttcttacttcaaatgccgcgggaactgagtgcgccgtttcaggcgattggctatgcgtcgctgttttatggcttctggccgcaattgagccgctttaagctggtgctggcgatcgcctgcgtcggacggatggcgctgaccaactatctattgcaaacgctgatttgtaccacgcttttttaccacctcggtctgtttatgcagtttgaccgtctggaattgctggcgtttgttattccggtatggctggcgaatattctcttctctgttatctggctgcgttacttccgccaggggccggtggaatggctctggcgtcagttaactttgcgtgctgccggaccggcaatatctaaaacatcaagataa

SEQ ID NO: 10 - DNA, 3882 bp, Escherichia coli
atgagccagcatttacctttggtcgccgcacagcccggcatctggatggcagaaaaactgtcagaattaccctccgcctggagcgtggcgcattacgttgagttaaccggagaggttgatgcgccattactggcccgcgcggtggttgccggattagcgcaagcagatacgctgcggatgcgttttacggaagataacggcgaagtctggcaatgggtcgatgatgcgctgatattcgaactgccagaaattatcgacctgcgaaccaatattgatccgcacggtactgcgcaggcattaatgcaggcggatttgcaacaagatttgcgcgtcgatagcggtaaaccactggtctttcaccagctgatacaggtggcagataaccgctggtactggtatcagcgttatcaccatttgctggtcgatggcttcagtttcccggccattactcggcagatcgccaacatttactgcgcattgctgcgtggcgaacaaacgcctgcttcgccgtttacgcctttcgctgatgtagtggaagagtaccagcaataccgcgaaagcgaagcctggcagcgtgatgcggcattctgggcggaacagcgtcgtcaactgccgccgcccgcgtcactttctccggcacctttagcggggcgcagcgcttcggcagatattctacgcctgaaactggaatttaccgacggggaattccgccagctggctacgcaactttcaggtgtgcagcgtaccgatttagcccttgcgctggcagccttttggctggggcgattgtgcaatcgcatggactacgccgccggatttatctttatgcgtcgactgggctcggcggcgctgacggctaccggacccgtgctcaacgttttaccgttgggtattcacattgcggcacaagaaacgctgccggaactggcaacccgactggcagcacaactgaaaaaaatgcgtcgtcatcaacgttacgatgccgaacaaattgtccgtgacagcgggcgagcggcaggtgatgaaccgctgtttggtccggtactcaatatcaaggtatttgattaccaactggatattcctggtgttcaggcgcaaacccataccctggcaaccggtccggttaatgaccttgaactggccctgttcccggatgaacacggtgatttgagtattgagatcctcgccaataaacagcattacgatgagccaacgttaatccagcatgctgaacgcctgaaaatgctgatcgcccaattcgctgcggatccggctctgttgtgcggcgatgttgatattatgctgccaggtgagtatgcgcagctggcgcagatcaacgccactcaggttgagattccagaaaccacgcttagcgcgctggtggcagaacaagcggcaaaaacaccggatgctccggcgctggcagatgcgcgttaccagttcagctatcgggaaatgcgcgagcaggtggtggcgctggcgaatctgctgcgtgagcacggcgttaaaccaggggacagcgtggcggtggcattaccgcgctcggtctttttgaccctggcgctacatgcgattgttgaagcaggtgcggcctggttaccgctggataccggttatccggacgatcgcctgaaaatgatgctggaagatgcgcgtccgtcgctgttaatcaccaccgacgatcaactgccgcgctttgccgatgttccagatttaaccaacctttgctataacgccccgcttacaccgcagggcagtgcgccgctgcaactttcacaaccgcatcacacggcttatatcatctttacctctggctccaccggcaggccgaaaggggtaatggtcgggcagacggctatcgttaaccgcctgttgtggatgcaaaatcattatccacttacaggtgaagatgtcgttgcccaaaaaacgccgtgcagttttgatgtctcggtgtgggagtttttctggccgtttattgccggggctaaactggtgatggctgaaccggaagcgcaccgcgacccgctcgctatgcagcaattctttgccgaatatggcgtaacgaccacgcactttgtgccgtcgatgctggcggcatttgttgcatcgctgacgccgcaaaccgctcgccagaattgcgcgacgttgaaacaggttttctgtagtggtgaggccttaccggctgatttatgccgcgaatggcaacagttaacgggcgcgccgttgcataatctatatggcccgacggaagcggcggtagatgtgagttggtatccggcttttggcgaggaactggcacaggtgcgcggcagcagtgtgccgattggttatccggtgtggaatacgggcttgcgcattctcgatgcgatgatgcatccggtgccgccgggtgtggcgggagatctctatctcaccggtattcaactggcgcaggggtatcttggacgacccgatctgaccgccagccgcttcattgccgatccttttgtccctggtgaacggatgtaccgtaccggagacgttgcccgctggctggataacggcgcggtggagtacctcgggcgcagtgacgatcagctaaaaattcgcgggcagcgtatcgaactgggcgaaatcgatcgcgtgatgcaggcgctgccggatgtcgaacaagccgttacccacgcctgtgtgattaaccaggcggcagccaccggtggtgatgcgcgtcagttggtgggctatctggtgtcgcaatcaggtctgccgttggataccagcgcattacaggcacagcttcgcgaaacattgccgccgcatatggcgccggtcgttctgctgcaacttccacagttacctcttagcgccaacggcaagctggatcgcaaagccttaccgttgcctgaacttaaggcacaaacgccggggcgtgcgccgaaagcgggcagtgaaacgattatcgctgcggcattcgcgtcgttgctgggttgtgacgtgcaggatgccgatgctgatttcttcgcgcttggcggtcattcgctactggcaatgaaactggcagcgcagttaagtcggcagtttgcccgtcaggtgacgccggggcaggtgatggtcgcgtcaaccgtcgccaaactggcaacgattattgatggtgaagaggacagctcccggcgcatgggattcgaaaccattctgccgttgcgtgaaggtaatggcccgacgctgttttgtttccatccggcatccggttttgcctggcagtttagcgtgctctcgcgttatctcgatccactatggtcgattatcggcattcagtcgccgcgccctcatggccccatgcagacagcgacgaacctggatgaagtctgcgaagcgcatctggcaacgttacttgaacaacaaccgcacggcccttattacctgctggggtattcccttggcggtacactggcgcagggcattgcggcgcgactacgtgcccgtggcgaacaggtggcatttcttggcttgctggatacctggccgccagaaacgcaaaactggcaggaaaaagaagctaatggtctggacccggaagtgctggcggagattaaccgcgagcgcgaggccttcctggcggcacagcagggaagtacttcaacggagttgtttaccaccattgaaggcaactacgctgatgctgtgcgcctgctgacgactgctcatagcgtaccgtttgacggaaaagcgacgctgtttgttgctgaacgtacgcttcaggaaggtatgagccccgaacgcgcctggtcgccgtggattgcggagctggatatctatcgtcaggattgtgcgcatgtggatattatctctccaggggcatttgaaaaaattgggccgattattcgcgcaacgctaaacagataa

SEQ ID NO: 11 - DNA, 1533 bp, Escherichia coli
atgacgcagaaaattgaacaatctcaacgacaagaacgggtagcggcctggaatcgtcgcgctgaatgcgatcttgccgctttccagaactcaccaaagcaaacctaccaggctgaaaaagcgctcgatcgcaaactgtgcgccaacctggaagaagcgattcgtcgctctggtttacaggacggcatgacggtttccttccatcacgctttccgtggcggtgacctgaccgtcaatatggtgatggacgtcatcgcgaagatgggctttaaaaacctgatcctggcgtccagctccctgagtgattgccatgcgccgctggtagaacacattcgccagggcgtggttacccgcatttatacctccggcctgcgtggtccactggcggaagagatctcccgtggtctgctggcggaaccggtgcaaatccactctcacggcggacgtgtgcatctggtacagagcggcgaactgaatatcgacgtggctttcctcggcgtcccgtcctgtgatgaattcggtaatgccaacggctacaccggtaaagcctgctgcggctccctcggctatgcaatggttgatgccgacaacgcaaaacaggttgtgatgcttaccgaagaactgctgccttatccgcataatccggcaagcattgagcaagatcaggttgatttaatcgtcaaagttgatcgcgttggcgatgctgcaaaaatcggcgctggtgctacccgtatgaccactaacccgcgcgaactgcttattgcccgtagcgctgcggatgtgattgtcaactcaggctacttcaaagaaggtttctccatgcaaaccggcaccggcggcgcatcgctggcggtaacccttttcctggaagacaaaatgcgtagccgcgatattcgcgccgacttcgccctcggtggtattaccgcgacgatggttgatctgcacgaaaaaggtctgatccgcaaactgctggatgtacagagctttgacagccacgctgcgcaatcgctggcccgtaaccccaatcacatcgaaatcagcgccaaccagtacgctaactggggttcgaaaggtgcatcggttgatcgtctcgacgtggtggtactgagcgcgctggaaattgacacccagttcaacgttaacgtgctgaccggctctgacggcgtactgcgtggtgcttccggtggtcactgcgataccgcgattgcctctgcgctttccatcatcgtcgcgccgctggtacgcggtcgtattccgactctggtggataacgtgctgacctgcatcaccccgggctccagtgtcgatattctggtcacagaccacggtatcgcagttaacccggcacgtccggaactggcagaacgtctgcaggaagcgggcattaaagtggtttccattgagtggctgcgcgaacgtgcgcgtctgctgaccggtgaaccacagccgattgaattcaccgaccgcgtcgttgccgttgtgcgttaccgcgatggctcggtgatcgatgttgtgcatcaggtgaaggaataa

SEQ ID NO: 12 - DNA, 1647 bp, Escherichia coli
atgaaaaaagtgactgccatgctcttctcgatggccgtggggcttaatgccgtttcgatggcggcaaaagcgaaagcgtccgaggagcaggaaactgatgtactgttgattggcggcggcattatgagcgccacgttggggacctatttacgcgagctggagcctgaatggtcgatgaccatggtggagcgcctggagggtgtcgcgcaggagagttcgaacggctggaataacgccggaaccgggcattctgcactgatggaactgaactacaccccgcaaaacgccgatggcagcatcagtattgaaaaagcagtcgccattaacgaagcatttcagatttcccgccagttctgggcgcaccaggtcgagcgcggcgtgctgcgtactccgcgttcatttatcaataccgttccgcatatgagctttgtctggggcgaggataacgtcaatttcctgcgcgcccgttacgccgcgttgcaacaaagctcgctgtttcgcggtatgcgttactctgaagatcacgcgcagatcaaagagtgggcaccgttagtgatggaagggcgcgatccgcaacagaaagtggcagccacgcgtacggaaattggtaccgatgtgaactacggcgagatcacccgccagttaattgcttccttgcagaagaaatctaacttctcgctgcaactcagcagcgaagtccgcgccctaaagcgtaatgacgataacacctggaccgttaccgttgccgatctgaaaaatggcactgcacagaacatccgtgcgaaatttgtctttatcggcgcgggcggtgcggcactgaagctgttacaggaatcggggattccggaagcgaaagactacgccggtttcccggtgggcggacagttccttgtttcggaaaacccggacgtggttaatcaccatctggcgaaggtttacggtaaagcatccgttggcgcaccaccgatgtcggttccgcatatcgatacccgcgttctggacggtaaacgcgtagtgctgtttgggccatttgccaccttctcaaccaaattcctcaaaaacggttcattgtgggatctgatgagttccaccaccacctctaacgtgatgccgatgatgcacgtcgggctggataatttcgatctggtgaaatatctggtgagtcaggtgatgttgagtgaagaggatcgttttgaagcgttgaaagagtactatccgcaagcgaaaaaagaggactggcgtttgtggcaagcggggcagcgtgtgcagattatcaagcgtgatgccgataaaggtggtgtactgcgtctgggtactgaagtggtcagtgaccagcaaggaaccattgccgcgctcctgggggcatcgccaggggcgtcaaccgccgcgccgattatgttgaatctgctggaaaaagtatttggcgatcgcgtttccagcccgcaatggcaggctacgttgaaagcgatcgttccatcttatggacgcaagctgaacggtgatgtagcggcaacagaacgcgagttgcagtacaccagcgaagtgctggggctgaaatacgacaaaccgcaagcagcagatagtacgccgaaaccgcagttgaaaccgcaacccgttcaaaaagaagtggcggatattgcgttgtaa

SEQ ID NO: 13 - DNA, 2640 bp, Escherichia coli
atgcctgaccattctcttttccgactgcgggtacttccctgctgcgttgcattggcaatgtccgggagttatgtcaatgcctgggctgaaaatgaaattcagtttgattcccgttttctggagttaaaaggcgacacaaaaatcgatctgaagcgattttccagccagggttatgtcgaacctgggaaatacaatttacaggttcaactaaataaacagccgctgacggaagaatacgatatttactggtacgcctctgagaacgatgccagtaaaacctatgcctgcctgacgcctgaactggtcgcgcagtttggcttaaaagaggatgtggcaaaaaacctgcaatggatccacgacggcaaatgcctgaaacccggtcaactggaaggcattgatattaaagctgacctgagtcagtcagcgttagtcatttcattaccccaggcttaccttgaatataccgacatcaactgggatccgccttcacgctgggatgacggtatatctggtttaattgctgactacagtattaccgcccagacacgacatgaagaaaatggcggggatgacagcaatgaaattagcggtaacgggacggttggggtgaacctcggcgcatggcgtcttcgtgccgactggcagactgattatttgcatagtaaaagcaatgatgacgatgttatcaacggtgatgacacgcaaaaaaactgggagtggagccgctactacgcctggcgagccttaccgtcgctaaaagccaaacttggccttggcgaagactacctgaattctgatattttcgacggctttaactacgtgggtggcagtatcagcaccgacgatcaaatgttgccgccgaatctgcgcggctatgcgccggatatctccggcgtggcgcacaccaccgcgaaagtgaccgtcagccagttgggccgcgtcatctacgaaacccaggtcccggcggggccgttccgcatccaggatcttggcgattcggtctccggtacgctgcatatccgcattgaagaacagaacggtcaggtgcaggaatatgacatcaacaccgcctcgatgccgttcctgactcgccccggccaggtgcgctataaactgatgatgggccgcccgcaggagtgggggcaccacgtggaaggcggtttcttctccggcggcgaagcttcctgggggattgccaacggctggtcgctatacggcggggcgctggcagatgaacactatcagtcggcggcgcttggcgtcggtcgcgacctgtctgtgtttggtgcggtggcctttgatatcacccactcgcatacccgtctggataaagagaccgcctacgggaaaggttcactggacggcaactcgtttcgcctgagctattccaaagacttcgatgaactgaacagccgcgtcacttttgccggataccgcttctcggaagagaacttcatgaccatgagcgagtatctcgatgccagcgacagcgaaatggtgcgcaccggcaacgacaaagagatgtacaccgccacctataaccagaacttcagggatgccggtgtgtctgtttatctcaactacacccgccatacctactgggatcgcgacgaacagaccaactacaacgtcatgctctcgcactacttcaacctgggcagtatccgcaacatgagcatttccatgaccggataccgctacgagtatgacaaccaggccgataaaggtgtgtacatatcgctcagtatgccgtggggtgacagcagcaccatcagctataacggcaactacggcagcggttcggacagcagccaggtggggtatttcagccgtgtcgatgacgcaacccattaccagttgaacgtaggcaccagcgacaatcactccagcgttgacggttattacagccacgacggatcgctggcgcaggtcgatctcagcgctaactaccatgaagggcagtacacctcggcgggtatttccttacagggcggcgcgacgctcaccgcacaaggtggcgcgctccaccgtacccagaatatgggcggtacgcgtctgctgattgatgccgacggtgtggctggtgttccggtggaaggaaatggcgcggcggtttacaccaatatgttcggtaaggcagtggtggcagacgtcaacaactactaccgcaaccaggcgtatatcgacctaaacaacctgccggaaaacgccgaagccacccagtccgtggtgcagggcacgcttaccgaaggggccattggctaccgtaagttctcggtgatcagcgggcaaaaagcgatggcggtgctgcgtctgcaagatggcagttatccgccgtttggcgcggaagtgaaaaacgacagcgcgcagaacgtcggtctggttgacgatgacggcaacgtctacctcgcgggcgtaaaacctggcgagcatatgatcgtttcatggggcggtgtggcccactgcgatattcatctgcctgacccgctgccagccgatctgttcaatggcctgttattaccatgccagcaaacaggggcgatatctccttcgatgcctcatgaaattaagccggtgatccaggagcagacccagcaggtgatgccaacggaagcgccagtatcggtatcagccaattaa

SEQ ID NO: 14 - DNA, 924 bp, Escherichia coli
atgggaggattgccaatgatttccgcttcgctgcaacaacgtaaaactcgcacccgccgcagcatgttgtttgtgcctggtgccaatgccgcgatggtcagcaactccttcatctacccggctgatgccctgatgtttgacctcgaagagtccgtggcattacgtgaaaaagacaccgcccgccgcatggtttatcacgcgctgcaacatccgctgtatcgcgatattgaaaccattgtgcgtgtaaacgcgctggattccgaatggggtgttaacgacctggaagccgtcgttcgcggtggtgcggacgttgtacgtctgccgaaaaccgataccgctcaggatgttctggatatcgaaaaagagatcctgcgtatagaaaaagcctgtggtcgtgaacccggcagcaccggcctgctggcggcgattgaatctccgctgggcattacccgcgcagtggaaatcgctcacgcttccgagcgtttgatcggtatcgccctcggtgcagaagactatgtgcgcaacctgcgtacagaacgctccccggaaggaactgaactgctgttcgcacgctgttccattttgcaggccgcgcgctctgcgggtattcaggcgttcgataccgtctattccgacgctaacaacgaagccggattcctgcaagaagccgcccacatcaaacagctgggctttgacggcaaatcgctgatcaacccgcgtcagattgatctgctgcacaacctctacgcaccgacccagaaagaagtggatcacgcccgccgcgtcgtagaagccgctgaagccgccgctcgcgaaggcctcggcgtggtttccctgaacggcaagatggtggacggtccggttatcgatcgtgcccgtctggtgctctcccgtgcagaactttccggcatccgcgaagaataa

SEQ ID NO: 15 - DNA, 909 bp, Escherichia coli
atgccgggtacttatcagggtgcggaggccggggcgaattttgattacggcgatgctggtgcgttgagtttctcctacatgtggaccaacgaatacaaagcaccgtggcatctggaaatggatgagttttatcagaacgataaaaccaccaaagttgattatctgcactcccttggggcgaaatacgacttcaaaaataacttcgtactggaagcggcatttggtcaggcggaagggtatatcgatcaatactttgccaaagccagctacaaatttgatatcgccggtagcccgttaaccaccagctaccagttctacggtacgcgcgataaagttgatgatcgcagcgtcaacgatctttatgacggcaccgcctggctgcaagcgttgacctttggttaccgggcggctgacgtagtggatttgcgcctcgaaggcacctgggttaaagctgacggtcagcagggatacttcctgcaacgtatgactccaacctacgcttcctcgaacggtcgcctggatatctggtgggataaccgttctgacttcaacgccaacggcgaaaaagcggtcttcttcggtgcgatgtatgacctgaaaaactggaatcttccaggcttcgccatcggcgcttcctacgtttacgcatgggatgctaaacctgcgacctggcagagcaatccggatgcgtactacgacaaaaaccggactattgaagagtctgcatacagcctggatgcggtctacaccattcaggacggtcgcgccaaaggcacgatgttcaaactgcacttcaccgaatacgacaaccactccgacatcccaagctggggcggcggttacggcaacatcttccaggatgagcgtgacgtaaaatttatggtaatcgcaccattcaccatcttctga

SEQ ID NO: 16 - DNA, 2199 bp, Escherichia coli
atgtcagaattaaaaattgccgttagtcgttcttgcccggattgtttttccactcatcgtgcatgcgtgaatatagacgaaagtaattatattgacgttgccgccattattttatcagtcagtgatgttgaacgtggaaaactcgatgaaatagacgctactggctatgacattcctgtttttattgcaacggaaaatgaagaacgtataccagcagaatatctttcacgtatttctggtgtcttcgaacatggagaagcacgcaaagaattttatggtcgtcagttagaaaccgctgccagccattatgaaactcaactgcgcccacctttctttcgcgcactggtcgattatgtcaatcagggcaacagcgcatttgactgccccggtcatcagggcggcgaatttttccgtcgccatccggcggggaatcagtttgtggaatactttggtgaggcgctgttccgtgccgacttgtgcaacgccgacgtagcgatgggcgatctgctgattcacgaaggcgcgccatgcattgcacagcaacatgcagcaaaagtgtttaatgccgataaaacctacttcgttttaaatggcacttcatcttctaacaaagtggttttaaacgccctgctgacaccgggtgatctggtgctatttgaccgcaataaccataagtctaaccaccacggggccttgctacaggctggtgcaacaccggtttatctggaaacagcacgtaacccgtatggttttatcggtggtattgatgcacactgcttcgaagaaagttatctgcgtgaactgattacagaagttgcaccgcaacgggcaaaagaggcgcgtccgttccgcctcgctgtgattcagctaggcacctacgacggtacgatttataacgcccgccaggtggtggataaaattggtcatctgtgtgactacatcctgtttgattccgcatgggtcggctatgaacagtttattccgatgatggctgactgttcgccgctgttgctggatcttaatgagaacgatccgggtattctggttacgcaatccgtacataaacagcaggctggtttttctcaaacttcacaaatccataaaaaagacagccacatcaaagggcaacagcgttatgtaccgcacaaacgcatgaacaacgcctttatgatgcatgcctccaccagcccgttctatccgctgtttgccgcactggatatcaacgccaaaatgcatgaaggtgtcagcggtcgtaatatgtggatggattgtgtggtaaatggcattaatgcccgcaaactgatcctcgataactgtcagcatattcgtccgttcatacctgaactggtggatggtaaaccctggcagtcgtatgaaacagcgcaaattgcggttgatctgcgcttcttccagtttgtaccaggtgagcactggcattcttttgaaggctatgcagagaatcaatactttgttgatccgtgcaaactgttgctgacaaccccaggtattgatgcacgtaacggcgaatatgaagcgttcggtgtacccgcgacgattcttgctaacttcctgcgcgaaaatggcgtagtgccggaaaaatgcgatcttaactccatcctcttcctgctgactccggcagaagatatggccaaacttcagcaacttgttgccctgctggtacgcttcgaaaaactgctggaatctgatgcgccgctggcggaagtgctaccttccatctacaaacagcatgaagagcgctacgccggttatacccttcgtcagttgtgccaggaaatgcatgatttgtatgcccgccacaacgtgaaacaactgcaaaaagagatgttccgtaaggaacacttcccacgcgtcagcatgaatccgcaagaagctaactacgcctatttacgcggtgaagtggagctggttcgtctgccggatgcagaaggccgtatcgctgccgaaggtgcgcttccttatcctccgggtgtgctgtgtgttgttccgggtgaaatctggggtggtgctgttttgcgttacttcagcgctctggaagaagggatcaacctgctgccaggttttgcaccggagctgcagggtgtctatatcgaagaacatgatggtcgtaagcaagtttggtgctatgtcatcaagcctcgtgatgcgcaaagcaccctgctgaaaggggaaaaattatga

SEQ ID NO: 17 - DNA, 1572 bp, Escherichia coli
atgatgaaaaccatcaccaaacaaccgatcctgtttactgatgtaccggttgcagatttaagaaactcaatgaagcaagatttaaatcaaaatcttattgaaaggctgtggaacaaaattcgtgatttttttctggacagtgataaacaaaaggcatttaaatcgattcataaatacatcaatacattgtcagttctaaattataattcagcgttaactccagatcccaactttaatatagatgcaacgtcagatttagactcatatttaaagctggattttgatcgcctctcgcccaaacaaaaacagaccacgctttgttgtttctggaataaaattgcatcctcattgccagagccatacaacagtaccatcaaacataacattattttttataaagacggagaaaatctgatgatacgtggaactatctctattgttaacgaggtagtaaagacttattcactcccaatagaaaaggatgacaatggatattacgatttcagcggtctatatctagcacacagcaatatttccggtaaagatcccaataaagatcccgatatagatttcggtatagatatgggaaactgtaactgctcaaatgtcaacttcgaacatacttatttttatggcgtcaagtttacaaatgcgaattgtacaaatgcgaattttaacaattgtagatttaagaaatgcgacctgacaaatatgaattgcactggcgcaattcttgataatgcgatgatatatggtaaagaaaaagaacctgagatgcaatatccagaagcagatcaaataatacaaaggataacttatcagaaatcagatgggaatgaaacgaaagggatgattctaactaactgctcctgtgtgaaaaccactttcaattgggcagatttatcagaaagtgattgccaaaatgtggactttagcgaggctaatctttcaaacactattcttcctgacatcgtcaggatgaaaggcacgaaactttaccgtaccgacctttttaatccgatattaaaaacagaagcagaatcaacagaagaaaaagacattagcccattagcaaaaattattcttgactatattgaatcagataaaaaccctgagtcattaaactttgaagaaaaatcaacagttattaaaataaaacaagatatcgataattttattttctacaatcagcatttaaagaaaatattcaatcgcgcaatgaatcttcaagaaaaaataagcaggaaaaagtataatgaattttttaaatatatacaagcagaagccaaacaatatttcaaagaccagtataaactaacaaaaaatgattatttgaagaaagtacctttaactgcacaattaattgcaaaatacaaaatggatgatcaattggaccaacttttggttacccgtgaaattcaagatgaaataaaatcaaagattcaagacaaaatagatgaactttctaaaaatttattcaacactatgaccgaaaccattgagaataattttgatgatatttttcgtcaacagtcagagaatatgagcaattattatgagttcgtcgactga

SEQ ID NO: 18 - DNA, 924 bp, Escherichia coli
gtgacgtttgtaccactgagtccgatcccgttaaaagatcgcacctctatgatcttcctccagtacggtcaaatcgacgtactggacggcgctttcgtgctgatcgacaaaaccgggatccgcacgcacattccggtgggatcggtcgcctgcattatgctcgaaccgggaacgagagtttcccacgcggcggtgcatctggccgccacggtgggaacactgctggtctgggtcggtgaagcgggcgttcgcgtttactcttccggacaacccggaggggcgcgggcagataaattactctaccaggcaaagctggctttaacggaagatctacgcctgaaggtggtgcgcaaaatgtatgaattacgttttcgtgagccaccgccagctcgccgttcagtggatcagctacggggaattgagggatcccgcgttcgccagacctatgcattactggcgaaacaatatggtgtgaaatggaatggtcgcaaatacgatcctaaagactgggaaaaaggcgatgttgtgaatcgctgcatcagtgctgccacatcatgtctgtacggtatttctgaagcggcagtattagccgcgggatatgcgcccgctattggatttattcatagtggcaaaccgctttcatttgtttatgacatagccgatatcattaaatttgattcggttgtgccaaaggcatttgaaatagcagcgaggcaacccgcagaacctgataaagaagtcagattagcctgtcgcgatattttccgtagcactaagttaacgggcaaattaataccgttaattgagaaagtccttgctgcaggtgaaattgaaccaccacaacccgcgccggatatgttaccgcctgccatccctgaacctgaaacgctgggtgatagtggtcaccgggggcgcggcggatga

SEQ ID NO: 19 - DNA, 996 bp, Escherichia coli
atgattaatgaagccacgctggcagaaagtattcgccgcttacgtcagggtgagcatgccacactcgcccaggccatgacgctggtggaaagccgtcacccgcgtcatcaggcactaagtacgcagctgcttgatgccattatgccgtactgcggtaacaccctgcgactgggcattaccggcacccccggcgcggggaaaagtacctttcttgaggcctttggcatgttgttgattcgagagggattaaaggtcgcggttattgcggtcgatcccagcagcccggtcactggcggtagcattctcggggataaaacccgcatgaatgacctggcgcgtgccgaagcggcgtttattcgcccggtaccatcctccggtcatctgggcggtgccagtcagcgagcgcgggaattaatgctgttatgcgaagcagcgggttatgacgtagtgattgtcgaaacggttggcgtcgggcagtcggaaacagaagtcgcccgcatggtggactgttttatctcgttgcaaattgccggtggcggcgatgatctgcagggcattaaaaaagggctgatggaagtggctgatctgatcgttatcaacaaagacgatggcgataaccataccaatgtcgccattgcccggcatatgtacgagagtgccctgcatattctgcgacgtaaatacgacgaatggcagccacgggttctgacttgtagcgcactggaaaaacgtggaatcgatgagatctggcacgccatcatcgacttcaaaaccgcgctaactgccagtggtcgtttacaacaagtgcggcaacaacaatcggtggaatggctgcgtaagcagaccgaagaagaagtactgaatcacctgttcgcgaatgaagatttcgatcgctattaccgccagacgcttttagcggtcaaaaacaatacgctctcaccgcgcaccggcctgcggcagctcagtgaatttatccagacgcaatattttgattaa

SEQ ID NO: 20 - DNA, 1326 bp, Escherichia coli
atgagcaaagtaactccccagccgaaaatcggctttgtttcccttggctgtccgaaaaaccttgtcgattcagagcgtattctcaccgaactccgcactgaaggttatgacgtggtaccgagctatgacgatgcggacatggtgatcgtcaacacctgcggctttattgacagcgcggtacaagaatcactggaagccattggtgaagcgttgaatgaaaacggcaaggtaattgtgaccggttgtctgggggcaaaagaagatcagatccgcgaagtccacccgaaagtgctggaaatcaccgggcctcatagctatgagcaggttctggagcacgttcatcactacgtgccaaaaccgaaacacaacccattcctgagcctggtgccagaacaaggtgtcaaactgacgccgcgtcattatgcctatctgaaaatttctgaaggctgtaatcaccgctgcaccttctgcattattccgtctatgcgcggcgacctggtgagccgtccgattggcgaagtattaagtgaagcgaaacgtctggtagatgcgggcgttaaagagattctggtgatctcgcaggatacttccgcctatggcgttgatgttaaacatcgtactggcttccacaacggcgagccggtaaaaaccagcatggtcagcctgtgcgaacagttatcgaaactggggatctggacacgtctgcactacgtttacccttatccgcatgtggacgacgtcatcccactgatggcagaaggcaaaatcctgccgtatctggacattccgttgcagcacgccagcccgcgcattctcaaactgatgaagcgtccgggttctgtagatcgccaactggcgcgcatcaaacagtggcgcgaaatctgcccggaactgaccctacgctcaacctttattgtcggcttccctggcgagacggaagaagatttccagatgctactcgacttcctgaaagaagcgcgtctggatcgcgttggctgctttaaatacagcccggttgaaggtgcagacgccaatgccctgcctgaccaggttccggaagaagtgaaagaagaacgctggaaccgtttcatgcagttgcagcagcagatttccgccgagcgcctgcaagagaaagtgggccgtgaaattctggtgattatcgacgaagtggacgaagaaggcgcgattggtcgcagcatggcagatgcaccggaaatcgacggtgcggtttatctcaatggtgaaaccaacgttaagccgggtgatatcctgcgtgtgaaagtcgagcacgccgatgagtacgatttgtggggtagccgggtttaa

SEQ ID NO: 21 - DNA, 1482 bp, Escherichia coli
atgagagcgattattctgctgttcgatagtctgaataaacgttatctgccaccttatggcgatgcgttaaccaaagcgcctaatttccaacgtctggcggctcatgccgccacctttgaaaacagttacgtcggcagtatgccgtgtatgcctgcccggcgggagttgcataccgggcgatgtaacttcctgcatcgggagtgggggccgttagaaccgtttgacgattccatgccggagctgcttaaaaaggcggggatctatacccacctgataagcgatcatttgcattactgggaagatggcggcgagaattatcataatcgctacagttcgtgggagattgtgcgcggacaggaaggcgatcactggcatgcaagcgttgcgcaaccgcctattcccgaggtgctacgggtgccgcaaaagcagaccggtggcggtgtttctggtctgtggcgccatgactgggcaaaccgggaatatattcagcaggaagcggattttccacaaactaaagtgtttgatgcaggctgtgcctttatccacaaaaatcacgcggaagataactggttattgcagatcgaaacgtttgatccgcacgagccgtttcacacaacggaagagtatctttccttgtatgaagataactgggatggaccgcattatgactggccgcgtggccgggtgcaggaaagcgacgaggccgtggagcacatccgttgccggtatcgttcgctggtgtcgatgtgcgatcgcaacctggggcgcattctcgacctgatggatgagcacgatctgtggcgagataccatgctgattgtcggcactgaccacggctttttactcggtgagcatggctggtgggcgaaaaaccagatgccttattacaacgaagttgccaataatccgttgtttatctggaacccgcgcagtggtgtaaaaggagagcggcgacaggcactggtacaaatgatcgactgggcgccaacgttgtatgacttttttcaacagccagtgccgcccgatgtgcaggggcaaccgctggcgaaaacggtcagtcacgatgaaccagtacgcagctcggcgatgtttggtgttttcagtggtcatgctaacgtaactgatgggcgttatgtgtatatgcgtgcagcgctgccggggcgtgaggatgatattgccaactacacgttgatgtcctgcaaaatgaacagccgctatccggtggatgagatgcgggctttatcgctggccccaccgtttcgttttaccaaagggttacaggtattacgcatcccggcacaggaaaaatataaggggttgaatcagtttggtcatttgctgtttgatctgcaaaacgatccgcagcagctacatccgattcatgatgatgtgatcgagtcccggatgatcgcgttgctgattcagttgatgaaagataatgatgcgccgccagagcagtttcagcgcctgggattagcgtag

SEQ ID NO: 22 - DNA, 594 bp, Escherichia coli
atgcaaaaagttgtcctcgcaaccggcaatgccggtaaagtgcgtgagctggcgtcgctgcttagcgacttcggtcttgatatcgtggcccaaacagaactcggcgttgattccgctgaagaaaccggcctgacctttatcgaaaacgcgattctgaaagcgcgccacgcggcaaaagtgaccggtttaccggcaattgccgacgactctggcctggcggtagatgtgcttggcggcgcgccgggtatttactccgcacgttattccggtgaagacgcgaccgatcaaaagaatctgcaaaaactgctggaaacactgaaagacgtaccggacgaccaacgtcaggcgcgtttccactgcgtgctggtgtatctgcgtcacgcagaagatccgaccccgctggtgtgccatggcagctggccgggtgtgattactcgcgaaccggcgggcactggtggctttggttatgatccaatcttcttcgtaccttccgaagggaaaaccgctgccgaactgacccgcgaagaaaagagcgccatttcccaccgtggtcaggcattgaaactgctgctggacgctttacgtaatggttaa

SEQ ID NO: 23 - DNA, 987 bp, Escherichia coli
gtgaacagacgtaattttattaaagcagcctcctgcggggcattgctgacgggcgcgctgccgtctgtcagtcatgcggctgctgaaaaccgcccgccaattccgggatcgctggggatgttgtacgactcgaccttgtgcgtaggctgccaggcttgcgtcaccaagtgtcaggatatcaacttccctgaacgtaacccgcaaggggaacagacctggtcgaacaacgacaaactgtcgccgtataccaataacatcattcaagtgtggaccagcggcacaggggtcaacaaagaccaggaggagaacggctacgcgtacattaagaaacagtgtatgcactgcgtcgatccgaactgtgtctctgtgtgcccggtctctgcactgaaaaaagatccgaaaaccggcattgtccattacgacaaagacgtgtgcactggttgccgttactgcatggtcgcctgtccgtacaacgtgccgaagtacgactacaacaacccgtttggtgcgctgcataagtgcgagctgtgcaaccagaaaggtgtggaacgtctcgataaaggcggtctgcctggctgcgtagaagtgtgcccggcgggcgcggtgatttttggtacgcgtgaagagctgatggcggaggcgaaaaaacgtctggcgctgaagcctggcagcgaataccactatccgcgtcagacgctgaaatctggcgacacttacctgcatacggtgccgaaatattatccgcatctgtacggcgagaaagagggcggcggtactcaggttctggtactgacgggtgtgccttatgaaaatctcgacctgccgaaactggacgatctttctaccggtgcgcgttccgaaaatattcaacacaccctgtataaaggcatgatgctaccactggctgtgctggcgggcttgaccgtgctggttcgtcgcaacaccaaaaacgaccatcacgacggaggagacgatcatgagtcatga

SEQ ID NO: 24 - DNA, 2142 bp, Escherichia coli
atggctaagttcacaccttcattctcaggaatcaaaggtcgggcgctcttttcactgctctttgcagcaccaatgattcatgcaaccgacactgcaacgaccaaagatggcgaaacaatcactgttacagcggatgcaaataccgcaactgaggcgaccgatggttatcaacctctgagcacttccacggcgacattaaccgatatgccgatgctggatatcccgcaggtggtcaatacggttagcgatcaggttctggaaaatcagaatgcaacgacgctggatgaagcgctttataacgtcagtaacgtggtacagaccaatacattaggcggaactcaggacgcctttgtacgtcgtggttttggtgctaaccgggatggctccatcatgaccaacggcctgcgaactgtacttcctcgcagtttcaacgccgccacagaacgtgtggaagtgctaaaaggtccggcctccacgctgtatggcattctcgatcctggtggattgattaacgtcgtgaccaagcgcccggaaaaaacattccatggttctgtctcagccacgtcctccagttttggcggaggcactgggcaacttgatatcacaggtcccattgaaggcactcagctggcataccgcctgacgggggaagtgcaggatgaagattactggcgaaacttcggtaaagagcgcagtacatttattgccccgtcactcacctggtttggtgataatgcaacagtaaccatgctctattcccatcgggactataaaactccgttcgatcgtggaacgattttcgaccttacgacgaaacagcccgtaaacgttgatcgaaaaatacgttttgacgaaccgtttaatattacagatggtcagtccgatctggcgcaactcaacgcagaatatcatctcaatagccagtggacagcgcgctttgattacagctacagccaggataaatacagcgacaatcaggctcgcgttaccgcgtatgatgcaacgacaggaacgctgacacggcgtgttgatgcaactcagggatctacccagcgtatgcattctactcgtgcggatctgcaagggaatgttgatattgctgggttctataatgagattctgggtggggtgtcatatgaatattatgatcttctgcgcacagatatgattcgctgtaaaaacgctaaagatttcaatatctacaaccccgtttatggcaataccagcaaatgcacaacggtttcggcgtcggacagtgatcagacgatcaaacaggagagctactcagcttatgcacaggatgcgctctatctgaccgataactggattgccgtcgccgggatccgctatcagtattacacgcagtatgcgggtaaaggccgtccttttaatgtcaatactgacagccgcgatgaacaatggacgcccaaactggggttagtctacaaactgacgccatcggtatccttatttgctaattattcgcaaacatttatgccgcaatcgtcaattgccagctacatcggagatcttccaccggaatcatctaatgcttacgaagtcggggcaaaattcgagctgttcgatggtataaccgcagatattgcgctgtttgatatccataaacgtaacgtgttgtataccgaaagtattggtgatgaaaccatcgccaaaacggcaggccgcgttcgttcaagaggggtagaagtcgaccttgcgggagcattaactgaaaacattaatatcattgccagctacggctataccgatgctaaggttctggaagatcctgattatgcagggaaaccattgccgaatgttcctcgtcataccggttcgctattcctgacctatgacattcataacatgccaggcaataacacactgacgtttggcggtggtggacattgcgtaagccgtcgttcggcaaccaatggggctgactattatctgccaggctatttcgttgccgatgccttcgccgcatacaaaatgaaattgcagtatccggtcacactgcaattaaacgtcaaaaacctgtttgataaaacgtattacacctcttccatcgccacaaataatctggggaatcagattggcgatccgcgtgaagtgcaattcacggtgaaaatggaattttga

SEQ ID NO: 25 - DNA, 1533 bp, Escherichia coli
atgcgtggggaaacatgtatggtattcaacaaaaaaatgtttgtactcattataataccaggcattctgggggtgttactgtcgtttgctatgtcagtatttcaaatgaatcgagatacgacaattaccgccggtattctgttaaaacagcttgataatgttacgcaaattgtcaaacatacgacaaagcttacttctattctggtgatgaaaccctgcaaagatatcctggaacaattgattgctaacggagcattgaccccctatgttcgaacaacgggtcttatagaaaataatttccaaatatgttcttcagttagcggtttcaaaaaaatgaatgttaacgatgtttacggtacgagctttcacaataaaaataaagaatcacgcatagtatcaattagcggtacaagttttgtaccaggtaaaacagccatagtgttccttatgccaatagggaatgatatgacagcttttagtattgtagagtcacgatatatctatgatctgatggatgttcttgatgatgagaatgatgattcattttctcttcgctttactgaagggcctgcaattattagtggtgtaaataataatgacaggttatatatgttaaaaaaagattttaattctgcgatcagtcaggctagtcttacagttactacgccgatgatttcattatatccctatgtcattagtaatgtattttacattcttccattatctattcttctttcttttatcctgtacttcttatggcagcgctggatatctcggaaaatgtccctggcagaagaaattaagaaaggaatgtcatccggagaattttctgtgcattatcaaccagtatgtgacacgacaaccaaagcgtgtctgggggttgaggcgttaatgcgttggcaacgcgaagatggtaaaaatatatctccggtagtttttatccgcgctgcagaagaggaaaatttaattataccgttaactaagcatctctttgaactgattattcaggatgtacaaagctggaaagtgaaaaagccgtttcaccttgggataaatattgccgctgaacatcttgcacatcctgattttgttgctgatgttttacatataaaaaatgcaatatctgataaatttaacattgtccttgagattaccgaacgcaatctggttgaagatactgatcatgcattacagaaaataaatgaattaaggagccatggctgtgaatttgcagttgatgattttggcacgggatattgttcccttggccttctgcaaaagttgtctgttgattatcttaaaatagataaaagctttatagacacgctcactacagcggaagacgaaacgcctgttttagatattattattaagctgagtaatcgactcaatttgataacgattgcagaaggtgtcagtacctctcaccaggcggaatatcttattcataataacgtgacattagttcagggatacttatatgcaaaaccaatgaaagcgacagagttttatcaatggtatgtaaacagataa

SEQ ID NO: 26 - DNA, 3807 bp, Escherichia coli
atgataaatttaagtaaggaagcaacggtggggaaagcattaacccctattgctatacttatgatgttgtcttttcctgtagcttctcaagcggcgggattagtcataaaaaatggaacggtatataacgccaatggtgtgccagtcgttgacatcaacaaacctaacggtagcggtttatctcataatatctgggataacctaaacgttgataaaaatggtgtcgttttcaataatagcgctaatgaatccagtacttcacttgccggaaatattcagggaaacagtaatctgacctccgggtcggcgaaggtgatcctgaatgaggttacttccaaaaatccttcaaccattaatgggatgatggaagttgcaggggataaagcggatctgattattgccaacccgaatggtattactgtaaacggtggcggttcaatcaatacaggtaaacttaccttaaccaccgggacgccggatatccaggatgacaagctggccggttactccgtgaacggcggtaccattacgctcggtaaactggataacgccagcccgacagaaattctgtcccgtaacgtggtagttaacggcaaagtgtctgccgatgagctgaacgttgttgctggcaataactatgttaatgccgcaggccaggtgaccggtagcgtatccgccacggggtcccgtaacggttacagcgtagatgttgccaaactgggcggaatgtatgcgaacaaaatcagtctggtcagcaccgagaaaggtgtgggggttcgcaacctcggcgttattgctgggggtgttaatggtgtcagcatcgattccaaaggtaacctgttaaacagtaacgcccagattcagtctgcaagcacgatcaacctgacaacaaatggtactctggataacaccaccggtacggtgacatctgtaggcactatctcgcttaataccaacaagaatactatcgtgaatacccgtgcgggtaacatctctacgatgggcgatatctacgttaacagcggtacgattgacaatactaacggcaagcttgcggctgcaggaatgctggcggttgataccaataacgccacgctgattaactctggtaaagggagttctgtcgggattgaagcggggctcgtggcgctgaaaaccggaacgctcaacaacagcaatggtcagattcgcggtggctatgtgggtcttgaatccgctgcgctgaataacaacaacggtgatatccagaccaccggcgatatcgccattatcagtaacggtaatgtggataacaacaaaggtctgatccgttcgtccaccgggcatatcgttattggcgcggcaggtagcgtaaataatggttcaaccaaaaccgccgataccggcagttctgactctctgggcattattgcagataccggcgtagaaattggtgcgaacaacatcaataacaacggcggacagattgcgtctaatggcaacgtctccctgtcaagttacagcacgatcgacgactatgcgggcaaaattctgtccaacagcaaagtgattatcaagggaagctctctgcgtaacgataccggggggatcagcggtaagcagggtattgaagtcgccgttggcggcagcctgaccaataatattggcgtgatcagctctgaagagggtgatatctccctgttagccaactccgtggataaccacggcggcttcatgatggggcagaacatcacgatggagtcgatgtctggcgtcaataacaacacagcgctgatcgtggccagcaaaaaactgaagataaatgcgcgcggcagtatcgaaaaccgcgatggcaataacttcggtaatgcttatggtctgtacttcggcatgcctcagcaaacgggtggaatggtcggcaaggaaggcatcgagctttccgggcagaacatctataacaacaacagccgtcttatcgctgaggatggtcctctgactctgcaggcgcagaacacgttcgacaacacgcgtgctctggtcaccagcggggcggatgcatctattcaggttggcggaacgtattacaacaactacgctaccacctggagtgcgggcaacctggatatcgacgcgaccacgctgcaaaacagcagcagcggtacgatgatcgataacaatgcgaccgggttcatagcatctgataaaaacctgtcactggaagtggtgaatagccttaccaactacggctggatcagcggtaaaggcgatgttgatgtcacggtgaataacggcaacctgtataaccgcaataccattgcggctgaaaaggggctggatattgccgcgttgaacggtattgaaaactggaaggatatttctgctggcggcgacctgacgatgaacaccaatcgccatgtgaccaacaactccaacagcaatatggtggggcagaatattgttattaacgcggttaacgatatcaacaaccgtggcaacattgtcagtgacgctgacctgaacgtgacgaccaaaggcaacctgtataactatctctatatggtagggtatggggatatcgcattgtcggcaaatagcgtggcgaacaataacgcgaccatcgaagcgacaggcgatctgattatcgattcgaagggtaacgtgggtaacaaccgcggtaatctgcatgcgttgaacggcgtgttgtctgttaaaggcaacaatctgaacaacgataacggtgaaattcgtggttatggcgatgtcacgctggcactgacgggcaactacgacagctataagggttcgctgacctctgaaacgggcgacgtgactctgacggcgaacattgtagacaacgcctatggtttgattgccggtgagaatgtttctgtcgatgctaaatcgacgatttacaacaacactgcgctgatcgcggcgaataaaaagctggttattaacgctggcggcaacctcgaaaaccgcgacgggaataacttcctgcgtaataacggcgcgctgtttggaattaccgacaacgttggcggcatcgtaggtaaagaaggtgtcacgctttctgctcagaacgtctacaacaataacagcagcatcatcgctgaaaatggtccgcttaatctgctgtccaggggaacgctggataatacccgcgcgcttcttagcagtggggctgatgccatcatccgtgcggcagggacgttctacaacaactatgccaccacgtacagcgccggtaatctcgacgtttatgcggcgtcgttgaacaacgccagcgatggtcgcctggaagacaataccgccacgggcgtgattgcgtctgacaaaaacctggatctgagcgttgataacagtgtcactaactatggttggatcagcggtaaaggagatgtgcatttcaatgttctgaaaggcacgctgtataaccgtaatgccatcgcggcggacaacgcgctgaccattaatgccctgaacggtgttgagaactttaaagacattgtggcgggtactgcgctgactattgatacgcagaagtatgttaccaacaacagcaacagtaatatgttgggacaaaccatcgcgatcaatgccgtgaatgacattaataaccgtggaaatattgtgggtgattattctctgggtgttaaaaccaccggtaatatttataactacctcaatatgctgagttatggtgtcgctggcgtatcggcaaataaggttacgaatagcggtaaagacgctgttctcggtggcttctacggtttagcgttagaagcaaacgaaactgataacaccggtactattgtcggcatgtaa

SEQ ID NO: 27 - DNA, 552 bp, Escherichia coli
atgaccgcgtattggctggcccagggcgtgggtgtcatcgcctttctgattggtatcacaacatttttcaatcgtgacgaacgtcgcttcaaaaagcagctttcggtctatagcgccgttattggcgtacatttttttcttctgggcacctatcccgctggtgccagtgccatccttaatgccattcgtacattgattaccttacgcacgcgcagcttatgggtaatggcgatttttattgtgctgactggcggaattggcctcgcgaagttccatcatcctgtcgaactattgccggttatcggtacgattgtcagtacctgggcgctgttccgctgtaaagggctcaccatgcgctgcgtgatgtggttttcaacgtgttgctgggtgattcacaacttctgggcggggtcgataggcggcacgatgattgagggtagttttctgcttatgaatggcctgaatatcattcgtttctggcggatgcagaaaaggggaattgatccgtttaaagtagagaaaacccctcccgccatagacgaaaggggttaa

SEQ ID NO: 28 - DNA, 2517 bp, Escherichia coli
atgccacaacgacaccaccagggacataaacgcacaccgaaacagttggcgctcattatcaaacgctgtttgccgatggtgctcactggcagcggcatgctttgcactaccgctaacgccgaagagtattatttcgaccccattatgctggaaaccacaaaaagtggtatgcaaacaaccgatctgtcacgtttttcaaaaaaatacgcacaactaccaggaacttatcaggttgatatctggctgaataaaaagaaggtttcacagaaaaaaattacatttaccgccaatgcagagcaacttctgcagccacagtttacggtagaacaactacgtgagctgggtattaaggtggatgaaatcccggcgctggctgaaaaagatgacgatagcgtgatcaactcgcttgaacaaatcattcccggtacagctgctgaatttgatttcaatcatcagcgacttaatttgagcattccccaaattgcactgtaccgtgatgcaagaggttacgtctccccttctcgttgggacgatggtataccaacgctgtttaccaactactcgtttacaggttctgataaccgttaccgccagggcaatcgtagccaacgacagtacctaaatatgcaaaatggtgccaattttggcccctggcgattacgtaactattctacgtggacacgcaacgatcaggcgtcaagctggaacactatcagtagttatttacaacgtgatatcaaggcgttgaagtctcagttgcttctgggagaaagcgccaccagcggcagtattttttccagctacaactttactggcgtgcaactcgcttccgacgataatatgttgccaaacagccagcgcggatttgccccaacggtacgcggtatcgcaaacagtagtgcaatcgtgactatcaggcaaaatggttatgtgatctatcaaagcaacgtgccagcgggtgcctttgaaattaacgatctctacccctcttccaacagcggcgatttagaagtcacgattgaagaaagtgacggtacgcaacgtcgctttatccagccttattcttcattacccatgatgcagcgacctgggcatctaaaatatagcgcgaccgctggacgctatcgcgctgatgcaaacagtgatagcaaggaacccgaatttgctgaagccacggcaatatatggtttgaataatacttttacgctgtatggcggcctgctcggttctgaagattattatgcgctggggatcggtatcggcggcacacttggcgcactgggcgcgttgtcgatggatatcaacagagctgacacccaattcgataaccagcactcttttcatggctatcaatggcgtacgcagtacatcaaagatatcccggaaaccaacaccaatatcgctgtcagctactatcgctataccaacgatggctattttagttttgatgaagccaatacccgcaattgggactataacagtcgccaaaaaagtgaaattcaattcaacatcagccagacaatatttgatggggtaagtctgtatgcctccggttcacagcaagactattggggcaataacgagaaaaacaggaatatctctgttggggtttccggccagcaatggggaattggttacagcctgaattatcaatacagccgctacactgatcaaaataatgaccgcgcactctctttgaatctcagtattccgttagaacgctggttaccgcgtagccgggtttcctatcagatgaccagccagaaagatcgcccaacccaacatgaaatgcgtcttgatggctcactgctggatgatggtcgcctgagctatagtctggaacaaagtctggatgacgataacaaccataacagtagcgtgaacgccagttaccgttcaccttatggaaccttcagtgccggatacagttacggtaatgacagtagccaatacaattacggcgttaccggcggcgtggttatccatcctcatggtgtgacgctctcgcaatatctgggcaacgcttttgcgcttattgatgctaacggggcatctggcgtgaggatacaaaactatccggggattgctactgatccctttggctatgcagtggttccttatctcacaacttatcaggaaaaccgtctctcggtagatactacgcagctgcccgataacgtcgatcttgaacaaacaacacagtttgtggtgcccaacagaggtgcaatggtagcggcgcgtttcaacgccaatatcggttatcgcgtacttgttacagtcagcgatcgcaacggtaaaccgttgccctttggcgctcttgccagcaacgatgatacggggcaacaaagtatcgtcgatgagggcggcatactatatctctctgggatatcgagtaaatcacaaagctggactgtacgctggggaaatcaggcagatcaacaatgtcagtttgcttttagtacaccggattcagaaccaacaacctctgtattacaaggcacagcgcagtgccattaa

SEQ ID NO: 29 - DNA, 1458 bp, Escherichia coli
atgacgatgcagcttgaagtaattctaccgctggtcgcctatctggtggtggtgttcggtatctcggtttatgcgatgcgtaaacggagcaccggcaccttccttaatgagtatttcctcggcagccgctctatgggcggtattgtgctggcgatgacgctcaccgcgacttatatcagtgccagttcgtttatcggcgggcctggagctgcttacaaatacggccttggctgggtattgctggcgatgattcagcttccggcagtctggctttcactcggtattctcggcaagaagtttgcgattcttgcgcgccgctacaatgcagtgacgctgaacgatatgctgtttgcccgctaccagagtcgtcttctggtgtggctggcgagtttgagtttgctggttgcgttcgttggtgcgatgaccgtgcagtttatcggcggtgcgcgcctgctggaaaccgcggcgggtattccttatgaaaccgggctgctgatttttggtatcagcattgcgttatataccgcctttggtggctttcgcgccagcgtgctgaacgacaccatgcaagggcttgtgatgctgattggcaccgttgtgctgcttattggcgtggtacatgccgctggtggcttaagcaacgcagtacagaccttgcaaaccatcgatccgcaactggttacgccacaaggcgctgacgatattctgtcgcctgcctttatgacgtcgttctgggtactggtgtgctttggcgttataggcctgccgcatactgcggtgcgctgtatctcttataaagacagcaaagcagtacaccgggggatcatcatcggtacgattgtagtcgcaatcctgatgttcggtatgcacctagccggagcgttaggtcgtgcagtgatccccgatctcaccgtaccggatctggtgatcccgacgttaatggtaaaagtgctgccaccgtttgctgccgggatctttctggctgcacctatggctgcgatcatgtcgacaattaacgcccaattgctacaaagttccgctacgatcattaaagatctctatctgaatatccgtccggatcaaatgcaaaacgagacgcgtctgaagcggatgtcggcggtaattacgttagttctcggcgcgttgctgctgcttgccgcctggaagccgccagaaatgatcatctggctgaatttgctggccttcggtgggctggaagccgttttcctgtggccgctggtgctgggtctttactgggaacgcgccaacgccaaaggcgcgctaagtgcgatgatcgttggcggcgtgctgtatgccgtactcgcgacgctgaatattcagtacctgggcttccaccctatcgtgccctcgttactactaagtttgctggctttcctggtcggaaaccgtttcggtacatccgtcccgcaagctaccgttttgactactgataaataa

SEQ ID NO: 30 - DNA, 1245 bp, Escherichia coli
atggcgatgcctttatcgttattgattggcctgcgttttagccgcggacggcgacgcggcggcatggtgtcgctgatctccgtcatttctaccattggcattgcccttggcgtggcggtattgatcgtcggcttaagcgcgatgaacggctttgaacgcgaactgaataaccgcattctggcggtggtgccgcatggcgaaatagaggcggtggatcaaccgtggactaactggcaggaagcactggataacgtgcaaaaagtgccaggtattgccgccgctgcgccgtatatcaatttcaccgggctggtggaaagtggagcgaatctgcgcgcaatccaggtgaagggcgttaacccgcaacaggaacagcgtctgagcgcattaccctcgtttgttcagggggatgcctggcgcaattttaaagcgggcgaacagcaaattatcatcggcaaaggcgtggcggatgcgctgaaagtgaagcagggcgattgggtgtcgattatgatccccaactcgaatcctgagcataaactgatgcagccaaaacgtgtgcgtttgcacgttgccggtattttgcagttgagtggtcaactcgatcacagttttgccatgatcccgctggcggatgcccaacaatatcttgatatgggttccagcgtgtcaggtattgcccttaaaatgacggatgttttcaacgccaataagctggtacgtgatgcgggtgaagtgaccaacagctatgtttatattaaaagctggattggtacttacggctatatgtatcgcgatatccagatgatccgcgccattatgtatctggcgatggtactggtgattggcgtggcctgtttcaacatcgtctccaccttagtgatggcggtgaaagacaagagtggcgatatcgcagtattaagaacgctgggggcgaaagatggtttaattcgcgccatctttgtctggtatggattgctggcagggctattcggtagcctgtgtggtgtgattatcggcgtagttgtttcactgcaacttaccccgattattgagcggattgaaaagctgatcggtcatcagttcctctccagcgatatctattttattgacttcctgccatcggaattgcactggctggacgtcttctacgtactggtcacagcattgttgctgagtcttttggcaagttggtatccggcgcggcgcgccagtaatattgaccctgcgcgagtccttagcggccagtaa

SEQ ID NO: 31 - DNA, 621 bp, Escherichia coli
atgagctataccctgccatccctgccgtatgcttacgatgccctggaaccgcacttcgataagcagaccatggaaatccaccacaccaaacaccatcagacctacgtcaacaacgccaacgcggcgttggaaagcctgccagaatttgccaacctgccggttgaagagttgatcaccaaactggaccagctgccagcagacaagaaaaccgtactgcgcaacaacgctggcggtcacgctaaccacagcctgttctggaaaggtctgaaaaaaggcaccaccctgcagggtgacctgaaagcggctatcgaacgtgatttcggttccgtagataacttcaaagcagaatttgagaaagcggcagcttcccgctttggttccggctgggcatggctggtgctgaaaggcgataaactggcggtggtttctactgctaaccaagactccccgctgatgggtgaagctatttctggcgcatccggcttcccgattctgggcctggatgtgtgggagcatgcttactacctgaaattccagaaccgccgcccggactacatcaaagagttctggaacgtggtgaactgggacgaagcagcggcacgtttcgcggcgaaaaaataa

SEQ ID NO: 32 - DNA, 3741 bp, Escherichia coli
atgagtaaacttttggatcgctttcgctacttcaaacaaaagggcgatacctttgccgatggtcacggacaggtgatgcatagcaaccgcgactgggaggacagctatcgccagcgttggcagttcgacaaaatcgtgcgttccacccacggtgttaactgtacaggctcctgtagctggaaaatctacgttaaaaatggtctggtgacctgggaaatccaacagaccgactacccgcgcactcgccctgacctgcccaatcatgaacctcgcggctgcccgcgtggcgcaagttactcctggtatctttacagcgctaaccgcctgaaatacccgctcattcgtaaacgactgattgaactgtggcgcgaagccctcaaacaacacagcgatccggtactggcgtgggcatcgattatgaacgatccgcaaaagagcctgagctacaaacaagtgcgtgggcgcggcgggtttatccgctccaactggccggaactaaaccagctgattgccgccgctaacgtctggactatcaaaacctacggcccggatcgcgttgccggtttctcgccgatcccggcgatgtcgatggtttcttacgccgccggaacacgttatctgtcactattaggtggcacctgtttaagcttctacgactggtattgcgacctgccgcccgcctcgccgatgacctggggcgagcaaaccgacgtaccggaatctgccgactggtataactccagttacatcatcgcgtgggggtctaacgtaccgcagacacgtacgccggacgcccatttctttaccgaagtacgctacaaaggcactaaaaccatcgccattacccctgactactctgaagtggccaaattgtgcgaccagtggctggcaccaaaacaaggcactgatagcgccctggcgatggcaatgggccatgtgattttaaaagagtttcatctcgataatcccagcgactactttatcaactactgccgccgctatagcgacatgccgatgctggtaatgctggaacctcgcgacgatggtagctacgtacccgggcgcatgatccgcgcgtctgacctggtagatggactaggcgaaagcaataatccgcagtggaaaaccgtggcggttaataccgcaggtgaattggtagtgccgaatggttcgattggtttccgctggggagaaaaaggcaaatggaatctggaatccattgccgccggtaaggaaaccgaattgtcgttaaccctgctcggtcaacatgacgccgttgcaggcgtggctttcccctactttggcggcatcgaaaatccgcattttcgcagcgtcaaacacaatcctgtcctggtgcgtcaattacctgttaaaaacctgacattagccggtggcagcacctgtccagtggtcagcgtttatgatttggtactggcgaattacggcctcgatcgcgggctggaagatgaaaacagcgcgaaagattacgctgaaatcaaaccgtacaccccagcctggggtgagcaaattaccggcgtgccgcgccagtatattgaaaccatcgctcgtgaatttgccgatactgcccataaaacgcatgggcgctcgatgattatcctcggcgcaggtgttaaccactggtatcacatggacatgaactaccggggaatgatcaatatgctgatcttctgcggttgtgtcggacaaagcggtggcggctgggcacactatgtcggtcaggaaaaactgcgcccgcaaaccggctggttgccgctggcctttgcgctcgactggaaccgaccaccgcgccagatgaacagcacctcgtttttctacaatcattccagccaatggcgctatgaaaaagtctctgcgcaggagttactttcaccgcttgccgatgccagtaagtacagcggtcatctgattgattttaacgttcgcgccgaacgcatgggctggctaccttctgcgccgcagttggggcgtaacccgctcgggattaaagctgaagccgacaaggccggattatcccccacagaatttaccgcccaggcgctgaaatcgggcgatttacgtatggcctgcgaacaaccagatagcggcagcaatcatccgcgtaatttgtttgtctggcgttctaacctgcttggctcctccggcaaaggccacgagtatatgcagaagtatctgctggggaccgaaagcgggattcagggcgaggaactcggtgccagcgacgggatcaaaccggaagaagtcgagtggcaaactgcggcgattgaaggcaagctcgacctgctggtgacgctcgacttccgcatgtccagtacctgcctgttctccgatatcgttctgcccaccgccacctggtatgaaaaagacgatatgaacacctcggatatgcatccatttattcatccgctttctgcggcggtcgatccggcctgggaatcacgcagcgactgggaaatctacaaaggtattgccaaagcattttcgcaagtgtgcgtgggccatcttggcaaagaaaccgacgtggtattacaaccactgctgcatgactctccggcagagctctcacagccgtgtgaagtgctcgactggcgcaaaggcgaatgcgatctgagcccgggtaaaaccgcgccgaatattgtggcggtggagcgcgactaccctgctacgtatgaacgctttacctcgctcgggccattgatggacaaacttggcaacggcggtaaagggatttcgtggaatacgcaggatgaaatcgatttcctcggtaaactcaattacaccaagcgtgatggcccagcgcaggggcgtccgctgattgacaccgccattgacgcttcagaagtgattctggcactggcaccagaaaccaacggtcatgttgcagttaaagcgtggcaggcgctgggcgagatcaccggacgcgaacatacccatctggcgctgcacaaagaggacgagaagattcgctttcgcgatattcaggcgcagccgcgtaaaattatctccagccccacatggtctggtctggaaagcgatcacgtctcctataatgcgggatacaccaacgttcatgagttaattccgtggcgcacgctgtcgggacgccagcagctctatcaggatcatccgtggatgcgtgcttttggtgaaagcctggtggcatatcgcccgcctatcgacacccgtagcgtcagtgagatgcgccagatcccgccaaacggcttcccggaaaaagcacttaacttcctgacgccgcaccagaaatggggcattcactcaacctacagtgaaaacctgctaatgctgacgctctctcgcggtggaccgattgtctggatcagcgaaaccgatgcccgtgaactaaccattgtcgataacgactgggtggaagtgtttaacgccaatggcgcgctgacggcccgcgcggtggtcagccaacgtgtaccgccgggtatgaccatgatgtatcacgctcaggaacgcattatgaatattcctggttcggaagtaactggcatgcgcggcggtattcataactcggtcacccgcatttgcccgaaaccaacgcatatgattggcggttacgcgcagctggcctggggctttaactactacggcaccgtcggctcgaaccgcgacgaattcatcatgatccgcaagatgaagaacgttaactggctggatgatgaaggtcgcgatcaggtacaggaggcgaaaaaatga

SEQ ID NO: 33 - DNA, 2322 bp, Escherichia coli
atgaaaaaattaaccataggcttaattggtaatccaaattctggcaagacaaccttatttaaccagctcactggcgcacgtcagcgtgtaggtaactgggctggcgttaccgtcgaacgtaaagaagggcaattctccaccaccgatcatcaggtcacgctggtggacctgcccggcacctattctctgaccaccatctcatcgcagacctcgctcgatgagcaaatcgcctgtcactacattttgagtggcgacgccgacctgctgattaacgtggtggatgcgtctaaccttgagcgtaacctgtacctgacgctacaactgctggaactcggcattccctgcatcgtggcactgaacatgctcgacattgccgagaagcaaaatattcgtattgaaattgatgctctgtcggcgcgtcttggctgcccagtgatcccgctggtttcaacccgtggtcgcggtattgaagcgctcaagctggcgattgatcgctataaagctaacgagaatgtggaactggtgcattacgcacagccgctactcaacgaagcagattcactggcaaaagtgatgccttccgacatcccgctgaaacaacgtcgctggctgggcctgcaaatgctggaaggcgatatctacagccgcgcctacgccggtgaagcgtcgcagcatcttgatgccgccctcgcccgtctgcgtaatgagatggacgatccggcgctgcacattgccgatgcgcgttaccagtgcattgctgccatctgtgatgtggtaagcaacaccctgacggcagaacccagccgtttcaccactgcggtagataaaatcgtgctcaaccgtttcctcggtctgccgattttcctctttgtgatgtacctgatgttcctgctggctatcaacatcggcggggcgttacagccgctgtttgacgtcggctccgtggctctatttgtgcatggtattcaatggattggctacacgctccacttcccggactggctgactatcttcctcgcccagggcctgggtggcggtattaacaccgtgctgccactggtgccgcagatcggcatgatgtacctgttcctctccttccttgaggactccggctatatggcgcgtgcggcgtttgtgatggaccgcctgatgcaggcgctgggcttgccggggaaatcctttgtgccgctgatcgtcggtttcggttgtaacgtaccgtcggtaatgggtgcacgtacgcttgatgcaccgcgtgaacgtctgatgaccatcatgatggcaccgtttatgtcctgcggcgcgcgtctggctatcttcgcagtattcgcggctgccttcttcgggcagaacggtgcgctggcggtcttctcgctggatatgctcggtatcgtgatggcggtgctgactggcctgatgctcaagtacaccatcatgcgcggtgaagcgacgccgtttgtcatggagctgccggtctaccatgtaccacacgttaaaagcctgattatccagacctggcagcgtctgaaaggcttcgttctgcgtgctggtaaagtgattatcatcgtcagcattttcctgagcgctttcaacagcttctcgttgagcgggaaaatcgtcgataacatcaacgactcggcgctggcttccgtcagccgggtgatcaccccggtcttcaagccgattggcgtgcatgaagataactggcaggcaacagttggcctgtttacgggtgcaatggcgaaagaagtggtggtgggtacgctcaacaccctctacaccgcagaaaatattcaggacgaagagttcaatccggcggaatttaacctcggtgaagagttgttcagcgcggtagatgaaacctggcagagcctgaaagacaccttcagccttagcgtactgatgaaccccattgaagctagcaaaggcgacggcgaaatgggtaccggggcgatgggcgtgatggatcagaaattcggtagcgcagctgccgcatacagctacctgattttcgtcctgctgtatgtaccatgtatctcggtgatgggagccatcgcccgtgaatcaagccgtggctggatgggcttctccatcctgtgggggctgaatatcgcttactcactggcaacattgttctatcaagtcgccagctacagtcagcacccaacttacagcctggtgtgcattctggcggttatcctgtttaacatcgtggttatcggtctgctgcgccgcgcgcgtagccgggtggatatcgaactgctggcaacccgcaagtcggtaagtagttgctgcgcagccagcaccaccggtgattgccattaa

SEQ ID NO: 34 - DNA, 1110 bp, Escherichia coli
atgaaacggaatgcgaaaactatcatcgcagggatgattgcactggcaatttcacacaccgctttagccgacgatattaaagttgccgttgtcggcgcgatgtccggcccgattgcccagtggggcgatatggaatttaacggcgcgcgtcaggcgattaaagacattaatgccaaagggggaattaaaggcgataagctggttggcgtagaatatgacgacgcctgcgacccgaaacaagccgttgcggtcgccaacaaaatcgttaatgacggcattaaatacgttattggtcatctgtgttcttcttctacccagcctgcgtcagatatctatgaagacgaaggtattcttatgatctcgccgggagcgacaaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcaactgcccttgaacgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtga

SEQ ID NO: 35 - DNA, 1983 bp, Escherichia coli
atgtcacgtccgcaatttacctcgttgcgtttgagtttattggccttagctgtttctgccaccttgccaacgtttgcttttgctactgaaaccatgaccgttacggcaacggggaatgcccgtagttccttcgaagcgcctatgatggtcagcgtcatcgacacttccgctcctgaaaatcaaacggctacttcagccaccgatctgctgcgtcatgttcctggaattactctggatggtaccggacgaaccaacggtcaggatgtaaatatgcgtggctatgatcatcgcggcgtgctggttcttgtcgatggtgttcgtcagggaacggataccggacacctgaatggcacttttctcgatccggcgctgatcaagcgtgttgagattgttcgtggaccttcagcattactgtatggcagtggcgcgctgggtggagtgatctcctacgatacggtcgatgcaaaagatttattgcaggaaggacaaagcagtggttttcgtgtctttggtactggcggcacgggggaccatagcctgggattaggcgcgagcgcgtttgggcgaactgaaaatctggatggtattgtggcctggtccagtcgcgatcggggtgatttacgccagagcaatggtgaaaccgcgccgaatgacgagtccattaataacatgctggcgaaagggacctggcaaattgattcagcccagtctctgagcggtttagtgcgttactacaacaacgacgcgcgtgaaccaaaaaatccgcagaccgttggggcttctgaaagcagcaacccgatggttgatcgttcaacaattcaacgcgatgcgcagctttcttataaactcgccccgcagggcaacgactggttaaatgcagatgcaaaaatttattggtcggaagtccgtattaatgcgcaaaacacggggagttccggcgagtatcgtgaacagataacaaaaggagccaggctggagaaccgttccactctctttgccgacagtttcgcttctcacttactgacatatggcggtgagtattatcgtcaggaacaacatccgggcggcgcgacgacgggcttcccgcaagcaaaaatcgattttagctccggctggctacaggatgagatcaccttacgcgatctgccgattaccctgcttggcggaacccgctatgacagttatcgcggtagcagtgacggttacaaagatgttgatgccgacaaatggtcatctcgtgcggggatgactatcaatccgactaactggctgatgttatttggctcatatgcccaggcattccgcgccccgacgatgggcgaaatgtataacgattctaagcacttctcgattggtcgcttctataccaactattgggtgccaaacccgaacttacgtccggaaactaacgaaactcaggagtacggttttgggctgcgttttgatgacctgatgttgtccaatgatgctctggaatttaaagccagctactttgataccaaagcgaaggattacatctccacgaccgtcgatttcgcggcggcgacgactatgtcgtataacgtcccgaacgccaaaatctggggctgggatgtgatgacgaaatataccactgatctgtttagccttgatgtggcctataaccgtacccgcggcaaagacaccgataccggcgaatacatctccagcattaacccggatactgttaccagcactctgaatattccgatcgctcacagtggcttctctgttgggtgggttggtacgtttgccgatcgctcaacacatatcagcagcagttacagcaaacaaccaggctatggcgtgaatgatttctacgtcagttatcaaggacaacaggcgctcaaaggtatgaccactactttggtgttgggtaacgctttcgacaaagagtactggtcgccgcaaggcatcccacaggatggtcgtaacggaaaaattttcgtgagttatcaatggtaa

SEQ ID NO: 36 - DNA, 1041 bp, Escherichia coli
atgcagaacatcatccgaaaaggaggaactatgaaggctgcagttgttacgaaggatcatcatgttgacgttacggataaaacactgcgctcactgaaacatggcgaagccctgctgaaaatggagtgttgtggtgtatgtcataccgatcttcatgttaagaatggcgattttggtgacaaaaccggcgtaattctgggccatgaagggattggtgtggtggcagaagtgggtccaggtgtcacctcattaaaaccaggcgatcgtgccagcgtggcgtggttctacgaaggatgcggtcattgcgaatactgtaacagtggtaacgaaacgctctgccgttcagttaaaaatgccggatacagcgttgatggcgggatggcggaagagtgcatcgtggtcgccgattacgcggtaaaagtgccagatggtctggactcggcggcggccaacagcattacctgtgcgggggtcaccacctacaaagccgttaagctgtcaaaaattcgtcccgggcagtggattgctatctacggtcttggcggtctgggtaacctcgccctgcaatacgcgaagaatgtctttaacgcgaaagtgatcgccattgatgtcaatgatgagcagttaaaactggcaaccgaaatgggtgcagatttagcgattaactcacgcaccgaagacgccgccaaaattgtgcaggagaaaaccggtggcgctcacgctgcggtggtaacagcagtagctaaagctgcgtttaactcggcagttgatgctgtccgtgcaggcggtcgtgttgtggctgtcggtctgccgccggagtctatgagcctggatatcccacgtcttgtgctggatggcattgaggtggtcggttcgctggtcggcacgcgccaggatctaactgaagccttccagtttgccgccgaaggtaaagtggtgccgaaagtcgccctgcgtccgttagcggacatcaacaccatctttaccgagatggaagaaggcaaaatccgtggccgtatggtgattgatttccgccgctaa

SEQ ID NO: 37 - DNA, 1965 bp, Escherichia coli
gtgagcatggatataagcgatttttatcagacattttttgatgaagcggacgaactgttggctgatatggagcagcatctgctggttttgcagccggaagcgccagatgccgaacaattgaatgccatctttcgggctgcccactcgatcaaaggaggggcaggaacttttggcttcagcgttttgcaggaaaccacgcatctgatggaaaacctgctcgatgaagccagacgaggtgagatgcaactcaacaccgacattatcaatctgtttttggaaacgaaggacatcatgcaagaacagctcgacgcttataaacagtcgcaagagccggatgccgccagcttcgattatatctgccaggccttgcgtcaactggcattagaagcgaaaggcgaaacgccatccgcagtgacccgattaagtgtggttgccaaaagtgaaccgcaagatgagcagagtcgcagtcagttgccgcgacgaattatcctttcgcgcctgaaggccagcgaagtcgacctgctggaagaagagctggggcatctgacaacgttaactgacgtggtgaaaggggcggattctctctcggcaatattaccgggcgatatcgccgaagatgacatcacagcggtactctgttttgtgattgaagccgatcagattacctttgaaacagtagaagtctcgccaaaaatatccaccccaccagtgcttaaactggcagccgaacaagcgccaaccggtcgcgtggagcgggaaaaaacgacgcgtagcagtgaatccaccagcatccgtgtagcggtagaaaaggttgatcaattaattaacctcgtcggcgagctggttatcacccagtccatgcttgcccagcgttccagcgaactggacccggttaatcatggtgatttgattaccagcatggggcagttacaacgtaacgcccgtgatttgcaggaatcagtgatgtcgattcgcatgatgccgatggaatatgtctttagtcgctatccccggctggtgcgtgatctggcgggaaaactcggcaagcaggtagaactgacgctggtgggcagttccaccgagctcgacaagagcctgatagaacgcattatcgacccgctgacccacctggtacgcaatagcctcgatcacggtattgaactgccagaaaaacggctcgccgcaggtaaaaacagcgtcggaaatttaattctgtctgccgaacatcagggcggcaacatttgcattgaagtgaccgacgatggggcggggctaaaccgtgagcgaattctggcaaaagcggcctcgcaaggtttgactgtcagcgaaaacatgagcgacgacgaagtcgcgatgctgatatttgcaccaggcttctccacggcagagcaggtcaccgacgtctccgggcgcggcgtcggcatggacgtcgttaaacgtaatatccaggagatgggcggtcatgttgaaatccagtcgaagcagggtactggcactacgatccgcattttactgccgctgacgctggccatcctcgacggcatgtccgtacgcgttgcggatgaagtattcattctgccgctgaatgctgttatggaatcactgcaaccccgtgaagccgatctgcatccactggccggcggcgagcgggtgctggaagtgcggggtgaatatctgcccatcgtcgaactgtggaaagtgttcaacgtcgcgggcgcgaaaaccgaagctacccagggaattgtggtgatcttacaaagtggcggtcgccgctacgccttgctggtggatcaattaattggtcaacatcaggttgtagttaaaaaccttgaaagtaactatcgcaaagtccccggcatttctgctgcgaccattcttggcgacggcagcgtggcactgattgttgatgtctccgccttgcaggcgataaaccgcgaacaacgtatggcgaacaccgccgcctga

SEQ ID NO: 38 - DNA, 600 bp, Escherichia coli
atgaacaaacacaccgaacatgatactcgcgaacatctcctggcgacgggcgagcaactttgcctgcaacgtggattcaccgggatggggctaagcgaattactaaaaaccgctgaagtgccgaaagggtccttctatcactactttcgctctaaagaagcgtttggcgttgccatgcttgagcgccattacgccacatatcaccaacgactgactgagttgctgcaatccggcgaaggtaactaccgcgaccgcatactggcttattaccagcaaacactgaaccagttttgccaacatggaaccatcagtggttgcctgacagtaaaactctctgccgaagtgtgcgatctgtcagaagatatgcgtagcgcgatggataaaggcgctcgcggcgtgatcgccctgctctcgcaggctctggaaaatggccgtgatagccattgtttaaccttttgtggcgaaccgctgcaacaggcacaagtgctttacgcactatggctgggtgcgaatctgcaggccaaaatttcgcgcaattccgagccactggaaaacgcgctggcacatgtaaaaaccattattgcgacgcctgccgtttag

SEQ ID NO: 39 - DNA, 1551 bp, Escherichia coli
atgcaacgtcgtgatttcttgaaatattccgtcgcactgggtgtggcttcagccttgccactgtggagccgtgcagtctttgcggcggaacgcccaacgttaccgatccctgatttgctcacgaccgatgcccgtaatcgcattcagttaactattggcgcaggtcagtctacctttggcgagaaaactgcaactacctggggctataacggcaatctgctggggccggcggtgaaattacagcgcggcaaagcggtaacggttgatatctataaccaactgacggaagagacgacgttgcactggcacgggctggaagtaccgggtgaagtcgacggcggcccacagggaattattccgccaggtggcaagcgctcggtgacgttgaacgttgatcaacctgccgctacctgctggttccatccacatcaacatggcaagaccgggcgacaggtggcgatggggctggctggtctggtggtgattgaagatgacgagatcctgaaattaatgctgccaaaacagtggggtatcgatgatgttccggtgatcgttcaggataagaaatttagcgccgacgggcagattgattatcaactggatgtgatgaccgccgccgtgggctggtttggcgatacgttgctgaccaacggtgcaatctacccgcaacacgctgccccgcgtggttggctgcgcctgcgtttgctcaatggctgtaatgcccgctcgctcaatttcgccaccagcgacaatcgcccgctttatgtgattgccagcgacggtggtctgctacctgaaccggtgaaggtgaacgagctgccggtgctgatgggcgagcgttttgaagtgctggtggaggttaacgacaacaaaccctttgacctggtgacgctgccggtcagccagatggggatggcgattgcgccgtttgataagcctcatccggtaatgcggattcagccgattgctattagtgcttccggtgctttgccagacacattaagtagcctgcctgcgttaccttcgctggaagggctgacggtacgcaagctgcaactttctatggacccgatgctcgatatgatggggatgcagatgctaatggagaaatatggcgatcaggcgatggtcggaatggatcacagccagatgatgggccatatggggcacggcaatatgaatcatatgaaccacggcgggaagttcgatttccaccatgccaataaaatcaacggtcaggcgtttgatatgaataagccgatgtttgcggcggcgaaagggcagtacgaacgttgggttatctctggcgtgggcgacatgatgctgcatccgttccatattcacggcacgcagttccgtatcttgtcagaaaatggcaaaccgccagcggctcatcgcgcgggctggaaagataccgttaaggtcgaaggcaatgtcagtgaagtgctggtgaagtttaatcacgacgcaccgaaagaacgtgcttatatggcgcactgccatctgctggagcatgaagatacggggatgatgttagggtttacggtataa

SEQ ID NO: 40 - DNA, 582 bp, Escherichia coli
ttgaaacaacaagaagaacataacaataaaattgatctacttgaaaaacaacaagcacagctaaagagtcaactggaaactattcaaaaacaacaaactggcataataagcagtacaaagactttaactcacgtaattaaatccgttaaagatcaacaaaacacttttatttttaccgagtttaacccagcaaaaacgaagtatttcatcctgaacaatggttcggtggctttagcgggtcgagtgttatccattgacgccacagagaatggcagtgttattcatatttcactggtcaacttattaagtacacctatctcaaatattggttttaatgcgacatggggtggtgaaaaacctgtcgatgccaaagagtttgcccggtggcaacaattacttttcaacacatcaatgaaatccacattgaaattattaccaggtcaatggcaagacattaatttgaccctgaagggtgtatcgcccaataacctgggatatctgaagttagccatcaacatggaaaatattcagttcgacaatcttccctctgctgaaaatcggcagaaaagaagcaaaaaataa

SEQ ID NO: 41 - DNA, 1014 bp, Escherichia coli
ttggattttaaacatcttactcaatttaaagatataattgaactggacaagcgccccgttaaacttgatgaacgggaaacgtttaatgtctcatggggtattgatgagaactaccaggttggggctgcgatttcaattgcttcaattcttgaaaataataaacaaaacaaatttacctttcacataatcgctgattacttagacaaagagtatattgaattattatcacaattagcaacgaagtatcaaacagtaattaaattatatcatattgattctgagccattgaaggcgctacctcaatcaaatatctggccagtatctatttattatcgtttgctttcatttgattatttttctgcgcgattggattcattattatatcttgatgctgatatcgtctgtaagggttcattgaacgagttaatagcattagagtttaaagatgaatatggggcagtggtaattgatgtagatgctatgcaaagtaaaagcgctgagcgtttgtgtaatgaggattttaacggtagctattttaactctggtgtaatgtatattaatttacgggaatggttaaaacaaagactaacggaaaaattctttgatctattatcagatgagtcaattataaaaaaattaaagtacccggatcaagatattttaaacttaatgtttctacatcatgctaaaatattaccgagaaaatataattgtatttatactataaagtcagaatttgaagaaaaaaatagtgaatattacacccggtttattaatgatgacactgtcttcatacattatactggtataactaagccatggcatgattgggcgaactacgcctctgcagattattttcgtaatatttataatatatcaccatggagaaatataccttataaaaaagctgttaaaaaacatgagtacaaagaaaaatataaacacttgctttaccagaaaaaatttctcgatggtgtttttacagcaattaaatataatgttatgaaaggttaa

SEQ ID NO: 42 - DNA, 126 bp, Escherichia coli
atgtatggcagatcctctttcgccgaatggatatggacaatacttcttaatacgaatggtacggttatgcgggaagagactaacgcggttactgttcagctatttgtcccttgttcctttttataa

SEQ ID NO: 43 - DNA, 2535 bp, Escherichia coli
atggcctcttcgcatcttttcattacgcttgcatcgggcatatgtctgctctgttccatatctgcttttgcccgggatagcttgttcaaccccagattactggaactggatcatcctgcggataatattgatattcaccagttcaaccgttcgaataccttacctgcgggaacatacaaagttgatgtgatgatcaacggcatgctcttcgaacgccaggaagttaaattcgcccaggataaccctgatgctgaactccacccatgctacgtggcgataaaaaacgtgctggcgacctatggtataaaagttgatgcgataaaatctctggcgaatgttgatgacaaaacatgcgtaaatccagttccgctgatcgacggggctacctggttactggacgccagtaaacttgcattgaatattactattccgcaaatttatctcaacaatgcagttaatggttatatcagcccttcccgttgggatcaggggatcaatgccatgatgatgaattatgatttttcggcatcgcataccatccggtcaaattatgacgacgacgatgacagttattatctgaatttgcgtaatggtattaatttaggcgcatggcgttttcgtaattacagcaccctgaattcttatgacggtaatgtggactaccattccgtcagtaattacattcagcgcgacatcatggcattacgtagccagattatgattggcgatacctggacggcaagcgatgtatttgatagtacacaggtgcgtggcgtgcggctgtataccgatgacgatatgttgccctccagccagaacggctttgcgccagtggtacatgggattgcgaaaactaacgccacggtgatcatcaaacaaaacggctacgttatttatcaatcagccgtaccacagggcgcatttgccctcaccgacttaaacacgaccagtagcggcggcgatctcgatgtcactatcaaagaagaagatggcagcgagcagcactttattcagccatttacttcactggccattctcaagcgtgaaggtcagaccgatgtagaccttagcattggagaagtgcgcgacgaaagcggctttacgcctgaggtcttgcagttacaagcaatgcacggtttccctttgggaataactttgtatggcggaacacaattggcaaatgattacgcttctgccgcgctgggtattggtaaagatatgggggcgctgggcgcgatttcttttgacgtgactcatgcccgctcgcagtttgactacgacgataatgagagtggtcaatcgtatcgttttctctattccaaacgttttgaagacaccaataccacctttcgtctggtgggttatcgctactctatggaggggttctacaccctcaatgaatgggtgtcgcgacaggataatgattctgatttctgggtaacgggcaaccgtcgcagccgcttcgaaggcacctggacgcaatctttcacgccaggctggggcaatatttatttaacattcagtcgacaggaatactggcagaccgatgaggtcgaacgtttattacagttcggctataacaacaactggcgaaacatctcctggaacgtttcctggaactatacggactcgatcaagcgctcattgggcaaccatcatgatgataacaatgatgatttcggcaaagaacagattttcatgttctcaatgtcgataccgctatcgtgctggatggaagacagctacgtcaactattcgttaacgcaaaacaaccaccatgaaagcacgatgcaggtcggtctgaacggaacgatgctggaagggcgtaacctgtcttataacgtacaggaatcgtggatgcactctcctgatgactcctacagcggcaatgccggaatgacctatgacgggacttatggctcggtcaatggtagctattcctggagccgtgactcccaacattttgattatggcgccagaggcggcgtgctggtgcatagtgacggagtgaccttctcgcaggaactgggcgaaacggtggcattggtcaaagcgccgggcgcagaaggcctgtccattgaaaacgccaccgggatttctaccgactggcgtggttataccgtaaaaacgcagcttagcccgtatgacgaaaaccgcgtggcattgaacagcgactatttctccaaagccaatattgaactggaaaacaccgtcatcaacctggtaccaacgcgcggtgcggtggtgaaagccgaatttgtcacccatgtcggttatcgcgtgctatttaacgtccgccaggtcaacggtaaaccaataatgtttggcgcgatggcaaccgcctctctcgaaacgggcacagtcaccgggattgtcggtgataacggcgaactgtatctctccgggatgcctgaaaaaggcgagtttttattgagttggggacaagctgcggatgaaaaatgtaaggcggcctatcacatcacccataaacctgatgataccagcctggttcaaatggatgcgatttgtcgctaa

SEQ ID NO: 44 - DNA, 3051 bp, Escherichia coli
atgcaggtcagcagaaggcagttctttaagatctgcgctggcggtatggcaggcaccacggcagcggcactgggttttgcacccagcgtagcactcgcggaaacccggcagtataaactgctgcgcacccgcgaaacccgtaatacctgcacctattgttccgtaggctgtgggctgttgatgtacagcctcggtgacggagcaaaaaacgccaaagcatctatcttccatatcgaaggcgatccggatcacccggtcaaccgcggtgcactttgtccgaaaggcgctggcctggtggatttcatccactccgaaagccgtctgaagtttccggaataccgtgcgccaggttctgataaatggcagcaaatcagttgggaagaggcgtttgatcgcatcgccaaactgatgaaagaagaccgcgatgctaactacattgcgcaaaacgccgaaggcgtgactgttaaccgctggctctccaccgggatgctgtgtgcttccgcgtcgagtaacgaaaccggctatttaacgcaaaaattctcccgcgcgctgggtatgctcgcggtcgacaaccaggcgcgtgtctgacacggaccaacggtagcaagtcttgctccaacatttggtcgcggtgcgatgaccaaccactgggtcgacatcaagaacgccaacctcgtcgtggtgatgggcggtaacgccgctgaagctcacccggtcgggttccgctgggcgatggaagccaaaattcacaatggcgcgaagctgattgtgatcgatcctcgctttacgcgtacggctgcggtggctgactactatgcccctattcgttccggtactgacattgctttcctgtcaggcgtattgctgtacctgctgaacaatgaaaaattcaaccgcgaatacactgaagcctataccaacgccagcctgatcgtgcgtgaggattacggctttgaagatggcctgttcaccggctacgacgcggaaaaacgcaagtacgataaatcctcctggacttatgaactggacgaaaacggcttcgccaaacgcgataccacgctgcaacatccgcgctgcgtgtggaacttgctgaaacagcacgtttcccgctacacgccagatgtggttgaaaacatctgtggtacgccaaaagacgcgttcctgaaagtctgcgaatacatcgcagaaaccagtgctcacgataaaactgcctcgttcctgtatgccctcggctggacgcaacactccgttggtgcgcaaaacattcgtacgatggcgatgatccagctgctgctcggtaacatggggatggctggcggcggcgttaacgccctgcgcggtcactccaatattcaggggctgacggacctggggctgctgtcgcagagcctgccaggttacatgacgctgccaagcgagaagcagaccgatctgcaaacctaccttaccgccaacacgccaaaaccgctgctggaaggccaggaaaactactggggcaactacccgaaattcttcgtctctatgatgaaggccttctttggtgataaagcgacggcagaaaatagctggggctttgactggttgccgaagtgggataaaggctacgacgtcctgcaatacttcgagatgatgaaagaaggcaaggtcaatggctatatctgccagggctttaaccctgttgcctcattcccgaacaaaaacaaagtgatcagttgtctgtcgaaactgaagttcctcgtcaccatcgacccgctgaacactgaaacctctaacttctggcagaaccacggtgagctgaacgaagttgactcgtcgaagatccagaccgaagtgttccgtctgccatcgacctgcttcgcagaagagaacggttcaatcgttaactcaggtcgctggttgcagtggcactggaaaggtgcggacgccccggggattgcgctgactgatggcgagatcctctccggtatcttcctgcgcttgcgcaagatgtatgccgaacagggtggcgcgaacccggaccaggtgctgaacatgacctggaactacgccattccgcatgagccgaagtcggaagaagtagcgatggagagcaacggtaaggcgctggccgatattaccgatccggcaaccggtgcggttatcgtcaagaaaggccaacaacttagctcgtttgcccaactgcgcgatgacggtacaacttcctgtggttgctggattttcgccggtagctggacgccggaaggcaaccagatggcacgccgtgataacgccgatccgtctggcctcggtaacacgctgggttgggcatgggcatggccgcttaaccgccgcattctgtataaccgcgcctccgcagatccgcagggtaacccgtgggatccgaagcgtcagttgctgaaatgggacggcactaagtggaccggctgggatattccggactacagcgcagcgcctccgggtagcggcgtcgggccgtttatcatgcagcaggaaggcatggggcgtctgtttgccctcgataagatggcggaaggtccgttcccggaacactacgagccgtttgaaacgccgctgggaactaacccgctgcatccaaacgttatctcgaacccggctgcacgtatctttaaagacgacgccgaagcattgggtaaagccgataagttcccgtatgtcggaaccacctatcgtctgaccgagcacttccactactggaccaaacacgcgctgttgaacgcgattttgcaaccagagcagtttgtggaaatcggggagtcgctggcgaataaacttggcattgcccagggcgataccgtgaaagtctcctccaaccgtggctatatcaaagccaaagcggtggtgaccaaacgtattcgcacgctgaaagcgaacggcaaagatatcgataccatcggtattcctattcactggggctatgaaggtgttgcgaaaaaaggctttattgccaatacgttgacgccattcgtcggtgatgcgaacacgcagacgccggagtttaagtccttccttgtgaatgtggaaaaggtgtaa

SEQ ID NO: 45 - DNA, 1686 bp, Escherichia coli
ttgaagaaggtttggcttaaccgttatcccgcggacgttccgacggagatcaaccctgaccgttatcaatctctggtagatatgtttgagcagtcggtcgcgcgctacgccgatcagcctgcgtttgtgaatatgggggaggtgatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgtcattgtgtccaactttgctcacacgctggaaaaagtggtcgataaaaccgcagttcagcacgttatcctgacccgtatgggcgatcagctttccacagcgaaaggcacactggttaacttcgttgttaaatacatcaaacgtctggtgccgaaatatcatctgccagatgccatttcatttcgtagcgccctgcacaacggctaccgtatgcagtatgtcaaacctgaactggtgccagaagatttagccttcctgcaatacactggcggcaccacaggtgtggcgaaaggcgcgatgctgactcaccgcaatatgctggcgaacctggaacaagttaacgctacgtatgggcctctgctgcatccgggcaaagagctggtggtgacggcgctgccgctgtatcacatttttgcactgaccattaactgcctgctgtttatcgaactgggtgggcagaacctgcttatcactaacccgcgggatattccggggctggtcaaagagctggcgaaatatccgtttaccgctatcaccggcgtaaacaccttgttcaacgcgttgctgaacaataaagagtttcaacaactggatttctccagtctgcacctttctgcaggcggtgggatgcctgttcagcaagtggtggcagaacgctgggtgaaactgaccggacagtatctgctggaaggttatggcctgaccgaatgtgcgccgctggtcagcgtgaacccctatgatattgattatcatagcggtagcattggtttgccggtgccgtcgacggaagccaaactggtggatgacgacgataatgaagtatcgccaggtcagccaggtgaactttgcgtcagaggtccacaggtgatgctgggctattggcagcgtccggatgctaccgatgaaatcatcaaaaacggctggttacacactggcgacatcgcggtaatggatgaagaaggattcctgcgcattgtcgatcgtaaaaaagacatgattttggtttccggttttaacgtctatcccaacgagattgaagatgtcgtcatgcagcatcctggcgtacaggaagtcgcggctgttggtgtaccttccggctccagtggtgaagcggtgaaaatcttcgtagtgaaaaaagatccatcgcttaccgaagagtcactggtgaccttttgccgccgtcagctcacgggctacaaagtaccgaagctggtggagtttcgtgatgagttaccgaaatctaacgtcggaaaaattttgcgacgagaattacgtgacgaagcgcgcggcaaagtggacaataaagcctga

SEQ ID NO: 46 - DNA, 618 bp, Escherichia coli
atgagtgcaatcgcgcctggaatgatcctcatcgcgtacctctgcggctccatttccagtgccattctggtttgccgcctgtgtgggctgcccgatccgcgaaccagcggctccggtaatcctggcgcaactaacgtgttacgtataggtggcaaaggagcagccgtagcagtactgattttcgacgttctgaaaggaatgttgcccgtctggggcgcgtatgaattaggtgtcagccccttctggctaggcttaattgccatcgccgcctgtcttggacacatctggcccgttttcttcggatttaaaggaggaaaaggcgttgctaccgcttttggtgccatcgcacccattggctgggatctcaccggagtaatggcgggaacctggttactgaccgtgctattgagcggatactcgtcgctgggagcgattgtcagtgcactgattgctccgttttatgtctggtggtttaagccacaattcaccttcccggtttcgatgctctcttgcctgatcctgctgcgtcatcatgacaacatccagcgtctgtggcgtcgtcaggagacaaaaatctggacgaaattcaaaagaaagcgcgaaaaggatcccgagtga

SEQ ID NO: 47 - DNA, 549 bp, Escherichia coli
atgaaaattaaaactctggcaatcgttgttctgtcggctctgtccctcagttctacagcggctctggccgctgccacgacggttaatggtgggaccgttcactttaaaggggaagttgttaacgccgcttgcgcagttgatgcaggctctgttgatcaaaccgttcagttaggacaggttcgtaccgcatcgctggcacaggacggagcaaccagttctgctgtcggttttaacattcagctgaatgattgcgataccaatgttgcatctaaagccgctgttgcctttttaggtacggtgattgatgcgggtcataccaacgttctggctctgcagagttcagctgcgggtagcgcaacaaacgttggtgtgcagatcctggacagaacgggtgctgcgctgacgctggatggtgcgacattcagtgagcaaacaaccctgaataacggtactaacaccattccgttccaggcgcgttattatgcaatcggcgaggcaaccccgggtgctgctaatgcggatgcgaccttcaaggttcagtatcaataa

SEQ ID NO: 48 - DNA, 1374 bp, Escherichia coli
atggcactttggggcgggcgttttacccaggcagcagatcaacggttcaaacaattcaacgactcactgcgctttgattaccgtctggcggagcaggatattgttggctctgtggcctggtccaaagccctggtcacggtcggcgtgttaaccgcagaagagcaggcgcaactggaagaggcgctgaacgtgctgctggaagatgttcgcgccaggccacaacaaatccttgaaagcgacgccgaagatatccatagctgggtggaaggcaaactgatcgacaaagtgggccagttaggcaaaaagctgcataccgggcgtagtcgtaatgatcaggtagcgactgacctgaaactgtggtgcaaagataccgttagtgagttactgacggctaaccggcagctgcaatcggcgctggtggaaaccgcacaaaacaatcaggacgcggtcatgccaggttacactcacctgcaacgcgcccagccggtgacgttcgcgcactggtgcctggcctatgttgagatgctggcgcgtgatgaaagccgtttgcaggatgcgcttaagcgtctggatgtcagcccgttaggttgtggcgcgctggcgggaacggcttatgaaatcgaccgtgaacagttagcgggctggctgggatttgcttcagcaacccgtaacagtctcgacagcgtttctgaccgtgaccacgtgctggaactgctttctgctgccgctatcggcatggtgcatctgtcgcgttttgctgaagatctgattttctttaacaccggcgaagcggggtttgtggagctttctgaccgcgtgacttccggttcatcattaatgccgcagaagaaaaacccggatgcgctggagctgattcgcggtaaatgcggtcgggtgcagggcgcgttaaccggcatgatgatgacgctgaaaggtttgccgctggcttacaacaaagatatgcaggaagacaaagaaggtctgttcgacgcgctcgatacctggttggactgcctgcatatggcggcgctggtgctggacggcattcaggtgaaacgcccgcgttgccaggaagcggcgcaacagggttacgcgaacgccactgaactggcggattatctggtggcgaaaggtgtgccgttccgcgaggcacaccatattgtgggtgaagctgtggtggaagccattcgtcagggcaaaccgctggaggagctgccgctcaccgagttgcagaaatttagtccggtgattggtgaagatgtctatccgattctgtcgctgcaatcgtgcctcgacaagcgtgcggcaaaaggcggcgtctcaccacagcaggtggcgcgggcgattgcttttgcaagggcgcggttggggtaa

SEQ ID NO: 49 - DNA, 243 bp, Escherichia coli
atgaaaaaagttctgtatggcatttttgccatatctgcgcttgcggcgacttctgcgtgggctgcacctgtacaggtgggcgaagcggcagggtcggcagcaacgtcggtttcggcggggagttcctccgcgaccagcgtcagcaccgtaagctcggcggtgggtgtcgcgctcgcggcaaccggtggcggtgatggttctaataccgggaccaccacaaccacgaccaccagtacccagtaa

SEQ ID NO: 50 - DNA, 1503 bp, Escherichia coli
atgacgaccgaccaacaccaggagatcctccgcaccgaaggattaagtaaatttttccccggcgtcaaagcgttagacaacgttgatttcagcctgcgccgtggcgaaatcatggcgctgctcggtgaaaacggggcgggaaaatcaacgctaatcaaagcattaactggtgtataccacgctgatcgcggcaccatctggctggaaggccaggctatctcaccgaaaaataccgcccacgcgcaacaactcggcattggcaccgtctatcaggaagtcaacctgctacccaatatgtcggtcgctgataatctatttataggccgcgaacccaaacgcttcggccttctacgccgcaaagagatggaaaagcgcgccaccgaactgatggcatcttacggtttctccctcgacgtgcgcgaaccgctcaaccgcttttcagtcgcgatgcagcaaatcgtcgctatttgccgggctatcgatctctctgccaaagtgctgatcctcgatgaacccaccgccagtctcgacacccaggaagtggagttactgtttgacctgatgcgtcagttgcgcgatcgcggcgtcagcctgatctttgtcactcactttctcgatcaggtctatcaggtcagcgatcggatcaccgtcttacgcaacggcagtttcgtaggctgtcgggaaacgtgcgagctaccgcagatcgaactggtaaaaatgatgctggggcgcgagctggatacccacgcgctacagcgtgccgggcgaacattgttgagcgacaaacccgttgccgcgttcaaaaattacggcaaaaaaggaacgatcgcaccgtttgatctcgaagtacgccccggcgagatcgtcggtctggctggattgctgggatcaggacgtaccgaaaccgccgaagtgatcttcggtatcaaacctgctgacagcggcacggcgttgatcaaaggcaaaccgcaaaacctgcgatcgccacatcaggcttcggtacttggcattggcttctgcccggaagacaggaaaaccgatggcatcatcgctgccgcctcggtgcgggaaaatatcatcctcgctctccaggcccggcgcggctggctacgtcccatttcccgcaaagaacagcaagagattgccgaacgctttatccgccagcttggcattcgcacaccttcaactgaacaaccgattgaatttctctccggcggcaatcagcaaaaagtgttgctttcacgttggctactgacccgaccgcaatttctgatcctcgatgagccaacccgcggcattgatgttggtgcccacgccgagatcatccgcctgattgaaacgctatgcgccgatggtctggcgctgctggtgatctcctccgaactggaagaactggtgggctatgccgaccgggtgatcatcatgcgcgatcgcaaacaggtggcggagatcccgctggcagagctttccgttccggcgatcatgaatgccattgcggcgtaa

SEQ ID NO: 51 - DNA, 1179 bp, Escherichia coli
atgagtcatgatccacaaccgctgggcggcaaaatcatcagtaaaccggtcatgatttttggaccgttaatcgtcatctgtatgctcctgattgtgaagcgtctggtgttcggtctgggctctgtctctgacctgaacggcggcttcccgtggggcgtgtggatcgcgtttgacctgctgattggcaccggctttgcctgtggcggctgggcgcttgcgtgggcggtatacgtctttaaccgtgggcaataccatccgctggtgcgtccggcgctgttggcgagtctgtttggttactcactgggtggcttgtcgatcactatcgacgtgggtcgctactggaacctgccgtacttctacattccgggtcacttcaacgtgaactcggtactgttcgagacggcggtctgtatgaccatctacatcggcgtgatggctctggagtttgctccggcactgtttgaacgtctgggctggaaggtgtcgctacagcatctgaacaaggtgatgttcttcatcatcgcgctcggtgcgctgctgccgaccatgcaccagtcttcaatggggtcgctgatgatctcggcgggctacaaggtgcatccgctgtggcagagctatgaaatgttgccgctgttctcgctgctgacggcgttcatcatgggcttctcgattgtcatctttgaaggttcactggtgcaggcgggtctgcgtggcaacggtccggatgaaaagagcctattcgtcaagctgaccaacaccatcagtgtgttgctggcgattttcatcgtgctgcgctttggcgagcttatctatcgcgacaagctgtcgttagcgtttgccggtgacttctactccgtgatgttctggattgaagtcctgctgatgctcttcccgctggtcgttctgcgtgtggcgaagctgcgtaatgattcccgcatgctgttcctgtcagcactgagcgcgctgttaggttgtgcaacctggcgtctgacctattcgctggtggcattcaacccgggcggcggttacgcctacttcccgacctgggaagaactgttgatttctattggttttgtggctattgagatttgcgcttacatcgtactcattcgtctactgccgatacttcctcctttaaaacaaaacgatcataatcgtcatgaggcgagcaaagcatga

SEQ ID NO: 52 - DNA, 4008 bp, Escherichia coli
atgttagctcgttcagggaaggtaagcatggctacgaagaagagaagtggagaagaaataaatgaccgacaaatcttatgcgggatgggaattaaactacgccgcttaactgcgggtatctgcctgataactcaacttgtgttccctatggctgcggcagcacaaggtgtggtaaacgccgcaacccaacaaccagttcctgcacaaattgccattgcaaatgccaatacggtgccctacacccttggagcgctggaatcggcccaaagcgttgccgaacgtttcggtatttcggtggctgagttacgcaaactcaaccagtttcgtacgtttgctcgaggttttgataatgtccgccagggtgatgaactggatgtcccggcacaagttagtgaaaataatttaaccccgccaccgggtaatagcagtggcaaccttgagcaacagatagccagtacttcacagcaaatcgggtctctgctcgccgaggatatgaacagcgagcaagcggcaaatatggcgcgtggatgggcctcttctcaggcttcaggcgcaatgacagactggttaagccgcttcggtaccgcaagaatcacgctgggcgtggatgaagattttagcctgaagaactcccagttcgattttctccatccgtggtatgaaacgcctgataatctctttttcagtcagcatactctccatcgtactgacgagcgtacgcagattaacaacggcttgggttggcgtcatttcactcccacatggatgtcgggcatcaacttctttttcgaccacgatcttagccgttaccactcccgcgccggcattggcgcggagtactggcgcgactatctaaaattaagcagtaacggctatttgcgactgaccaactggcgcagcgcacctgaactggacaacgattatgaagcacgcccggccaatggctgggatgtacgcgcagaaggctggctacccgcctggccgcaccttggcggtaaactggtctatgaacagtattatggcgatgaagtggccctgttcgataaagatgatcggcaaagtaatcctcatgccataaccgctggacttaactatacccccttcccgctgatgaccttcagcgcggagcaacgccagggtaaacagggcgaaaatgacacccgttttgccgtcgattttacctggcaacctggaagcgcgatgcagaaacagcttgacccgaatgaagtcgatgcacggcgtagccttgcaggcagccgttttgatctggtggatcgcaacaacaacatcgttctggaatatcgcaaaaaagaactggttcgcctgaccctgacagaccccgtgacagggaagtcaggagaagtgaaatcactggtttcgtcgctacaaaccaaatatgccctgaaaggctataacgtcgaagccaccgctctggaagctgccggtggtaaagtggttacaacgggtaaagatattctggttaccctgccggcgtaccggttcaccagtacgccagaaaccgataacacctggccgattgaagtcaccgctgaagatgtcaaaggcaatttttcgaatcgtgaacagagcatggtagtcgttcaggctcctacgctaagccagaaagattcctcggtatcgttaagtagccagacgttgagcgcggattcccattcaaccgccacactgacttttattgcgcatgatgcagcaggtaatcctgttatcgggctggtgctctcgacgcgtcacgaaggtgttcaggacatcaccctttctgactggaaagataatggtgacggaagctatacccagatcctgaccacaggagcgatgtctggcacgctgacgctgatgccacagctgaacggtgtggatgcggctaaagcccccgccgtggtgaatatcatttctgtttcgtcatcccggactcactcgtcaattaagattgataaggaccgttatctctccgggaatcctatcgaggtgacggtagaactgagagatgaaaatgacaaacctgttaaggagcaaaaacagcaactgaataccgcagtcagcatcgacaacgtgaaacctggtgtcactacagactggaaagaaaccgcagatggcgtctataaggcaacctataccgcctataccaaaggcagtgggcttactgcgaagctgttaatgcaaaactggaatgaagatttgcataccgctggatttatcatcgacgccaacccgcagtcagcgaaaattgcgacattatctgccagcaataatggtgtgctcgccaatgagaatgcagcaaacaccgtctcggtcaatgtcgctgatgaaggaagcaacccaatcaatgatcataccgtcacgtttgcggtattaagcggatcggcaacttcctttaacaatcaaaacaccgcaaaaacggatgttaatggtctggcgacttttgatctgaaaagtagtaagcaggaagacaacacggttgaagtcacccttgaaaatggcgtgaaacaaacgttaatcgtcagttttgtcggcgactcgagtaccgcgcaggttgatctgcagaagtcgaaaaatgaagtggtcgctgacggcaatgacagtgccacaatgaccgcgacagttcgggatgcaaaaggcaacctgctcaatgacgtcaaggtcaccttcaatgtcaattcagcagcagcgaaactgagccaaaccgaagtgaatagccacgacgggatcgccacagctacgctgaccagtttgaaaaatggtgattatacggttacggcctctgtgagctctggttctcaggctaatcaacaggtgatttttatcggtgatcaaagtactgctgccctgaccctcagtgtgccttcaggtgatatcaccgtcaccaacacagctccgctacatatgactgcaaccttgcaggataaaaatggcaatccactaaaagataaagaaatcaccttctctgtgccaaacgacgtcgcaagtcggttctcgattagcaacagcggaaaaggcatgacggatagcaacgggactgcaatcgcctccctgaccggcacgttagcgggcacgcatatgatcacggctcgtctggctaacagcaatgtcagcgatacacagccaatgacgtttgtggcggataaagacagagcggttgtcgttctgcaaacatcgaaagcggaaatcattgggaatggcgtggatgagacgactctgacagcaacagttaaagatccttttgataacgtggttaaaaatctttcagtagtcttccgcacctcccccgcagacacgcaactgagtctgaacgcgcgtaatactaatgagaacggtattgccgaagttacccttaagggcacggttttgggtgttcatacagccgaagccatactgcttaacggcaacagagatacgaaaatcgtcaatattgcgcccgatgccagcaacgcgcaggtcaccctgaacatccctgcacaacaggtggtgacgaataacagtgacagcgtgcagctgacggcgacggtgaaagacccgtcgaatcatccggtggcgggaataacggtgaacttcaccatgccacaggacgtggcggcaaactttacccttgaaaataacggtattgccatcactcaggccaatggcgaagcgcatgtcaccctcaaaggcaaaaaagcgggcacgcatactgtgaccgccacgctgggtaacaataatgccagcgatgcgcaaccagtcaccttcgtggcggataaggacagcgcggttgtcgttctgcaaacatcgaaagcggaaatcattgggaatggcgtggatgagacgactctgacggcaacagtgaaagatccttttgataacgcagtaaaagatctacaggtcaccttcagtaccaaccccgcagatactcaacttagtcagagcaaaagcaatactaacgacagtggtgtggccgaagttacctttaagggcacgggttttgggtgttcatacagccgaagccacactgcctaa

SEQ ID NO: 53 - DNA, 717 bp, Escherichia coli
atgcagaccccgcacattcttatcgttgaagacgagttggtaacacgcaacacgttgaaaagtattttcgaagcggaaggctatgatgttttcgaagcgacagatggcgcggaaatgcatcagatcctctctgaatatgacatcaacctggtgatcatggatatcaatctgccgggtaagaacggtcttctgttagcgcgtgaactgcgcgagcaggcgaatgttgcgttgatgttcctgactggccgtgacaacgaagtcgataaaattctcggcctcgaaatcggtgcagatgactacatcaccaaaccgttcaacccgcgtgaactgacgattcgtgcacgcaacctgctgtcccgtaccatgaatctgggtactgtcagcgaagaacgtcgtagcgttgaaagctacaagttcaatggttgggaactggacatcaacagccgttctttgatcggccctgatggcgagcagtacaagctgccgcgcagtgagttccgcgccatgcttcacttctgtgaaaacccaggcaaaattcagtcccgtgctgaactgctgaagaaaatgaccggccgtgagctgaaaccgcacgaccgtactgtagacgtgacgatccgccgtattcgtaaacatttcgaatctacgccggatacgccggaaatcatcgccaccattcacggtgaaggttatcgcttctgcggtgatctggaagattaa

SEQ ID NO: 54 - DNA, 993 bp, Escherichia coli
atgagtcagaatacgctgaaagttcatgatttaaatgaagatgcggaatttgatgagaacggagttgaggtttttgacgaaaaggccttagtagaagaggaacccagtgataacgatttggccgaagaggaactgttatcgcagggagccacacagcgtgtgttggacgcgactcagctttaccttggtgagattggttattcaccactgttaacggccgaagaagaagtttattttgcgcgtcgcgcactgcgtggagatgtcgcctctcgccgccggatgatcgagagtaacttgcgtctggtggtaaaaattgcccgccgttatggcaatcgtggtctggcgttgctggaccttatcgaagagggcaacctggggctgatccgtgcggtagagaagtttgacccggaacgtggtttccgcttctcaacatacgcaacctggtggattcgccagacgattgaacgggcgattatgaaccaaacccgtactattcgtttgccgattcacatcgtaaaggagctgaacgtttacctgcgaacagcacgtgagttgtcccataagctggaccatgaaccaagtgcggaagagatcgcagagcaactggataagccagttgatgacgtcagccgtatgcttcgtcttaacgagcgcattacctcggtagacaccccgctgggtggtgattccgaaaaagcgttgctggacatcctggccgatgaaaaagagaacggtccggaagataccacgcaagatgacgatatgaagcagagcatcgtcaaatggctgttcgagctgaacgccaaacagcgtgaagtactggcacgtcgattcggtttgctggggtacgaagcggcaacactggaagatgtaggtcgtgaaattggcctcacccgtgaacgtgttcgccagattcaggttgaaggcctgcgccgtttgcgcgaaatcctgcaaacgcaggggctgaatatcgaagcgctgttccgcgagtaa

SEQ ID NO: 55 - DNA, 1605 bp, Escherichia coli
atgaaaaaaattgcaattgtgggtgccgggcctacggggatctacaccttattctcgcttctacagcaacaaactccactttctatttctatcttcgagcaggctgacgaggccggtgtcgggatgccatacagtgatgaggaaaactcaaaaatgatgctggcaaatattgccagtattgaaataccgccgatttattgtacgtatctcgaatggctacaaaagcaagaagccagtcatctccagcgttatggcgttaaaaaagaaaccttgcacgatcgtcagtttttaccgcgaattctgctgggcgaatatttccgcgatcaatttttacgattagtagaccaggcacgaaagcaaaaatttgcagtggctgtttatgaatcatgccaggttaccgatctgcaaattacaaatgctggcgtcatgctcgctacaaatcaggatttacccagcgagacgtttgatttagcggtgatcgccacgggtcacgtctggcctgatgaagaagaagcaacccgaacgtattttccaagcccgtggtcaggcttgatggaagcaaaggtcgatgcgtgtaacgtgggtattatgggaacatccttgagcggactggatgcggcaatggcagtggctattcagcatggttcgttcattgaagatgataaacaacacgtcgtttttcaccgcgataacgcaagtgaaaagctaaatattacgttaatgtcgcgcacgggtattttacccgaagccgatttctattgccctattccctacgagcccttacacatcgtcactgatcaggcattaaatgctgagattcaaaaaggcgaatatggccttttggatcgggtatttagattgatagtagaggaaatcaagtttgctgatccagactggagtcaacgcatagccttagagagcctgaatgtcgattcctttgctcaagcctggtttgccgagcgcaaacaacgcgaccaatttgactgggcagaaaaaaatctccaggaagtcgaacgcaataaacgagaaaaacatactgttccctggcgttatgtcattctgcgcctgcatgaagccgtacaggaaattgttccacatctgaatgaacacgaccataaacggttcagtaaaggccttgcccgggttttcatcgataattatgcggcaatcccttcagagtctattcgtcgcctacttgccttacgtgaagcgggaatcattcatattctcgctctcggtgaagactacaaaatggaaattaacgagtcgcgcaccgtcctgaaaacggaagacaacagctactcgtttgacgtttttattgatgcccgcgggcagcgtccgcttaaagtgaaagatattcctttccctggactacgcgaacaattacagaaaacaggggatgaaatccctgatgttggtgaagattatacgttacagcaacccgaagatattcgtgggcgcgtagcgttcggcgcgttgccctggttgatgcacgaccagcctttcgttcagggacttacggcatgtgcagaaattggtgaggcgatggctcgggcggtcgtaaagcctgcatcccgtgctcgtcggcgtctttcgtttgattaa

SEQ ID NO: 56 - DNA, 570 bp, Escherichia coli
atgccagcgcgacatttgtacttcattatgactaatacgtggaacagattggcgctcttgattttcgccgttttatcgttgctggtggcgggtgaattgcaggcaggagtggtggtcggcggaacgcgatttatctttccggcagacagagaatcgatatctattttactgactaataccagtcaggaatcctggcttattaaccgtaaaatcaaccgcccaacgcgttgggccgggggtgaagcgtcgacagtgccagcaccattactggccgctccgccacttattctcctgaagcccggtacgacaggcacgttgcgcttgctgagaacggaaagcgacatcttgcctgtggatcgcgaaacgctatttgagttaagcattgccagcgtgccatccggcaaagttgaaaatcagagcgtaaaagtggcgatgcgctcggtatttaaactgttctggcgacccgaaggttgccgggcgacccgctggaagcttatcaacaattacgctggacacggaagtcgcagggtgtacaactcactaacccaacgccttattacattaacctga

SEQ ID NO: 57 - DNA, 1191 bp, Escherichia coli
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccgggtggcactggcgcactacgcatagctgccgatttcctggcaaaaaataccagcgttaagcgagtgtgggtgagcaacccaagctggccgaaccataagagcgtctttaactctgcagatctggaagttcgtgaatacgcttattatgatgcggaaaaccacacccttgacttcgatgcactgattaacagcctgaacgaagctcaggctggcgacgtagtgctgttccatggctgctgccacaacccaaccggtatcgaccctacgctggaacaatggcagacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtgtatgctgttgcttctggtcgcgtaaacgtggccgggatgacaccagataacatggctccgctgtgcgaagcgattgtggcagtgctgtaa

SEQ ID NO: 58 - DNA, 609 bp, Escherichia coli
atgcgattgttgcgtttttgttgcgcattagatcacttaatttgctttacatctcccgtaaacacttttctgcgatacaatgcctttacgttatgtaacggagagttcggcatgtcacaccccgcgttaacgcaactgcgtgcgctgcgctattgtaaagagatccctgccctggagccgcaactgctcgactggctgttgctggaagattccatgacaaaacgttttgaacagcagggaaaaacggtgagcgtgacgatgatccgcgaagggtttgtcgagcagaatgaaatccccgaagaactgccgctgctgccgaaagagtctcgttactggttacgtgaaattttgttatgtgccgatggtgaaccgtggcttgccggtcgtaccgtcgttcctgtgtcaacgttaagcgggccggagctggcgttacaaaaattgggtaaaacgccgttaggacgctatctgttcacatcatcgacattaacccgggactttattgagataggccgtgatgccggactgtgggggcgacgttcccgcttgcgattaagcggtaaaccgctgttgctaacagaactgtttttaccgacgtcaccgttgtactaa

SEQ ID NO: 59 - DNA, 918 bp, Escherichia coli
atgcttttggccgggagtagtttactgacgttgctcgatgatatcgccacactgctggacgatatctccgtgatgggcaaactggcggcgaagaaaaccgccggtgtattaggggatgacttatcgctcaatgcgcaacaagtttcaggcgtgcgggccaaccgggaacttcccgtggtctggggcgtggcgaaaggatcgctgattaataaagtgattctggtgccactggcgctgatcatcagtgcgtttatcccgtgggcgattacgccattgttgatgatcggtggcgcgtttctctgctttgaaggggtagagaaagtgctgcatatgctggaggcgcgtaaacataaagaagatccggcgcagagccagcagcgtctggagaagctggcggcgcaggatccgctgaagtttgaaaaggacaaaataaaaggggcgattcgtaccgattttatattgtctgcggaaatcgtcgccatcacgctggggattgtggccgaagcgccgttgcttaatcaggtgctggtgctttcaggcatcgcgctggtagtgaccgtgggcgtctacggtctggtcggggttatcgttaagattgatgacctggggtattggctggcggaaaaatccagcgcgctgatgcaggcattaggtaaaggattattgattatcgcgccctggctgatgaaagcgttatcgattgtcggcacgctggcgatgttcctcgtcggcggcgggattgtggtacatggtattgcgccgctgcatcacgccattgaacatttcgccgggcagcaaagtgcagtggtggcgatgatattaccgactgttttaaatctgattcttggatttatcatcggcggcatcgtggtgctgggagtgaaagccgtagcgaaaatgcgcggtcaggtacattaa

SEQ ID NO: 60 - DNA, 861 bp, Escherichia coli
atgccattgatttctctcgctgaagggctggcacatgcccgcgaacatcactattcgttaggtgcgtttaatgtgctggactcgcactttttacgcgccttgtttgctgctgccagacaagaacgttcgccctttattatcaacattgccgaagtacattttaaatatatctcgctggattccctggtcgagacagtgaaatttgaagcggcgcgtcatgacattccggtggtgttaaatctcgatcacggactacattttgaatcggtggtgcaagcgttgcggctgggattcagctcggtaatgtttgatggctccacgctggggtatgaagaaaatgtccgccagacgcgggaagtggtgaaaatgtgccatgccgttggcgtatcggtagaagctgaactgggcgcggtgggtggcgacgaaggcggtgcactctatggtcatgccgatgaatccctgtttaccgatccgcaactggcgcgtgattttgtcgaccgtaccggcattgacgcgctggccgtggcgattggcaatgcacacggcaaatatcgcggcgaaccgaaactcgatttcccgcgcctcgacgctatccgccagcaggctgctatcccgctggtattacacggcggctcagggattagcgatgccgatttccgccgcgccatcgaactgggtatccacaaaatcaatttctacactggcatgtcgcaagcggcgctggctgccgttgaggcccgaatggcgcaccgtcagccgatgtatgacgagtttgccgagttactgctggcagtggaagccgccatcagcgatacggttgcacaacagatgcgtatttttggcagtgcggggcaggcatga

SEQ ID NO: 61 - DNA, 654 bp, Escherichia coli
atggcaagtcctataaacagcggaataatgatggcaaatctctgcccttccacctttttggataaaaatagaaatgtcgctgctgagcttgatattaaaaataacgagaaaaaatattcacctggcagtaatttcgcaaaatggatgttgcaggaaataaagcgcttaatactaaacatcatgtccggttctcggagtattaacaccgatattcttgactatttccaccctatgccaggcacggaaaataatggtaatcgtacctgggtggcggcaactggagaggatgaatatatcgaaatcaaacaaacaggtgataagtcttttaatattacactggttggcagagataaaccttcacgaaaagaaattccatattcaggagtggctgtagcgactattattaaatcgttgtctgaaaaaacttctgcgttagaaacacattccgctgacacggttttgaggaagaaattagttaatagtattgttatgaagaatacggattttaattacgaaattcctgcaggaattttaagtaatatatatgatttattaaaactgcgaattaaaaaagatgaagggtatgtacctgttcaggaaagttttaaaagaactgatgttttttttgattccatgattatggatgctcattga

SEQ ID NO: 62 - DNA, 1275 bp, Escherichia coli
atgacagataaacgcaaagatggctcaggcaaattgctgtattgctctttttgcggcaaaagccagcatgaagtgcgcaagctgattgccggtccatccgtgtatatctgcgacgaatgtgttgatttatgtaacgacatcattcgcgaagagattaaagaagttgcaccgcatcgtgaacgcagtgcgctaccgacgccgcatgaaattcgtaaccacctggacgattacgttatcggccaggaacaggcgaaaaaagtgctggcggtcgcggtatacaaccactacaaacgtctgcgcaacggcgataccagcaatggcgtcgagttgggcaaaagtaacattctgctgatcggtccgaccggttccggtaaaacgctgctggctgaaacgctggcgcgcctgctggacgtcccgttcaccatggccgacgcaaccacgctgaccgaagccggttatgtgggcgaagacgttgaaaacatcattcagaagctgttgcaaaagtgcgattacgacgtacagaaagcgcagcgcgggattgtctacatcgatgaaatcgacaagatttctcgtaagtcagacaacccgtctattacccgtgacgtttccggtgaaggcgtacagcaggcactgttgaaactgatcgaaggtacggtagctgctgttccaccgcaaggtggacgtaaacatccgcagcaggaattcttgcaggttgatacctctaagatcctgtttatttgtggcggtgcgtttgccggtctggataaagtgatttcccatcgtgtagaaaccggctccggcattggttttggcgcgacggtaaaagcgaagtccgacaaagcaagcgaaggcgagctgctggcgcaggttgaaccggaagatctgatcaagtttggtcttatccctgagtttattggtcgtctgccggttgtcgcaacgttgaatgaactgagcgaagaagctctgattcagatcctcaaagagccgaaaaacgccctgaccaagcagtatcaggcgctgtttaatctggaaggcgtggatctggaattccgtgacgaggcgctggatgctatcgctaagaaagcgatggcgcgtaaaaccggtgcccgtggcctgcgttccatcgtagaagccgcactgctcgataccatgtacgatctgccgtccatggaagatgtcgaaaaagtggttatcgacgagtcggtaattgatggtcaaagcaaaccgttgctgatttatggcaagccggaagcgcaacaggcatctggtgaataa

SEQ ID NO: 63 - DNA, 2547 bp, Escherichia coli
ttgtacctctatattgagactctgaaacagagactggatgccataaatcaattgcgtgtggatcgcgcgcttgctgctatggggcctgcattccaacaggtctacagtctactgccgacattgttgcactatcaccatccgctaatgccgggttaccttgatggtaacgttcccaaaggcatttgcctttacacgcctgatgaaactcaacgccactacctgaacgagcttgaactgtatcgtggaatgtcagtacaggacccgccgaaaggtgagcttccaattactggtgtatacaccatgggcagcacctcgtccgtagggcaaagttgttcctctgacctggatatctgggtctgtcatcaatcctggctcgatagcgaagagcgccagttgctacaacgtaaatgcagcctgctggaaagctgggccgcctcgctgggtgtggaagttagcttcttcctgattgatgaaaaccgttttcgccacaacgagagtggcagtctggggggcgaagattgtggctccacccagcatatactgctgcttgatgaattttatcgtaccgccgtgcgtctcgccggtaagcgtattctgtggaatatggtgccgtgcgacgaagaagagcattacgatgactacgtaatgacgctctacgcacaaggcgtgctgacgccaaatgaatggctggatctcggcggcttaagctcgctttccgctgaagaatacttcggtgccagcctttggcaactctacaagagtatcgattctccatacaaagcggtgctgaagacactgctgctggaagcctattcctgggaatacccgaacccacgcctgctggcgaaagatatcaaacagcgtttgcacgacggcgagattgtatcgtttggcctcgatccttactgcatgatgctggagcgtgttactgaatacctgacggcgattgaagatttcacccgtctggatttagtacgtcgctgcttctatttaaaagtgtgcgaaaagctcagccgtgaacgcgcctgcgtaggctggcgtcgcgcagtgttgagccagttagtgagcgagtgggggtgggacgaagctcgtctggcaatgctcgataaccgcgctaactggaagattgatcaggtgcgtgaggcgcacaacgagttgctcgacgcgatgatgcagagctaccgtaatctgatccgctttgcgcgtcgcaataaccttagcgtctccgccagtccgcaggatatcggcgtgctgacgcgtaagctgtatgccgcgtttgaagcattaccaggtaaagtgacgctggtaaacccgcagatttcacccgatctctcggaaccgaatctgacctttatttatgtgccgccgggccgggctaaccgttcaggttggtatctgtataaccgcgcgccaaatattgagtcgatcatcagccatcagccgctggaatataaccgttacctgaataaactggtggcgtgggcatggtttaacggcctgctgacctcgcgcacccgcttgtatattaaaggtaacggcattgtcgatttgcctaagttgcaggagatggtcgccgacgtgtcgcaccatttcccgttgcgcttacctgcaccgacaccgaaggcgctctacagcccgtgtgagatccgccatctggcgattatcgttaacctggaatatgacccgacagctgcgttccgcaatcaggtggtgcattttgatttccgtaagctggatgtcttcagctttggcgagaatcaaaattgcctggtaggtagcgttgacctgctgtatcgcaactcgtggaatgaagtgcgtacgctgcacttcaacggcgagcaatcgatgatcgaagccctgaaaactattctcggcaaaatgcatcaggacgccgcaccgccagatagcgtggaagtcttctgttatagccagcatctgcgcggcttaattcgtactcgcgtgcagcaactggtttctgagtgtattgaattgcgtctttccagcacccgccaggaaaccgggcgtttcaaggcgctgcgcgtttctggtcaaacctggggcttgttcttcgaacgcctgaatgtatcggtacagaaactggagaacgccatcgagttttatggcgctatttcgcataacaaactgcacggcctgtcagtgcaggttgaaaccaatcacgtcaaattaccggcggtggtggacggctttgccagcgaagggatcatccagttctttttcgaagaaacgcaagatgagaatggctttaatatctacattctcgacgaaagcaatcgggttgaggtgtatcaccactgcgaaggcagcaaagaggagctggtacgtgacgtcagtcgcttctactcgtcatcgcatgaccgttttacctacggctcaagcttcatcaacttcaacctgccgcagttctatcagattgtgaaggttgatggtcgtgaacaggtgattccgttccgcacaaaatctatcggtaacatgccgcctgccaatcaggatcacgatacgccgctattacagcagtatttttcgtga

SEQ ID NO: 64 - DNA, 939 bp, Escherichia coli
atgaaagtcgcagtcctcggcgctgctggcggtattggccaggcgcttgcactactgttaaaaacccaactgccttcaggttcagaactctctctgtatgatatcgctccagtgactcccggtgtggccgtcgatctgagccatatccctactgctgtgaaaatcaaaggtttttctggtgaagatgcgactccggcgctggaaggcgcagatgtcgttcttatctctgcaggtgtagcgcgtaaaccgggtatggatcgttccgacctgtttaatgttaacgccggcatcgtgaaaaacctggtacagcaagttgcgaaaacctgcccgaaagcgtgcattggtattatcactaacccggttaacaccacagttgcgattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgttgtcacaggttcctggcgttagttttaccgagcaggaagtggctgatctgaccaaacgtatccagaacgcgggtactgaggtggttgaagcgaaagccggtggcgggtcggcaaccctgtctatgggccaggctgctgcacgttttggcctgtctctggttcgcgcactgcagggcgaacaaggcgttgtcgaatgtgcctacgttgaaggcgacggtcagtacgcccgtttcttctctcagccgcttctgctgggtaaaaacggcgtggaagagcgtaaatctatcggtaccctgagcgcatttgaacagaacgcgctggaaggtatgctggatacgctcaagaaagatatcgccctgggcgaagagttcgttaataagtaa

SEQ ID NO: 65 - DNA, 471 bp, Escherichia coli
ttgcaatatataacgtttattgcatgttttttttcacatgaaaacatgaaatattcgacatttcatgatattaaccttgatatgtgtgagataaagaattgcaattttaacaattcagagatgaatttcatttcgtgcgtcgggacaaattttagcggttctacatttaacaatgtaaaaacaacaacagcgcaactaattaagacaccaacaaagtggacgaataatactctaaaatattggttctccagttgtaataaacgtaatatcatcttcacttttaatacaatatctgatagaaatatgaaattaaaaggtatcaaggatatattactgtcgttggttgaccaaaaagtcaatatttatagtgtaaggcaagagcttctgaattttcttaataatgatttatataaaaatgatggagagattctctcttataaagaatcaataatgttgttttgtgctgaatag

SEQ ID NO: 66 - DNA, 2805 bp, Escherichia coli
atgattactcatggttgttatacccggacccggcacaagcataagctaaaaaaaacattgattatgcttagtgctggtttaggattgtttttttatgttaatcagaattcatttgcaaatggtgaaaattattttaaattgggttcggattcaaaactgttaactcatgatagctatcagaatcgccttttttatacgttgaaaactggtgaaactgttgccgatctttctaaatcgcaagatattaatttatcgacgatttggtcgttgaataagcatttatacagttctgaaagcgaaatgatgaaggccgcgcctggtcagcagatcattttgccactcaaaaaacttccctttgaatacagtgcactaccacttttaggttcggcacctcttgttgctgcaggtggtgttgctggtcacacgaataaactgactaaaatgtccccggacgtgaccaaaagcaacatgaccgatgacaaggcattaaattatgcggcacaacaggcggcgagtctcggtagccagcttcagtcgcgatctctgaacggcgattacgcgaaagataccgctcttggtatcgctggtaaccaggcttcgtcacagttgcaggcctggttacaacattatggaacggcagaggttaatctgcagagtggtaataactttgacggtagttcactggacttcttattaccgttctatgattccgaaaaaatgctggcatttggtcaggtcggagcgcgttacattgactcccgctttacggcaaatttaggtgcgggtcagcgttttttccttcctgcaaacatgttgggctataacgtcttcattgatcaggatttttctggtgataatacccgtttaggtattggtggcgaatactggcgagactatttcaaaagtagcgttaacggctatttccgcatgagcggctggcatgagtcatacaataagaaagactatgatgagcgcccagcaaatggcttcgatatccgttttaatggctatctaccgtcatatccggcattaggcgccaagctgatatatgagcagtattatggtgataatgttgctttgtttaattctgataagctgcagtcgaatcctggtgcggcgaccgttggtgtaaactatactccgattcctctggtgacgatggggatcgattaccgtcatggtacgggtaatgaaaatgatctcctttactcaatgcagttccgttatcagtttgataaatcgtggtctcagcaaattgaaccacagtatgttaacgagttaagaacattatcaggcagccgttacgatctggttcagcgtaataacaatattattctggagtacaagaagcaggatattctttctctgaatattccgcatgatattaatggtactgaacacagtacgcagaagattcagttgatcgttaagagcaaatacggtctggatcgtatcgtctgggatgatagtgcattacgcagtcagggcggtcagattcagcatagcggaagccaaagcgcacaagactaccaggctattttgcctgcttatgtgcaaggtggcagcaatatttataaagtgacggctcgcgcctatgaccgtaatggcaatagctctaacaatgtacagcttactattaccgttctgtcgaatggtcaagttgtcgaccaggttggggtaacggactttacggcggataagacttcggctaaagcggataacgccgataccattacttataccgcgacggtgaaaaagaatggggtagctcaggctaatgtccctgtttcatttaatattgtttcaggaactgcaactcttggggcaaatagtgccaaaacggatgctaacggtaaggcaaccgtaacgttgaagtcgagtacgccaggacaggtcgtcgtgtctgctaaaaccgcggagatgacttcagcacttaatgccagtgcggttatattttttgatcaaaccaaggccagcattactgagattaaggctgataagacaactgcagtagcaaatggtaaggatgctattaaatatactgtaaaagttatgaaaaacggtcagccagttaataatcaatccgttacattctcaacaaactttgggatgttcaacggtaagtctcaaacgcaagcaaccacgggaaatgatggtcgtgcgacgataacactaacttccagttccgccggtaaagcgactgttagtgcgacagtcagtgatggggctgaggttaaagcgactgaggtcactttttttgatgaactgaaaattgacaacaaggttgatattattggtaacaatgtcagaggcgagttgcctaatatttggctgcaatatggtcagtttaaactgaaagcaagcggtggtgatggtacatattcatggtattcagaaaataccagtatcgcgactgtcgatgcatcagggaaagtcactttgaatggtaaaggcagtgtcgtaattaaagccacatctggtgataagcaaacagtaagttacactataaaagcaccgtcgtatatgataaaagtggataagcaagcctattatgctgatgctatgtccatttgcaaaaatttattaccatccacacagacggtattgtcagatatttatgactcatggggggctgcaaataaatatagccattatagttctatgaactcaataactgcttggattaaacagacatctagtgagcagcgttctggagtatcaagcacttataacctaataacacaaaaccctcttcctggggttaatgttaatactccaaatgtctatgcggtttgtgtagaataa

SEQ ID NO: 67 - DNA, 1191 bp, Escherichia coli
atgatgaaaactgttaatgagctgattaaggatatcaattcgctgacctctcaccttcacgagaaagattttttgttaacgtgggaacagactccagatgaactgaaacaagtactggacgttgccgcagcattaaaagcactacgtgctgaaaacatctcaaccaaagtctttaatagtggattaggtatttccgtattccgcgacaactccactcgtacccgcttctcttatgcttccgcgcttaacctgctcggccttgcacagcaagatctcgatgaaggcaaatcacaaatcgctcacggcgaaaccgtgcgtgaaaccgccaatatgatctccttctgcgccgacgctattggtattcgcgacgatatgtatctgggcgcaggcaacgcctatatgcgtgaagttggcgctgcacttgatgacggttacaagcagggtgtactgccacagcgtccggctttagtgaacctgcaatgcgatattgaccacccgactcagtcaatggctgacctggcatggttacgtgaacactttggttcactggaaaacctgaaaggtaaaaaaatcgccatgacctgggcctactctccaagctatggcaaaccgctctctgtaccacaaggcatcatcggtctgatgactcgcttcggtatggatgtcaccctggcccatccggaaggctacgacctgatcccggatgtagtagaagtcgcgaaaaacaatgctaaagcatctggcggtagcttccgtcaggtcaccagcatggaagaagcgttcaaagatgcagacatcgtttatccgaagtcatgggcaccttacaaagtgatggaacagcgtaccgaattgctgcgcgctaacgatcacgaaggcttaaaagcactggaaaaacagtgtctggcacagaacgcgcaacacaaagactggcattgtactgaagagatgatggaactgacccgtgatggcgaagccctgtacatgcactgcctgccagctgatatcagcggcgtatcctgcaaagaaggggaagtgactgaaggcgtattcgaaaaataccgtatcgctacctacaaagaagccagctggaagccttatatcatcgccgcgatgatcctgtcccgtaaatacgccaaaccaggtgcactgctcgagcaactgctgaaagaagcgcaagaacgcgtgaaataa

SEQ ID NO: 68 - DNA, 279 bp, Escherichia coli
atggaaagtaatttcattgactggcatcccgctgacatcattgcgggtttgcgcaagaagggaacatcaatggcggcggagtctcgcagaaatggtttgagttcctcaacgctggcgaatgcattatcgcgcccatggccgaaaggagagatgattattgcgaaagccctgggaactgacccctgggttatctggccatcacgctaccatgatccgcagccccatgagtttatcgacagaacgcagttgatgcgtagctacactaaaccgaaaaaatga

SEQ ID NO: 69 - DNA, 531 bp, Escherichia coli
atgaagttcaaacgattgctgcatagcggcatcgccagtttgagtctggttgcctgcggggtgaatgcggcgacggatcttggcccggcaggggatattcatttctccatcactatcaccactaaagcttgcgagatggaaaaaagcgatctcgaagtcgatatgggaacaatgacgctgcaaaaacctgcggcagtcggtacggtgttgagcaagaaagatttcaccattgaactcaaagagtgcgatgggatatccaaagcgaccgttgagatggacagtcagtcggacagcgatgatgattccatgtttgcccttgaggctggtggcgcaacgggtgttgcgttgaagatagaggacgataaaggaacgcagcaagttcccaaaggctccagcggaacgccgattgaatgggcgattgatggcgaaaccacgtcgcttcactaccaggcgagttatgtggtcgtcaacactcaggccactggtggcacagcgaatgcccttgtaaatttttccatcacctatgagtaa

SEQ ID NO: 70 - DNA, 579 bp, Escherichia coli
atggatacatcaaatgcaacatccgttgttaatgtgagtgcgagttcttcgacatcgacgatctatgacttaggtaatatgtcgaaggatgaggtggttaagctatttgaggaactcggtgtttttcaggctgcgattctcatgttttcttatatgtatcaggcacaaagtaatctgtcgattgcaaagtttgctgatatgaatgaggcatctaaagcgtcaaccacggcacaaaagatggctaatcttgtggatgccaaaattgctgatgttcagagtagcactgataagaatgcgaaagccaaacttcctcaagacgtgattgactatataaacgatccacgtaatgacataagtgtaactggtattcgtgatcttagtggtgatttaagcgctggtgatctgcaaacagtgaaggcggctatttcagctaaagcgaataacctgacaacggtagtgaataatagccagctcgaaattcagcaaatgtcgaatacattaaatctcttaacgagtgcacgttctgatgtgcaatctctacaatatagaactatttcagcaatatcccttggtaaataa

SEQ ID NO: 71 - DNA, 675 bp, Escherichia coli
atgcgaaattttatcttccttatggctttcttctgttcatctgtgtttgccacacaaattccagtacctgaatcacccaagtatgtgaatgacttaaccggcacattaacaaacagcgaagtaaacaccctgaccaaccagattaaagccctgacgcaaaaaaactacgcacagttggtcgtgttagtggttgaaacaacaggcgatgaaaccatcgagcagtacgcgacgcgggtattcgatagctggaaaccgggtgataaagaccgcgatgatggagttctactgttagtcgcctggcaggatcataccgtgcgtattgaaatcggttacggactggaaggtattattacggatgcgcagtccgggaaaattattcgcaacagtattattcctgcatttaaaaaaggcgacctggctggtggtttgcaaaagggaatcagcgatattgaaagtcgtctgacggggaataatttagcaacgataacaccgaccgatcaccctttgccctttagcggttggtgggcattgttagtctgggcgatagtactgacgtttatttctgccagaggatatatcaaaacgctgggagtaatttgcttcgcagctattgtactggcctttgttttacctatagcaggcttcagcggaagctggggagtactcgcaacatga

SEQ ID NO: 72 - DNA, 2016 bp, Escherichia coli
atggctgaactgactgcgcttcacacattaacagcgcaaatgaaacgtgaagggatccgccgcttgctggtgttgagcggggaagagggttggtgttttgatcatgcgcttaagttacgtgatgccttacctggcgactggctgtggatttcgccgcagccagatgctgaaaaccactgttctccctcggcactacaaactttacttgggcgcgagttccggcatgcggtattcgacgcccgccacggctttgatgccgctgcctttgccgcacttagcggaacgttgaaagcgggaagctggctggttttgttactccctgtatgggaagagtgggaaaaccaacctgatgccgactcgctgcgctggagtgattgccctgaccctattgcgacgccgcattttgtccagcatttcaaacgcgtacttacggcggataacgacgctatcttctggcggcaaaaccagccgttctcgttggcgcattttactccccgtactgactggcaccccgcgaccggcgcaccacaaccagaacaacagcaaatcttacagcagctactgaccatgccgtcgggcgtggcagcggtaactgctgcgcgtgggcgcggtaaatcggcgctggcagggcaactcatttctcgtattgcgggtagtgcgattatcactgcgcccgcaaaagcggcaacggatgtactggcacaatttgcgggcgagaagtttcgctttattgcgcctgatgccttgttagccagcgatgagcaagccgactggctggtggtcgatgaagccgcagccatacctgcgccgttgttgcatcaactggtatcgcgttttcctcgaacgttgttaaccactacggtgcagggctacgaaggtaccggacgtggttttttgctgaaattttgcgctcgctttccgcatttacaccgttttgaactacaacagccgatccgctgggcacagggatgcccgctggaaaaaatggttagtgaggcactggtttttaacgatgaaaacttcacccatacaccacaaggcaatatcgtcatttccgcatttgaacaaacgttatggcgaagcgagccagaaacgccgttaaaggtttatcagttattgtctggtgcgcactaccggacttcgccgctggatttacgccgcatgatggatgcaccagggcaacattttttacaggcggctggcgaaaacgagattgccggagcgctgtggctggtggatgaggggggattatctcaagaactcagtcaggcggtatgggcaggttttcgtcgcccgcggggtaatctggtggcccagtcgctggcggcgcacggcagcaatccactggcggcgacattgcgtggacggcgggtcagccggatagcagttcatccggcgcgtcagcgcgaaggcgttgggcaacagctcattgccagcgctttgcaatataggcctggcctcgactatctttcggtgagttttggttacaccggggagttatggcgtttctggcaacgctgcggttttgtgctggtgcgaatgggtaatcatcgtgaagccagcagcggttgctatacggcgatggcgctgttaccgatgagtgatgcgggtaaacagctggctgaacgtgagcattaccgtttacgtcgcgatgcgcaagctctcgcgcagtggaatggcgaaacactccctgttgatccactaaacaatgccgtcctttctgacgacgactggcttgaactggccggttttgctttcgctcatcgtccgctattaacatcgttaggttgcttattgcgtctgctacaaaccagtgaactggcattaccggcgctgcgtgggcgtttacagaaaaacgtcagcgacgcgcagttatgtaccacacttaaactttcaggccgcaagatgttactggtccgtcagcgggaagaggccgcacaggcgctgttcgcacttaatgatgttcgcactgagcgtctgcgcgatcgcataacgcaatggcaattttttcactga

SEQ ID NO: 73 - DNA, 714 bp, Escherichia coli
atgttacgtcctgtagaaaccccaacccgtgaaatcaaaaaactcgacggcctgtgggcattcagtctggatcgcgaaaactgtggaattgagcagcgttggtgggaaagcgcgttacaagaaagccgggcaattgctgtgccaggcagttttaacgatcagttcgccgatgcagatattcgtaattatgtgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcacagtttgtgtgaacaacgaactgaactggcagactatcccgccgggaatggtgattaccgacgaaaacggcaagaaaaagcagtcttacttccatgatttctttaactacgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggggcaccagcgggactttgcaagtggtga

SEQ ID NO: 74 - DNA, 1113 bp, Escherichia coli
ttgcaagtggtgaatccgcacctctggcaaccgggtgaaggttatctctatgaactgtacgtcacagccaaaagccggacagagtgtgatatctacccgctgcgcgtcggcatccggtcagtggcagtgaagggcgaacagttcctgatcaaccacaaaccgttctactttactggctttggccgtcatgaagatgcggatttgcgcggcaaaggattcgataacgtgctgatggtgcacgatcacgcattaatggactggattggggccaactcctaccgtacctcgcattacccttacgctgaagagatgctcgactgggcagatgaacatggcatcgtggtgattgatgaaactgcagctgtcggctttaacctctctttaggcattggtttcgaagcgggcaacaagccgaaagaactgtacagcgaagaggcagtcaacggggaaactcagcaggcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgatccgacgcgtccgatcacctgcgtcaatgtaatgttctgcgacgctcacaccgataccatcagcgatctctttgatgtgctgtgcctgaaccgttattacggttggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaggggatcttcacccgcgaccgcaaaccgaagtcggcggcttttctgctgcaaaaacgctggactggcatgaacttcggtgaaaaaccgcagcagggaggcaaacaatga

SEQ ID NO: 75 - DNA, 1674 bp, Escherichia coli
atgcgcactttgttcgatggaaacaccgtgatgttgaagcgcctactaaaaagaccctctttgaatttactcgcctggctattgttggccgctttttatatctctatctgcctgaatattgccttttttaaacaggtgttgcaggcgctgccgctggactcgctgcataacgtactggttttcttgtcgatgccggtcgtagccttcagcgtgattaatattgtcctgacactaagctctttcttatggcttaatcgaccgctggcctgcctgtttattctggttggcgcggctgcacaatatttcataatgacttacggcatcgtcatcgaccgctcgatgattgccaatattattgataccactccggcagaaagctatgcgctgatgacaccgcaaatgttattaacgctgggattcagcggcgtgcttgctgcgctgattgcctgctggattaaaatcaaacctgccacctcgcgtctgcgcagtgttcttttccgtggagccaatattctggtttctgtactgctgattttgctggtcgccgcactattttataaagactacgcctcgttgttccgcaacaacaaagagctggtgaagtccttaagcccctctaacagcattgttgccagctggtcatggtactcccatcagcgactggcaaatctgccgctggtgcgaattggtgaagacgcgcaccgcaacccattaatgcagaacgaaaaacgtaaaaatttgaccatcctgattgtcggcgaaacctcgcgggcggagaacttctccctcaacggctacccgcgtgaaactaacccgcggttggcgaaagataacgtggtctatttccctaataccgcatcttgcggcacggcaacggcagtttcagtaccgtgcatgttctcggatatgccgcgagagcactacaaagaagagctggcacagcaccaggaaggcgtgctggatatcattcagcgagcgggcatcaacgtgctgtggaatgacaacgatggcggctgtaaaggtgcctgcgaccgcgtgcctcaccagaacgtcaccgcgctgaatctgcctgatcagtgcatcaacggcgaatgctatgacgaagtgctgttccacgggctggaagagtacatcaataacctgcagggtgatggcgtgattgtcttacacaccatcggcagccaccgtccgacctattacaaccgctatccgccgcagttcaggaaatttaccccaacctgcgacaccaatgagatccagacctgtaccaaagagcaactggtgaacacttacgacaacacgctggtttacgtcgactatattgttgataaagcgattaatctgctgaaagaacatcaggataaatttaccaccagcctggtttatctttctgaccacggtgaatcgttaggtgaaaatggcatctatctgcacggtctgccttatgccatcgccccggatagccaaaaacaggtgccgatgctgctgtggctgtcggaggattatcaaaaacggtatcaggttgaccagaactgcctgcaaaaacaggcgcaaacgcaacactattcacaagacaatttattctccacgctattgggattaactggcgttgagacgaagtattaccaggctgcggatgatattctgcaaacttgcaggagagtgagtgaatga

SEQ ID NO: 76 - DNA, 1344 bp, Escherichia coli
atgagaccaatatccaacctcaatacgagcgcagcaacctatattccaccgcaacaactcccgagtagttgtctggaaactttgacgcttctggtgcagatgaataattatatcaaaagggatgcggactacagcacgggcatggctgtagctccactggttcctgaagaggttcacttgctggctgctgcgatgacaaccgaactgcatcgacatcagttccaaccggtggtaagtgcaaatgatctgcaacttcctgaaccgttcaccttcgatgttgccggtttcaccatcaccttcacgaagacgcaagagcatgacaacacgggcaatctcatgaaaattgcagtcagcaagagcggtgtgtgcaccagtacaaatattactcttgagctgtttcactccattgtcacaaccctgatgtcacgcagtcaatatggaacctttgatctctggtccgtacggccaatactcacggatgaaagtcaaaacagggtgcatgaagcagctcgctactccccggcccagcaatacggaagagaagaaacacactttaccaacaatggcatgcgagaatttggaaatatgtctccgctggcgtggcgtaacgatgtggaattacagcaatcgtgtaatacaagcaacatcccaccaaatgctgcaaatgacgacataaacaacaccaggcagaccatcgaggaccagccagaggcggacgagccacagcaatatgttaaattgactgtagacgacatgcgaaaatgggcggctatggaccagcaggcgcgaaacgctctacagggagtttcaggatggtgtacgcgcaatcattttaatatcaaaaatgccagaaattacctgaccgatcacggcctgaactacgcgggacaggtaaaggtaaacaggccgcatgaatatgccaaattcacccttgaacatatccgccagtgggcagccttaaacaagcatgtgcgaaaaccggttggttatttagaaaagtggtgtaaggaaagaaacctcgcgccaactactgccaggaattacctgaagaacgacgggcttaccgccctgggtgagttgaagctgacagggccgcagaaatgggtcacgttcaccttcggagacatcgtacaatgggcgaatatgacccaggaggagcgtaacagcgcagggggagccgaaaagtggagtaaaaaacgcggcttccagtggtctactgccagatcctacctgaaatcgagcggagtgaccaagcaaggtgcgagaaaactggcgtggttaaaaaacagcgggaatatgtcgaatccattctatctcgcccaaccaaccagccgcaggacataa

SEQ ID NO: 77 - DNA, 1374 bp, Escherichia coli
atgcgcattcatattttaggaatttgtggcacgtttatgggcggtctggcgatgctggcgcgccagttaggccatgaagtaacgggttcggacgccaatgtgtatccgccgatgagcaccttacttgagaagcaaggcattgagctgattcagggttacgatgccagccagctcgacccgcagccggatctggtgattattggcaacgccatgacccgtggaaatccgtgtgtggaagcggtactggaaaaaaacatcccttatatgtcaggtccacagtggctgcacgattttgtgctgcgcgaccactgggtgctggccgttgccggtacacacggcaaaaccaccaccgcgggaatggcgacttggattctggaacagtgtggttacaaaccgggctttgtaatcggcggtgtgccggggaactttgaggtttcggcgcgtctgggcgaaagcgacttctttgttatcgaagcggatgagtacgactgcgccttcttcgacaaacgctctaaatttgtccattactgcccgcgtacgctgatcctcaacaaccttgagttcgatcacgccgatatctttgacgacctgaaagcgatccagaaacagttccaccatctggtgcgtatcgttccggggcagggtcgtattatctggccggaaaatgacatcaacctgaaacagaccatggcgatgggctgctggagcgagcaggagctggtgggtgagcagggtcactggcaggcgaaaaagctgaccaccgatgcttccgaatgggaagttttgctggatggcgaaaaagtgggcgaagtgaaatggtcgctggtaggcgaacataatatgcacaatggcctgatggcgattgcagcggctcgccatgttggtgtagcgccggcagatgccgctaacgcgctgggttcgtttattaacgctcgccgccgtctggagttgcgtggtgaagcgaatggcgtcacggtatatgacgattttgcccatcatccgacggcgattctggcaacgctggcggcgctgcgtggcaaagttggtggtacggcgcgcattattgctgtgctggagccgcgctcgaataccatgaaaatggggatctgcaaagacgatctggcaccttcattaggtcgtgccgatgaagtcttcctgctgcaaccggcgcatattccgtggcaggtggcagaagtggcagaagcctgcgttcagcctgcacactggagtggcgatgtggatacgctggcagatatggtggtgaaaaccgctcagcctggcgaccatattctggtgatgagcaacggcggttttggtgggatccatcagaaactactggatggtctggcgaagaaggcggaagctgcgcagtaa

SEQ ID NO: 78 - DNA, 1173 bp, Escherichia coli
gtggtacggggaaggaaaatcatgtcacttgtcaccgatctacccgccattttcgatcagttctctgaagctcgccagaaaggctttctcaccgtcatggatctcaaggtgcgcggcattccgctggttggcacttactgcacctttatgccgcaagagatcccgatggcagccggtgcggttgtggtttcgctctgttccacctctgatgaaaccattgaagaagcggagaaagatctgccgcgcaacctctgcccgctgattaaaagtagctacggcttcggcaaaaccgataaatgcccctacttctacttttcggatctggtggtcggtgaaaccacctgcgacggcaaaaagaaaatgtatgaatacatggcggagtttaagcccgttcatgtgatgcagctgccaaacagtgttaaagacgatgcctcgcgtgcgttatggaaagccgagatgctgcgcttacaaaaagcggtggaagaacgttttgggcacgaaattagcgaagatgctctgcgcgatgccattgcgctgaaaaaccgcgaacgtcgcgcactggccaatttttatcatcttgggcagttcaatcctccggcgcttagcggcagcgacattctgaaagtggtttacggcgcaaccttccggttcgataaagaggcgttgatcaatgaactggacgcgatgaccgcccgcattcgtcagcagtgggaagaaggccagcgactggacccgcgtccgcgcattttaatcaccggctgcccgattggcggcgcagcagagaaagtggtgcgcgcgattgaagagaatggcggctgggttgtcggttatgaaaactgcaccggggcgaaagcgaccgagcaatgcgtggcagaaacgggcgatgtctacgacgcgctggcggataaatatctggcgattggctgctcctgtgtttcgccgaacgatcagcgcctgaaaatgctcagccagatggtggaagaatatcaggtcgatggcgtagttgatgtgattttgcaggcgtgccatacctacgcggtggaatcgctggcaattaaacgtcatgtgcgtcagcagcacaacattccttatatcgctattgaaacagactactccacctcggatgttgggcagctcagtacccgtgtcgcggcctttattgagatgctgtaa

SEQ ID NO: 79 - DNA, 648 bp, Escherichia coli
atggcacgaaaaaccaaacaagaagcgcaagaaacgcgccaacacatcctcgatgtggctctacgtcttttctcacagcagggggtatcatccacctcgctgggtgagattgcaaaagcagctggcgttacgcgcggtgcaatctactggcattttaaagacaagtcggatttgttcagtgagatctgggaactatcagaatccaatattggtgaactagagcttgagtatcaggcaaaattccctagcgatccactctcagtattaagagagatattaattcatgttcttgaatccacggtgacagaagaacggcgtcgattattgatggagattatattccacaaatgcgaatttgtcggagaaatggctgttgtgcaacaggcacaacgtaatctctgtttggaaagttatgaccgtatagaacaaacgttaaaacattgtattgaagcgaaaatgttgcctgcggatttaatgacgcgtcgggcagcaattattatgcgcggctatatttccggcctgatggaaaactggctctttgccccgcaatcttttgatcttaaaaaagaagcccgcgattacgtcgccatcttactggagatgtatctcctgtgccccacgcttcgtaatcctgccactaacgaataa

SEQ ID NO: 80 - DNA, 1206 bp, Escherichia coli
ttgttatcgccgatccgtctttctccccttcccgccttgcgtcaggataacgatttcctttacgaccaaggagcgcccatggaacaacgccacatcaccggcaaaagccactggtatcatgaaacgcaatccagtactacggagtatgacgttctgcctctggtcccggaagccgcaaaggtcagcgatccctttctactcgacgtgatccttgaaaaagaaacgctggcccccttcctttcatggctggaccctgcgcgtgttcttgcagtggagttgttccctgaccagcttaccgtgacccgttcacagaccttcaccgcttatgaacgcttgtcgacggccctgacggttgctcaggtttgcggcgtccagcggttatgtaactactattcggcgcgacttacgccgctccccgggcctgattccaccagggaaagtaatcatcggttggcacaaatcacgcaatatgcccgccaactggctagctcgccttctattatcgacaaccgatcgcgccagcatctgaatgacgtcggtcttactgcctgggactgtgtgatcattaaccaaatcattggttttattggctttcaggcgcggacaattgcgacatttcaggcttatctcgggcatccggtacgctggttacccgggctggagatacaaaactacgccgacgcgtcactgtttgctgatgaatcattacgctggcgaagcagctatgaggtggaaaaactacctgaagaacacacaaaaagttcaactgcagaactttgccaactggccgaaatactctctctccaccctatttcactttcccttctcgaaaggttgttaaacagcacacgggttaatacacagccggataatcagcttgcggcgttgttatgcgcgcggataaatggcagtcctgcttgttttgccgcctgtatggattcatcaaatgaatataaaaaaatcagcccccttctgcgcaagggcgaaaatgaaattaaccaatgggctgaccgtcattctgttgagcgcgctaccgttcaggcgatacaatggctgacccgagcacccgatcgctttagcgccgcccagttcagccctttactcgaacacgaaaaatcatcaacgcagattattaatctgctggtatggagcgggctgtgtggctggataaatcgcttaaaaatcgcgttgggtgagacatattaa

SEQ ID NO: 81 - DNA, 2268 bp, Escherichia coli
atgaccaggccagtaacgttatcagaaccccatttcagccagcataccctgaacaagtatgcatcgctgatggcgcaggggaacggctatcttgggcttcgcgccagccatgaagaagattacacgcgccagacgcgagggatgtatctggcggggctgtatcatcgggcgggaaaaggtgaaatcaacgaactggtgaacctgcctgatatcttggggatggagattgccataaatggtgaggttttctcgttatcccacgaagcctggcagcgtgagcttgactttgccagtggcgaattacgccgcaacgttgtctggcgtaccagcaacggcgcaggttacaccatcgccagccgtcgctttgtttcggcagaccaactgccgctcattgcgctggaaatcactattacgccactggacgccgacgcgtcagtgctgatttcaacaggtatcgacgccacgcaaaccaaccacggtcgccaacatctcgacgaaacccaggtgcgggtgtttggtcagcatctgatgcaggggatctacaccacccaggatggacgcagtgatgtggccatcagctgttgctgtaaggtgagcggtgatgtgcagcaatgctataccgccaaagagcgccgtttgctgcaacataccagtgcgcagcttcatgcaggcgagacagtgacgttgcaaaaactggtgtggatcgactggcgggatgacaggcaagccgttttagacgagtggggcagcgcgtcgcttcgccagcttgaaatatgcgcgcagcagagttacgaccaacttcttgcagcatcaacagaaaactggcgtcaatggtggcagaaacgtcgtatcacggtaaatggcggcgatgcgcacgatcagcaagcgttagattatgcgctttatcatctgcgcatcatgacgccggctcacgacgagcgcagcagtattgcggcaaaaggcttaaccggcgaaggctacaaaggccacgttttctgggatacagaagtatttttgctgccgttccatctgtttagcgatccgacggttgcccgaagtttactgcgttatcgctggcacaacttgccaggcgcgcaggagaaagcacggcgcagcggctggcagggcgcgctatttccgtgggaaagcgcgcgcagcggcgaagaagagacgccagaatttgccgccattaacattcgtaccgggctgcggcaaaaagtggcctcggcgcaggcggaacatcatctggtggccgatatcgcctgggcggttattcaatactggcagaccacgggggatgaaagtttcattgctcatgaaggcatggcgctacttctggaaactgcaaagttctggattagccgcgcggtgagggttaacgaccgtctggaaattcatgatgttattgggccagacgaatataccgaacatgtcaataataacgccttcaccagctatatggcccgctacaacgttcaacaggcgctgaatattgcccgccagttcggctgtagcgacgatgcgtttatccatcgcgccgaaatgttcctcaaagagctatggatgccagaaacgcagcccgatggcgttttgccgcaggatgattcgtttatggctaagccggcgattaatctggctaaatacaaagcggcggcggggaagcaaaccattctgctggattattcacgcgcagaagtgaacgagatgcagatcctcaaacaagctgatgtggtgatgctcaattacatgctgccggagcagttctcagcggtatcgtgtcttgccaatctgcaattttatgaaccgcgcactattcacgactcgtcattaagtaaagcaatccacggcattgttgccgcacgctgtggcctgctgacccaaagttatcagttctggcgcgaggggactgaaatcgatcttggtgctgatccgcatagttgtgatgatggtatccacgctgccgcaactggcgctatctggctgggggcgattcagggttttgccggggtgagcgtgcgtgacggtgaattacatctcaatccggcgttaccggagcagtggcaacagttgtctttccctctgttctggcagggctgcgaattacaggtcacgctcgacgcgcagcgtattgcgattcgaacttctgcgcccgtttcactgcgtttgaacgggcagcttatatccgtggctgaagaatctgttttctgtttgggtgattttattttgcccttcaatgggaccgctaccacgcatcaggaggatgaatga

SEQ ID NO: 82 - DNA, 1395 bp, Escherichia coli
atgacaaatctaaaaaagcgcgagcgagcgaaaaccaatgcatcgttaatctctatggtgcaacgcttttcagatatcaccatcatgtttgccggactatggctggtttgcgaagtgagcggactgtcattcctctacatgcacctgttggtggcgctgattacgctggtggtgttccagatgctgggcggcatcaccgatttttatcgctcatggcgcggtgttcgggcagcgacagaatttgccctgttgctacaaaactggaccttaagcgtgattttcagcgccggactggtggcgttcaacaatgatttcgacacgcaactgaaaatctggctggcgtggtatgggctgaccagtatcggactggtggtttgccgttcatgtattcgcattggggcgggctggctgcgtaatcatggctataacaagcgcatggtcgcggtggcgggggatttagccgccgggcaaatgctgatggagagcttccgtaaccagccgtggttagggtttgaagtggtgggcgtttaccacgacccgaaactgggcggcgtttctaacgactgggcgggtaacctgcaacagctggtcgaggatgctaaagcaggcaagattcataacgtctatatcgcgatgcaaatgtgcgatggcgcgcgagtgaaaaaactggtccatcaactggcggacaccacctgttcggtgctgctgatccccgatgtctttaccttcaacattctccattcacgtcttgaagagatgaacggcgttccggtggtgccgctttacgacacgccgctttccggggttaaccgcctgctcaaacgtgcggaagacattgtgctggcgacgcttatcctgctgctgatctccccggtgctgtgctgcattgcgctggcggtgaaactcagttcacctgggccggttattttccgccagactcgctacggcatggatggcaagccgatcaaagtgtggaagttccgttccatgaaagtgatggagaacgacaaagtggtgacccaggcgacgcagaacgatccgcgcgtcaccaaagtggggaactttctgcgccgcacctcgctggatgaattgccgcagtttatcaatgtgctgaccggggggatgtcgattgtcggtccacgtccgcacgcggtggcgcataacgaacagtatcgacagctcattgaaggctacatgctgcgtcataaggtgaaaccgggcattaccggctgggcgcagattaacggctggcgcggcgaaaccgacacgctggagaaaatggaaaaacgcgtcgagttcgaccttgagtacatccgcgaatggagcgtctggttcgatatcaaaatcgttttcctgacggtattcaaaggattcgttaacaaagcggcatattga

SEQ ID NO: 83 - DNA, 31 bp, Artificial Sequence
  primer for escherichia coli
cgaaggcagcacttcactgatattgccttcg

SEQ ID NO: 84 - DNA, 31 bp, Artificial Sequence
  primer for escherichia coli
agaaggcagcacttcactgatattgccttct

SEQ ID NO: 85 - DNA, 16 bp, Artificial Sequence
  primer for escherichia coli
gcaaaccgccagcggc

SEQ ID NO: 86 - DNA, 27 bp, Artificial Sequence
  primer for escherichia coli
acggcttggcagtttttccaaagccgt

SEQ ID NO: 87 - DNA, 23 bp, Artificial Sequence
  primer for escherichia coli
tcagcttggtgttaagacgttcc

SEQ ID NO: 88 - DNA, 27 bp, Artificial Sequence
  primer for escherichia coli
gcggcttggcagtttttccaaagccgc

SEQ ID NO: 89 - DNA, 37 bp, Artificial Sequence
  primer for escherichia coli
gagcaattgtcagtcgacgaactcataacaattgctc

SEQ ID NO: 90 - DNA, 37 bp, Artificial Sequence
  primer for escherichia coli
aagcaattgtcagtcgacgaactcataacaattgctt

SEQ ID NO: 91 - DNA, 26 bp, Artificial Sequence
  primer for escherichia coli
tgaccgaaaccattgagaataatttt

SEQ ID NO: 92 - DNA, 29 bp, Artificial Sequence
  primer for escherichia coli
gttgcggcagctataacggtatccgcaac

SEQ ID NO: 93 - DNA, 22 bp, Artificial Sequence
  primer for escherichia coli
cataaaatcggtaccagcaacg

SEQ ID NO: 94 - DNA, 29 bp, Artificial Sequence
  primer for escherichia coli
attgcggcagctataacggtatccgcaat

SEQ ID NO: 95 - DNA, 31 bp, Artificial Sequence
  primer for escherichia coli
tagggaactgagtatcaggcaaagttcccta

SEQ ID NO: 96 - DNA, 21 bp, Artificial Sequence
  primer for escherichia coli
gtcaccgtggattcaagaaca

SEQ ID NO: 97 - DNA, 31 bp, Artificial Sequence
  primer for escherichia coli
cagggaactgagtatcaggcaaagttccctg

SEQ ID NO: 98 - DNA, 33 bp, Artificial Sequence
  primer for escherichia coli
aaatgcctcagcggtgtaaaagaaaaggcattt

SEQ ID NO: 99 - DNA, 25 bp, Artificial Sequence
  primer for escherichia coli
tattttcgcttttgggttcactaac

SEQ ID NO: 100 - DNA, 33 bp, Artificial Sequence
  primer for escherichia coli
gaatgcctcagcggtgtaaaagaaaaggcattc

SEQ ID NO: 101 - DNA, 25 bp, Artificial Sequence
  primer for escherichia coli
acccgtggttgcctgtgaaacgggt

SEQ ID NO: 102 - DNA, 17 bp, Artificial Sequence
  primer for escherichia coli
tcgcaatggcaggatca

SEQ ID NO: 103 - DNA, 25 bp, Artificial Sequence
  primer for escherichia coli
ccccgtggttgcctgtgaaacgggg

SEQ ID NO: 104 - DNA, 28 bp, Artificial Sequence
  primer for escherichia coli
gggaccagcttgaactggccctggtccc

SEQ ID NO: 105 - DNA, 25 bp, Artificial Sequence
  primer for escherichia coli
ggatctcaatactcaaatcaccgtg

SEQ ID NO: 106 - DNA, 28 bp, Artificial Sequence
  primer for escherichia coli
aggaccagcttgaactggccctggtcct

SEQ ID NO: 107 - DNA, 26 bp, Artificial Sequence
  primer for escherichia coli
agcgcttaccaggctgaaaaagcgct

SEQ ID NO: 108 - DNA, 20 bp, Artificial Sequence
  primer for escherichia coli
atgccgtcctgtaaaccaga

SEQ ID NO: 109 - DNA, 26 bp, Artificial Sequence
  primer for escherichia coli
cgcgcttaccaggctgaaaaagcgcg

SEQ ID NO: 110 - DNA, 31 bp, Artificial Sequence
  primer for escherichia coli
atccggtgaagatgggctttaaaaaccggat

SEQ ID NO: 111 - DNA, 20 bp, Artificial Sequence
  primer for escherichia coli
cgaatgtgttctaccagcgg

SEQ ID NO: 112 - DNA, 31 bp, Artificial Sequence
  primer for escherichia coli
gtccggtgaagatgggctttaaaaaccggac

SEQ ID NO: 113 - DNA, 27 bp, Artificial Sequence
  primer for escherichia coli
gtccgtgtttcacctaatgccacggac

SEQ ID NO: 114 - DNA, 18 bp, Artificial Sequence
  primer for escherichia coli
gccgcagcatgttgtttg

SEQ ID NO: 115 - DNA, 27 bp, Artificial Sequence
  primer for escherichia coli
ctccgtgtttcacctaatgccacggag

SEQ ID NO: 116 - DNA, 30 bp, Artificial Sequence
  primer for escherichia coli
atcagctttggtacgcgcgataaagctgat

SEQ ID NO: 117 - DNA, 15 bp, Artificial Sequence
  primer for escherichia coli
gcagccaggcggtgc

SEQ ID NO: 118 - DNA, 30 bp, Artificial Sequence
  primer for escherichia coli
gtcagctttggtacgcgcgataaagctgac

SEQ ID NO: 119 - DNA, 29 bp, Artificial Sequence
  primer for escherichia coli
gtacgcttcagcagtttttcgaagcgtac

SEQ ID NO: 120 - DNA, 19 bp, Artificial Sequence
  primer for escherichia coli
ctccggcagaagatatggc

SEQ ID NO: 121 - DNA, 29 bp, Artificial Sequence
  primer for escherichia coli
ctacgcttcagcagtttttcgaagcgtag

SEQ ID NO: 122 - DNA, 27 bp, Artificial Sequence
  primer for escherichia coli
cagggcactttattgtcggctgccctg

SEQ ID NO: 123 - DNA, 25 bp, Artificial Sequence
  primer for escherichia coli
aagtcgagtagcatctggaaatctt

SEQ ID NO: 124 - DNA, 27 bp, Artificial Sequence
  primer for escherichia coli
tagggcactttattgtcggctgcccta

SEQ ID NO: 125 - DNA, 21 bp, Artificial Sequence
  primer for escherichia coli
tcgctgggaagatggcagcga

SEQ ID NO: 126 - DNA, 22 bp, Artificial Sequence
  primer for escherichia coli
cccacgaactgtagcgattatg

SEQ ID NO: 127 - DNA, 21 bp, Artificial Sequence
  primer for escherichia coli
ccgctgggaagatggcagcgg

SEQ ID NO: 128 - DNA, 27 bp, Artificial Sequence
  primer for escherichia coli
agcaacgttcgcccttttatcgttgct

SEQ ID NO: 129 - DNA, 15 bp, Artificial Sequence
  primer for escherichia coli
aatcgcgttccgccg

SEQ ID NO: 130 - DNA, 27 bp, Artificial Sequence
  primer for escherichia coli
ggcaacgttcgcccttttatcgttgcc

SEQ ID NO: 131 - DNA, 31 bp, Artificial Sequence
  primer for escherichia coli
tcagagcataacatgcaaacttgtgctctga

SEQ ID NO: 132 - DNA, 22 bp, Artificial Sequence
  primer for escherichia coli
caccgtctctctcctttcgatg

SEQ ID NO: 133 - DNA, 31 bp, Artificial Sequence
  primer for escherichia coli
ccagagcataacatgcaaacttgtgctctgg

SEQ ID NO: 134 - DNA, 32 bp, Artificial Sequence
  primer for escherichia coli
agatatccagcttatggcagcactggatatct

SEQ ID NO: 135 - DNA, 23 bp, Artificial Sequence
  primer for escherichia coli
ttcttaatttcttctgccaggga

SEQ ID NO: 136 - DNA, 32 bp, Artificial Sequence
  primer for escherichia coli
tgatatccagcttatggcagcactggatatca

SEQ ID NO: 137 - DNA, 33 bp, Artificial Sequence
  primer for escherichia coli
caacaaccactccaggtggtagcgtggttgttg

SEQ ID NO: 138 - DNA, 19 bp, Artificial Sequence
  primer for escherichia coli
tgactctgcaggcgcagaa

SEQ ID NO: 139 - DNA, 33 bp, Artificial Sequence
  primer for escherichia coli
taacaaccactccaggtggtagcgtggttgtta

SEQ ID NO: 140 - DNA, 28 bp, Artificial Sequence
  primer for escherichia coli
acgtaccaacgccaataacctggtacgt

SEQ ID NO: 141 - DNA, 19 bp, Artificial Sequence
  primer for escherichia coli
tggtcacttcacccgcatc

SEQ ID NO: 142 - DNA, 28 bp, Artificial Sequence
  primer for escherichia coli
gcgtaccaacgccaataacctggtacgc

SEQ ID NO: 143 - DNA, 29 bp, Artificial Sequence
  primer for escherichia coli
cacacagtcttactgcctgcgactgtgtg

SEQ ID NO: 144 - DNA, 29 bp, Artificial Sequence
  primer for escherichia coli
tacacagtcttactgcctgcgactgtgta

SEQ ID NO: 145 - DNA, 20 bp, Artificial Sequence
  primer for escherichia coli
gtaccggatgcccgagataa

SEQ ID NO: 146 - DNA, 28 bp, Artificial Sequence
  primer for escherichia coli
taccacgtcatcctcctgatacgtggta

SEQ ID NO: 147 - DNA, 28 bp, Artificial Sequence
  primer for escherichia coli
caccacgtcatcctcctgatacgtggtg

SEQ ID NO: 148 - DNA, 23 bp, Artificial Sequence
  primer for escherichia coli
tatccgtggctgaagaatctgtt

SEQ ID NO: 149 - DNA, 31 bp, Artificial Sequence
  primer for escherichia coli
aggtcattgtgtcctggtgcgtcaatgacct

SEQ ID NO: 150 - DNA, 19 bp, Artificial Sequence
  primer for escherichia coli
tgctgccaccggctaatgt

SEQ ID NO: 151 - DNA, 31 bp, Artificial Sequence
  primer for escherichia coli
gggtcattgtgtcctggtgcgtcaatgaccc

SEQ ID NO: 152 - DNA, 31 bp, Artificial Sequence
  primer for escherichia coli
cacaagacgcctagatatcccacgtcttgtg

SEQ ID NO: 153 - DNA, 15 bp, Artificial Sequence
  primer for escherichia coli
cgtgccgaccagcga

SEQ ID NO: 154 - DNA, 31 bp, Artificial Sequence
  primer for escherichia coli
tacaagacgcctagatatcccacgtcttgta

SEQ ID NO: 155 - DNA, 23 bp, Artificial Sequence
  primer for escherichia coli
ccgagcgttttccagtggctcgg

SEQ ID NO: 156 - DNA, 21 bp, Artificial Sequence
  primer for escherichia coli
gaatctgcaggccaaaatttc

SEQ ID NO: 157 - DNA, 23 bp, Artificial Sequence
  primer for escherichia coli
acgagcgttttccagtggctcgt

SEQ ID NO: 158 - DNA, 34 bp, Artificial Sequence
  primer for escherichia coli
ggagtttgttgtcgcttctacaccaacaaactcc

SEQ ID NO: 159 - DNA, 34 bp, Artificial Sequence
  primer for escherichia coli
agagtttgttgtcgcttctacaccaacaaactct

SEQ ID NO: 160 - DNA, 20 bp, Artificial Sequence
  primer for escherichia coli
cactgtatggcatcccgaca

SEQ ID NO: 161 - DNA, 34 bp, Artificial Sequence
  primer for escherichia coli
agtgtaactgcgcaactgccagaacagttacact

SEQ ID NO: 162 - DNA, 18 bp, Artificial Sequence
  primer for escherichia coli
ttcggagccccggttatt

SEQ ID NO: 163 - DNA, 34 bp, Artificial Sequence
  primer for escherichia coli
cgtgtaactgcgcaactgccagaacagttacacg

SEQ ID NO: 164 - DNA, 28 bp, Artificial Sequence
  primer for escherichia coli
cgtgaagcggatgcagaacggcttcacg

SEQ ID NO: 165 - DNA, 28 bp, Artificial Sequence
  primer for escherichia coli
tgtgaagcggatgcagaacggcttcaca

SEQ ID NO: 166 - DNA, 20 bp, Artificial Sequence
  primer for escherichia coli
ttcattctgccgctgaatgc

SEQ ID NO: 167 - DNA, 27 bp, Artificial Sequence
  primer for escherichia coli
gaccagacgggcgtctacggtctggtc

SEQ ID NO: 168 - DNA, 27 bp, Artificial Sequence
  primer for escherichia coli
taccagacgggcgtctacggtctggta

SEQ ID NO: 169 - DNA, 18 bp, Artificial Sequence
  primer for escherichia coli
ccagccaataccccaggt

SEQ ID NO: 170 - DNA, 28 bp, Artificial Sequence
  primer for escherichia coli
cccgtgaagttacctttaaggtcacggg

SEQ ID NO: 171 - DNA, 28 bp, Artificial Sequence
  primer for escherichia coli
accgtgaagttacctttaaggtcacggt

SEQ ID NO: 172 - DNA, 24 bp, Artificial Sequence
  primer for escherichia coli
gactatcttcgtatcgttgttgcc

SEQ ID NO: 173 - DNA, 25 bp, Artificial Sequence
  primer for escherichia coli
atcgcacgcgatgcaaaggtgcgat

SEQ ID NO: 174 - DNA, 25 bp, Artificial Sequence
  primer for escherichia coli
gtcgcacgcgatgcaaaggtgcgac

SEQ ID NO: 175 - DNA, 18 bp, Artificial Sequence
  primer for escherichia coli
cgaacaggtggtgtccgc

SEQ ID NO: 176 - DNA, 27 bp, Artificial Sequence
  primer for escherichia coli
tgcgatgttcaggttagtgccatcgca

SEQ ID NO: 177 - DNA, 23 bp, Artificial Sequence
  primer for escherichia coli
gccttcattggcactacacagat

SEQ ID NO: 178 - DNA, 27 bp, Artificial Sequence
  primer for escherichia coli
ggcgatgttcaggttagtgccatcgcc

SEQ ID NO: 179 - DNA, 23 bp, Artificial Sequence
  primer for escherichia coli
gccccagacccttgaaatggggc

SEQ ID NO: 180 - DNA, 18 bp, Artificial Sequence
  primer for escherichia coli
tctgcctgcgatttccct

SEQ ID NO: 181 - DNA, 23 bp, Artificial Sequence
  primer for escherichia coli
tccccagacccttgaaatgggga

SEQ ID NO: 182 - DNA, 25 bp, Artificial Sequence
  primer for escherichia coli
tgccaccaggatccccagagtggca

SEQ ID NO: 183 - DNA, 18 bp, Artificial Sequence
  primer for escherichia coli
gctcgactttgttcgcgg

SEQ ID NO: 184 - DNA, 25 bp, Artificial Sequence
  primer for escherichia coli
cgccaccaggatccccagagtggcg

SEQ ID NO: 185 - DNA, 27 bp, Artificial Sequence
  primer for escherichia coli
ttgcgtcgttccagcttatggacgcaa

SEQ ID NO: 186 - DNA, 20 bp, Artificial Sequence
  primer for escherichia coli
tgccgctacatcaccgttca

SEQ ID NO: 187 - DNA, 27 bp, Artificial Sequence
  primer for escherichia coli
ctgcgtcgttccagcttatggacgcag

SEQ ID NO: 188 - DNA, 23 bp, Artificial Sequence
  primer for escherichia coli
ccctgccagtccatggtgcaggg

SEQ ID NO: 189 - DNA, 20 bp, Artificial Sequence
  primer for escherichia coli
ccgagaacttacggtagcca

SEQ ID NO: 190 - DNA, 23 bp, Artificial Sequence
  primer for escherichia coli
gcctgccagtccatggtgcaggc

SEQ ID NO: 191 - DNA, 31 bp, Artificial Sequence
  primer for escherichia coli
tagttcaacgcatttacaccgtgttgaacta

SEQ ID NO: 192 - DNA, 31 bp, Artificial Sequence
  primer for escherichia coli
cagttcaacgcatttacaccgtgttgaactg

SEQ ID NO: 193 - DNA, 19 bp, Artificial Sequence
  primer for escherichia coli
aaccattttttccagcggg

SEQ ID NO: 194 - DNA, 21 bp, Artificial Sequence
  primer for escherichia coli
ccaccggcgagctagcggtgg

SEQ ID NO: 195 - DNA, 23 bp, Artificial Sequence
  primer for escherichia coli
gtgcgcaaaatgtatgaattacg

SEQ ID NO: 196 - DNA, 21 bp, Artificial Sequence
  primer for escherichia coli
tcaccggcgagctagcggtga

SEQ ID NO: 197 - DNA, 30 bp, Artificial Sequence
  primer for escherichia coli
tccatcatagataaagaccgctatgatgga

SEQ ID NO: 198 - DNA, 30 bp, Artificial Sequence
  primer for escherichia coli
gccatcatagataaagaccgctatgatggc

SEQ ID NO: 199 - DNA, 18 bp, Artificial Sequence
  primer for escherichia coli
tgatcctgccaggcgact

SEQ ID NO: 200 - DNA, 30 bp, Artificial Sequence
  primer for escherichia coli
tagtgctttgccgcagaattaaaagcacta

SEQ ID NO: 201 - DNA, 30 bp, Artificial Sequence
  primer for escherichia coli
cagtgctttgccgcagaattaaaagcactg

SEQ ID NO: 202 - DNA, 20 bp, Artificial Sequence
  primer for escherichia coli
ttgtcgcggaatacggaaat

SEQ ID NO: 203 - DNA, 21 bp, Artificial Sequence
  primer for escherichia coli
tgcccaccctacgactgggca

SEQ ID NO: 204 - DNA, 22 bp, Artificial Sequence
  primer for escherichia coli
tccctctcgaatcaacaacatg

SEQ ID NO: 205 - DNA, 21 bp, Artificial Sequence
  primer for escherichia coli
cgcccaccctacgactgggcg

SEQ ID NO: 206 - DNA, 26 bp, Artificial Sequence
  primer for escherichia coli
gtgcgggccgggtatttacaccgcac

SEQ ID NO: 207 - DNA, 20 bp, Artificial Sequence
  primer for escherichia coli
gattcttttgatcggtcgcg

SEQ ID NO: 208 - DNA, 26 bp, Artificial Sequence
  primer for escherichia coli
atgcgggccgggtatttacaccgcat

SEQ ID NO: 209 - DNA, 28 bp, Artificial Sequence
  primer for escherichia coli
tgctgcactggaaggtgtcgctgcagca

SEQ ID NO: 210 - DNA, 28 bp, Artificial Sequence
  primer for escherichia coli
cgctgcactggaaggtgtcgctgcagcg

SEQ ID NO: 211 - DNA, 16 bp, Artificial Sequence
  primer for escherichia coli
gcaccgagcgcgatga

SEQ ID NO: 212 - DNA, 30 bp, Artificial Sequence
  primer for escherichia coli
agtgcacattacgactaagacgtgtgcact

SEQ ID NO: 213 - DNA, 18 bp, Artificial Sequence
  primer for escherichia coli
ggacaggcgaccatgcag

SEQ ID NO: 214 - DNA, 30 bp, Artificial Sequence
  primer for escherichia coli
ggtgcacattacgactaagacgtgtgcacc

SEQ ID NO: 215 - DNA, 25 bp, Artificial Sequence
  primer for escherichia coli
tgcgtaacgaacgacgggttacgca

SEQ ID NO: 216 - DNA, 23 bp, Artificial Sequence
  primer for escherichia coli
ggcaataacacactgacgtttgg

SEQ ID NO: 217 - DNA, 25 bp, Artificial Sequence
  primer for escherichia coli
ggcgtaacgaacgacgggttacgcc

SEQ ID NO: 218 - DNA, 26 bp, Artificial Sequence
  primer for escherichia coli
tgcgatgagcttttggtaccatcgca

SEQ ID NO: 219 - DNA, 26 bp, Artificial Sequence
  primer for escherichia coli
ggcgatgagcttttggtaccatcgcc

SEQ ID NO: 220 - DNA, 19 bp, Artificial Sequence
  primer for escherichia coli
agtaaccaggttcccgcca

SEQ ID NO: 221 - DNA, 28 bp, Artificial Sequence
  primer for escherichia coli
caggctgacgcgaagttccatcagcctg

SEQ ID NO: 222 - DNA, 22 bp, Artificial Sequence
  primer for escherichia coli
ctgacaatcgtaccgataaccg

SEQ ID NO: 223 - DNA, 28 bp, Artificial Sequence
  primer for escherichia coli
taggctgacgcgaagttccatcagccta

SEQ ID NO: 224 - DNA, 29 bp, Artificial Sequence
  primer for escherichia coli
cgtcacacatccatactcatggtgtgacg

SEQ ID NO: 225 - DNA, 20 bp, Artificial Sequence
  primer for escherichia coli
tcagtagcaatccccggata

SEQ ID NO: 226 - DNA, 29 bp, Artificial Sequence
  primer for escherichia coli
tgtcacacatccatactcatggtgtgaca

SEQ ID NO: 227 - DNA, 31 bp, Artificial Sequence
  primer for escherichia coli
tggcttaatctgtactgcgttgattaagcca

SEQ ID NO: 228 - DNA, 20 bp, Artificial Sequence
  primer for escherichia coli
ggcaccgttgtgctgcttat

SEQ ID NO: 229 - DNA, 31 bp, Artificial Sequence
  primer for escherichia coli
cggcttaatctgtactgcgttgattaagccg

SEQ ID NO: 230 - DNA, 24 bp, Artificial Sequence
  primer for escherichia coli
gctccacagtccaggaagtggagc

SEQ ID NO: 231 - DNA, 24 bp, Artificial Sequence
  primer for escherichia coli
ctatttgtgcatggtattcaatgg

SEQ ID NO: 232 - DNA, 24 bp, Artificial Sequence
  primer for escherichia coli
actccacagtccaggaagtggagt

SEQ ID NO: 233 - DNA, 26 bp, Artificial Sequence
  primer for escherichia coli
aaaccctgtgggtcagctcagggttt

SEQ ID NO: 234 - DNA, 23 bp, Artificial Sequence
  primer for escherichia coli
gtgttcttcttctacccagcctg

SEQ ID NO: 235 - DNA, 26 bp, Artificial Sequence
  primer for escherichia coli
caaccctgtgggtcagctcagggttg

SEQ ID NO: 236 - DNA, 26 bp, Artificial Sequence
  primer for escherichia coli
ccaacggaaaatcagcagaccgttgg

SEQ ID NO: 237 - DNA, 22 bp, Artificial Sequence
  primer for escherichia coli
tttataagaaagctgcgcatcg

SEQ ID NO: 238 - DNA, 26 bp, Artificial Sequence
  primer for escherichia coli
tcaacggaaaatcagcagaccgttga

SEQ ID NO: 239 - DNA, 28 bp, Artificial Sequence
  primer for escherichia coli
tacaaggggcacagcgaatacccttgta

SEQ ID NO: 240 - DNA, 28 bp, Artificial Sequence
  primer for escherichia coli
gacaaggggcacagcgaatacccttgtc

SEQ ID NO: 241 - DNA, 22 bp, Artificial Sequence
  primer for escherichia coli
caggatgctggcccagtaactt

SEQ ID NO: 242 - DNA, 27 bp, Artificial Sequence
  primer for escherichia coli
cactcgacggctttagagggtcgagtg

SEQ ID NO: 243 - DNA, 20 bp, Artificial Sequence
  primer for escherichia coli
ccattctctgtggcgtcaat

SEQ ID NO: 244 - DNA, 27 bp, Artificial Sequence
  primer for escherichia coli
aactcgacggctttagagggtcgagtt

SEQ ID NO: 245 - DNA, 24 bp, Artificial Sequence
  primer for escherichia coli
gcgcctctgagctattgaaggcgc

SEQ ID NO: 246 - DNA, 27 bp, Artificial Sequence
  primer for escherichia coli
agaaaaataatcaaatgaaagcaaacg

SEQ ID NO: 247 - DNA, 24 bp, Artificial Sequence
  primer for escherichia coli
acgcctctgagctattgaaggcgt

SEQ ID NO: 248 - DNA, 31 bp, Artificial Sequence
  primer for escherichia coli
tccatatccactttcaccgaatggatatgga

SEQ ID NO: 249 - DNA, 25 bp, Artificial Sequence
  primer for escherichia coli
aatagctgaacagtaaccgcgttag

SEQ ID NO: 250 - DNA, 31 bp, Artificial Sequence
  primer for escherichia coli
cccatatccactttcaccgaatggatatggg

SEQ ID NO: 251 - DNA, 27 bp, Artificial Sequence
  primer for escherichia coli
gtgcctgttccaccctatgacaggcac

SEQ ID NO: 252 - DNA, 27 bp, Artificial Sequence
  primer for escherichia coli
ttgcctgttccaccctatgacaggcaa

SEQ ID NO: 253 - DNA, 16 bp, Artificial Sequence
  primer for escherichia coli
tgccgccacccaggta

SEQ ID NO: 254 - DNA, 35 bp, Artificial Sequence
  primer for escherichia coli
tcagaagctttatagtgtaaggcaagagcttctga

SEQ ID NO: 255 - DNA, 35 bp, Artificial Sequence
  primer for escherichia coli
ccagaagctttatagtgtaaggcaagagcttctgg

SEQ ID NO: 256 - DNA, 32 bp, Artificial Sequence
  primer for escherichia coli
tataagagagaatctctccatcatttttatat

SEQ ID NO: 257 - DNA, 23 bp, Artificial Sequence
  primer for escherichia coli
gccttcgcagccgcatcgaaggc

SEQ ID NO: 258 - DNA, 27 bp, Artificial Sequence
  primer for escherichia coli
cgactgaatgttaaataaatattgccc

SEQ ID NO: 259 - DNA, 23 bp, Artificial Sequence
  primer for escherichia coli
accttcgcagccgcatcgaaggt

SEQ ID NO: 260 - DNA, 25 bp, Artificial Sequence
  primer for escherichia coli
tcctggagctgctggaagtccagga

SEQ ID NO: 261 - DNA, 22 bp, Artificial Sequence
  primer for escherichia coli
cgctttatcaccaaagaaggcc

SEQ ID NO: 262 - DNA, 25 bp, Artificial Sequence
  primer for escherichia coli
ccctggagctgctggaagtccaggg

SEQ ID NO: 263 - DNA, 27 bp, Artificial Sequence
  primer for escherichia coli
agactccaacccatcagcgtggagtct

SEQ ID NO: 264 - DNA, 27 bp, Artificial Sequence
  primer for escherichia coli
ggactccaacccatcagcgtggagtcc

SEQ ID NO: 265 - DNA, 16 bp, Artificial Sequence
  primer for escherichia coli
cccgctttggttccgg

SEQ ID NO: 266 - DNA, 25 bp, Artificial Sequence
  primer for escherichia coli
gggcgacttacaaaagcaatcgccc

SEQ ID NO: 267 - DNA, 24 bp, Artificial Sequence
  primer for escherichia coli
gaagatgtctatccgattctgtcg

SEQ ID NO: 268 - DNA, 25 bp, Artificial Sequence
  primer for escherichia coli
aggcgacttacaaaagcaatcgcct

SEQ ID NO: 269 - DNA, 28 bp, Artificial Sequence
  primer for escherichia coli
aaccacgtgggtactggtcgtcgtggtt

SEQ ID NO: 270 - DNA, 15 bp, Artificial Sequence
  primer for escherichia coli
gtgtcgcgctcgcgg

SEQ ID NO: 271 - DNA, 28 bp, Artificial Sequence
  primer for escherichia coli
gaccacgtgggtactggtcgtcgtggtc

SEQ ID NO: 272 - DNA, 33 bp, Artificial Sequence
  primer for escherichia coli
tagtccttggtgttaaatctcgatcaaggacta

SEQ ID NO: 273 - DNA, 33 bp, Artificial Sequence
  primer for escherichia coli
cagtccttggtgttaaatctcgatcaaggactg

SEQ ID NO: 274 - DNA, 21 bp, Artificial Sequence
  primer for escherichia coli
cttgcaccaccgattcaaaat

SEQ ID NO: 275 - DNA, 24 bp, Artificial Sequence
  primer for escherichia coli
ggtggctcaccataggcagccacc

SEQ ID NO: 276 - DNA, 21 bp, Artificial Sequence
  primer for escherichia coli
gtaaatttcctgaactgcggc

SEQ ID NO: 277 - DNA, 24 bp, Artificial Sequence
  primer for escherichia coli
cgtggctcaccataggcagccacg

SEQ ID NO: 278 - DNA, 21 bp, Artificial Sequence
  primer for escherichia coli
cgggctcgctctccaagcccg

SEQ ID NO: 279 - DNA, 19 bp, Artificial Sequence
  primer for escherichia coli
gaaggtgtgcgaatgccaa

SEQ ID NO: 280 - DNA, 21 bp, Artificial Sequence
  primer for escherichia coli
tgggctcgctctccaagccca

SEQ ID NO: 281 - DNA, 28 bp, Artificial Sequence
  primer for escherichia coli
tgattgacggtatgacccgcgtcaatca

SEQ ID NO: 282 - DNA, 17 bp, Artificial Sequence
  primer for escherichia coli
ctggcacaggacggagc

SEQ ID NO: 283 - DNA, 28 bp, Artificial Sequence
  primer for escherichia coli
cgattgacggtatgacccgcgtcaatcg

SEQ ID NO: 284 - DNA, 31 bp, Artificial Sequence
  primer for escherichia coli
cgtcgtaacggcatcacctcgagttacgacg

SEQ ID NO: 285 - DNA, 31 bp, Artificial Sequence
  primer for escherichia coli
tgtcgtaacggcatcacctcgagttacgaca

SEQ ID NO: 286 - DNA, 26 bp, Artificial Sequence
  primer for escherichia coli
ctttagtgatgtggatgagtccatca

SEQ ID NO: 287 - DNA, 32 bp, Artificial Sequence
  primer for escherichia coli
acgtcactttcctcttagtacaacagtgacgt

SEQ ID NO: 288 - DNA, 32 bp, Artificial Sequence
  primer for escherichia coli
gcgtcactttcctcttagtacaacagtgacgc

SEQ ID NO: 289 - DNA, 23 bp, Artificial Sequence
  primer for escherichia coli
aaccgctgttgctaacagaactg

SEQ ID NO: 290 - DNA, 27 bp, Artificial Sequence
  primer for escherichia coli
gcagcattccggcacaggtaatgctgc

SEQ ID NO: 291 - DNA, 19 bp, Artificial Sequence
  primer for escherichia coli
acgcggtaaaagtgccaga

SEQ ID NO: 292 - DNA, 27 bp, Artificial Sequence
  primer for escherichia coli
acagcattccggcacaggtaatgctgt

SEQ ID NO: 293 - DNA, 27 bp, Artificial Sequence
  primer for escherichia coli
cgaacggtggacatcaacagccgttcg

SEQ ID NO: 294 - DNA, 19 bp, Artificial Sequence
  primer for escherichia coli
cagcttgtactgctcgcca

SEQ ID NO: 295 - DNA, 27 bp, Artificial Sequence
  primer for escherichia coli
agaacggtggacatcaacagccgttct

SEQ ID NO: 296 - DNA, 24 bp, Artificial Sequence
  primer for escherichia coli
cgagttcccatggcgcggaactcg

SEQ ID NO: 297 - DNA, 20 bp, Artificial Sequence
  primer for escherichia coli
cctgatggcgagcagtacaa

SEQ ID NO: 298 - DNA, 24 bp, Artificial Sequence
  primer for escherichia coli
tgagttcccatggcgcggaactca

SEQ ID NO: 299 - DNA, 32 bp, Artificial Sequence
  primer for escherichia coli
tgtactgacgctttttcacgctggtcagtaca

SEQ ID NO: 300 - DNA, 22 bp, Artificial Sequence
  primer for escherichia coli
cctcgggattggtgtctataaa

SEQ ID NO: 301 - DNA, 32 bp, Artificial Sequence
  primer for escherichia coli
ggtactgacgctttttcacgctggtcagtacc

SEQ ID NO: 302 - DNA, 32 bp, Artificial Sequence
  primer for escherichia coli
aatcaatgacacgagcacgtttgtcattgatt

SEQ ID NO: 303 - DNA, 24 bp, Artificial Sequence
  primer for escherichia coli
aggaactgctgtttggtaaaggta

SEQ ID NO: 304 - DNA, 32 bp, Artificial Sequence
  primer for escherichia coli
gatcaatgacacgagcacgtttgtcattgatc

SEQ ID NO: 305 - DNA, 26 bp, Artificial Sequence
  primer for escherichia coli
gcgatcggcccacagtttgcgatcgc

SEQ ID NO: 306 - DNA, 19 bp, Artificial Sequence
  primer for escherichia coli
gatcttgccgctttccaga

SEQ ID NO: 307 - DNA, 26 bp, Artificial Sequence
  primer for escherichia coli
tcgatcggcccacagtttgcgatcga

SEQ ID NO: 308 - DNA, 25 bp, Artificial Sequence
  primer for escherichia coli
tggttccagcgttttaccggaacca

SEQ ID NO: 309 - DNA, 23 bp, Artificial Sequence
  primer for escherichia coli
cgagttgggcaaaagtaacattc

SEQ ID NO: 310 - DNA, 25 bp, Artificial Sequence
  primer for escherichia coli
cggttccagcgttttaccggaaccg

SEQ ID NO: 311 - DNA, 29 bp, Artificial Sequence
  primer for escherichia coli
tacccagaagcaccagtatatgctgggta

SEQ ID NO: 312 - DNA, 18 bp, Artificial Sequence
  primer for escherichia coli
gccacaacgagagtggca

SEQ ID NO: 313 - DNA, 29 bp, Artificial Sequence
  primer for escherichia coli
cacccagaagcaccagtatatgctgggtg

SEQ ID NO: 314 - DNA, 37 bp, Artificial Sequence
  primer for escherichia coli
agttctggatgttataagtgcttgataatccagaact

SEQ ID NO: 315 - DNA, 26 bp, Artificial Sequence
  primer for escherichia coli
caataactgcttggattaaacagaca

SEQ ID NO: 316 - DNA, 37 bp, Artificial Sequence
  primer for escherichia coli
cgttctggatgttataagtgcttgataatccagaacg

SEQ ID NO: 317 - DNA, 29 bp, Artificial Sequence
  primer for escherichia coli
tacaaaaccgccaggaagagggttttgta

SEQ ID NO: 318 - DNA, 22 bp, Artificial Sequence
  primer for escherichia coli
agcagcgttctggagtatcaag

SEQ ID NO: 319 - DNA, 29 bp, Artificial Sequence
  primer for escherichia coli
cacaaaaccgccaggaagagggttttgtg

SEQ ID NO: 320 - DNA, 36 bp, Artificial Sequence
  primer for escherichia coli
accacgtaaccagttacacttatgtcattacgtggt

SEQ ID NO: 321 - DNA, 21 bp, Artificial Sequence
  primer for escherichia coli
aatgcgaaagccaaacttcct

SEQ ID NO: 322 - DNA, 36 bp, Artificial Sequence
  primer for escherichia coli
tccacgtaaccagttacacttatgtcattacgtgga

SEQ ID NO: 323 - DNA, 43 bp, Artificial Sequence
  primer for escherichia coli
taataccagttaccacgtaatgacataagtgtaactggtatta

SEQ ID NO: 324 - DNA, 21 bp, Artificial Sequence
  primer for escherichia coli
caccagcgcttaaatcaccac

SEQ ID NO: 325 - DNA, 43 bp, Artificial Sequence
  primer for escherichia coli
gaataccagttaccacgtaatgacataagtgtaactggtattc

SEQ ID NO: 326 - DNA, 22 bp, Artificial Sequence
  primer for escherichia coli
ccgcccctggctgacctggcgg

SEQ ID NO: 327 - DNA, 22 bp, Artificial Sequence
  primer for escherichia coli
tcatagcggtagcattggtttg

SEQ ID NO: 328 - DNA, 22 bp, Artificial Sequence
  primer for escherichia coli
tcgcccctggctgacctggcga

SEQ ID NO: 329 - DNA, 30 bp, Artificial Sequence
  primer for escherichia coli
gccgtacgctgttgcctttttaggtacggc

SEQ ID NO: 330 - DNA, 20 bp, Artificial Sequence
  primer for escherichia coli
tctgcagagccagaacgttg

SEQ ID NO: 331 - DNA, 30 bp, Artificial Sequence
  primer for escherichia coli
accgtacgctgttgcctttttaggtacggt

SEQ ID NO: 332 - DNA, 27 bp, Artificial Sequence
  primer for escherichia coli
tctacccagagttcagctgcgggtaga

SEQ ID NO: 333 - DNA, 20 bp, Artificial Sequence
  primer for escherichia coli
caggatctgcacaccaacgt

SEQ ID NO: 334 - DNA, 27 bp, Artificial Sequence
  primer for escherichia coli
gctacccagagttcagctgcgggtagc

SEQ ID NO: 335 - DNA, 31 bp, Artificial Sequence
  primer for escherichia coli
aaacggaaacggtactaacaccattccgttt

SEQ ID NO: 336 - DNA, 21 bp, Artificial Sequence
  primer for escherichia coli
ctcgccgattgcataataacg

SEQ ID NO: 337 - DNA, 31 bp, Artificial Sequence
  primer for escherichia coli
gaacggaaacggtactaacaccattccgttc

SEQ ID NO: 338 - DNA, 27 bp, Artificial Sequence
  primer for escherichia coli
taggcggattgcataataacgcgccta

SEQ ID NO: 339 - DNA, 20 bp, Artificial Sequence
  primer for escherichia coli
tggtgcgacattcagtgagc

SEQ ID NO: 340 - DNA, 27 bp, Artificial Sequence
  primer for escherichia coli
caggcggattgcataataacgcgcctg

SEQ ID NO: 341 - DNA, 28 bp, Artificial Sequence
  primer for escherichia coli
atcgcatcgctgctaatgcggatgcgat

SEQ ID NO: 342 - DNA, 19 bp, Artificial Sequence
  primer for escherichia coli
atccctgcccgtaatgacg

SEQ ID NO: 343 - DNA, 28 bp, Artificial Sequence
  primer for escherichia coli
gtcgcatcgctgctaatgcggatgcgac

SEQ ID NO: 344 - DNA, 31 bp, Artificial Sequence
  primer for escherichia coli
ggtgcacaattacgacaaagacgtgtgcacc

SEQ ID NO: 345 - DNA, 18 bp, Artificial Sequence
  primer for escherichia coli
ggcgaccatgcagtaacg

SEQ ID NO: 346 - DNA, 31 bp, Artificial Sequence
  primer for escherichia coli
agtgcacaattacgacaaagacgtgtgcact

SEQ ID NO: 347 - DNA, 32 bp, Artificial Sequence
  primer for escherichia coli
ctgctgtacgcgtgaaaaacctggtacagcag

SEQ ID NO: 348 - DNA, 23 bp, Artificial Sequence
  primer for escherichia coli
tgataataccaatgcacgctttc

SEQ ID NO: 349 - DNA, 32 bp, Artificial Sequence
  primer for escherichia coli
ttgctgtacgcgtgaaaaacctggtacagcaa

SEQ ID NO: 350 - DNA, 27 bp, Artificial Sequence
  primer for escherichia coli
acacgtttgagacaggccaaaacgtgt

SEQ ID NO: 351 - DNA, 20 bp, Artificial Sequence
  primer for escherichia coli
ggtcggcaaccctgtctatg

SEQ ID NO: 352 - DNA, 27 bp, Artificial Sequence
  primer for escherichia coli
gcacgtttgagacaggccaaaacgtgc

SEQ ID NO: 353 - DNA, 30 bp, Artificial Sequence
  primer for escherichia coli
acccatgattctgtcgataaactcatgggt

SEQ ID NO: 354 - DNA, 19 bp, Artificial Sequence
  primer for escherichia coli
ccctgggttatctggccat

SEQ ID NO: 355 - DNA, 30 bp, Artificial Sequence
  primer for escherichia coli
ccccatgattctgtcgataaactcatgggg

SEQ ID NO: 356 - DNA, 30 bp, Artificial Sequence
  primer for escherichia coli
aagctggacacttggttcatgctccagctt

SEQ ID NO: 357 - DNA, 30 bp, Artificial Sequence
  primer for escherichia coli
cagctggacacttggttcatgctccagctg

SEQ ID NO: 358 - DNA, 21 bp, Artificial Sequence
  primer for escherichia coli
cccgtactattcgtttgccga

SEQ ID NO: 359 - DNA, 30 bp, Artificial Sequence
  primer for escherichia coli
tatacgcaagaatccaccaggttgcgtata

SEQ ID NO: 360 - DNA, 22 bp, Artificial Sequence
  primer for escherichia coli
ggtagagaagtttgacccggaa

SEQ ID NO: 361 - DNA, 30 bp, Artificial Sequence
  primer for escherichia coli
catacgcaagaatccaccaggttgcgtatg

SEQ ID NO: 362 - DNA, 27 bp, Artificial Sequence
  primer for escherichia coli
ggttcgctgaacgtttacctgcgaacc

SEQ ID NO: 363 - DNA, 22 bp, Artificial Sequence
  primer for escherichia coli
gtccagcttatgggacaactca

SEQ ID NO: 364 - DNA, 27 bp, Artificial Sequence
  primer for escherichia coli
tgttcgctgaacgtttacctgcgaaca

SEQ ID NO: 365 - DNA, 26 bp, Artificial Sequence
  primer for escherichia coli
tgccttggttgcaactggacaaggca

SEQ ID NO: 366 - DNA, 20 bp, Artificial Sequence
  primer for escherichia coli
agaggtgcggattcaccact

SEQ ID NO: 367 - DNA, 26 bp, Artificial Sequence
  primer for escherichia coli
ccccttggttgcaactggacaagggg

SEQ ID NO: 368 - DNA, 26 bp, Artificial Sequence
  primer for escherichia coli
tgggactcaccacttgcaaagtccca

SEQ ID NO: 369 - DNA, 19 bp, Artificial Sequence
  primer for escherichia coli
gaactgcgtgatgcggatc

SEQ ID NO: 370 - DNA, 26 bp, Artificial Sequence
  primer for escherichia coli
cgggactcaccacttgcaaagtcccg

SEQ ID NO: 371 - DNA, 32 bp, Artificial Sequence
  primer for escherichia coli
ggacagagtcgggtagatatcacactctgtcc

SEQ ID NO: 372 - DNA, 24 bp, Artificial Sequence
  primer for escherichia coli
cgggtgaaggttatctctatgaac

SEQ ID NO: 373 - DNA, 32 bp, Artificial Sequence
  primer for escherichia coli
agacagagtcgggtagatatcacactctgtct

SEQ ID NO: 374 - DNA, 26 bp, Artificial Sequence
  primer for escherichia coli
ggtccgcggttgtaataggtcggacc

SEQ ID NO: 375 - DNA, 21 bp, Artificial Sequence
  primer for escherichia coli
ggtgatggcgtgattgtctta

SEQ ID NO: 376 - DNA, 26 bp, Artificial Sequence
  primer for escherichia coli
cgtccgcggttgtaataggtcggacg

SEQ ID NO: 377 - DNA, 23 bp, Artificial Sequence
  primer for escherichia coli
gctgggaacggccagcacccagc

SEQ ID NO: 378 - DNA, 18 bp, Artificial Sequence
  primer for escherichia coli
cacgattttgtgctgcgc

SEQ ID NO: 379 - DNA, 23 bp, Artificial Sequence
  primer for escherichia coli
actgggaacggccagcacccagt

SEQ ID NO: 380 - DNA, 29 bp, Artificial Sequence
  primer for escherichia coli
gctgtttgttgatgcagctgacaaacagc

SEQ ID NO: 381 - DNA, 19 bp, Artificial Sequence
  primer for escherichia coli
tttccataacgcacgcgag

SEQ ID NO: 382 - DNA, 29 bp, Artificial Sequence
  primer for escherichia coli
actgtttgttgatgcagctgacaaacagt

SEQ ID NO: 383 - DNA, 23 bp, Artificial Sequence
  primer for escherichia coli
tattcccrggartttaygataga

SEQ ID NO: 384 - DNA, 18 bp, Artificial Sequence
  primer for escherichia coli
atccrgagcctgatkcac

(19) United States
(12) Patent Application Publication
(10) Pub. No.: US 2010/0279294 A1
(43) Pub. Date: Nov. 4, 2010

(54) METHODS OF DETECTING AND GENOTYPING ESCHERICHIA COLI O157:H7

(76) Inventors: THOMAS WHITTAM, Okemos, MI (US); Shannon Manning, Howell, MI (US); David Alland, Bernardsville, NJ (US); Alifiya Motiwala, Bayonne, NJ (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/718,668

(22) Filed: Mar. 5, 2010

Related U.S. Application Data
(60) Provisional application No. 61/158,633, filed on Mar. 9, 2009.

(51) Int. Cl. C12Q 1/68

(52) U.S. Cl. 435/6

(57) ABSTRACT

A method for detecting and genotyping Escherichia coli O157:H7 strains, including detecting nucleotides at single nucleotide polymorphism (SNP) loci, the identity of which nucleotides define SNP genotypes. A method for genotyping E. coli O157:H7 strains, including detecting thirty-two nucleotides at thirty-two single nucleotide polymorphism (SNP) loci, the identity of which nucleotides define thirty-six SNP genotypes. Multiplexed primer trios capable of detecting the nucleotides at E. coli SNP loci, and a kit including one or more primer trios.

This application claims benefit of provisional application Ser. No. 61/158,633, filed Mar. 9, 2009, entitled “Methods of Detecting and Genotyping Escherichia coli O157:H7”, the entire contents of which are incorporated herein in their entirety.

### STATEMENT REGARDING FEDERALLY SPONSORED RESEARCH OR DEVELOPMENT

This invention was in part made with United States government support awarded by the following agency: National Institute of Health/NIAID grant number N01 AI30058 and NIH grant AI049353. The United States has certain rights in this invention.

### BACKGROUND OF THE INVENTION

Enterohemorrhagic Escherichia coli (EHEC) includes a diverse population of Shiga toxin-producing E. coli that causes outbreaks of food and waterborne disease (1-3). EHEC often resides in bovine reservoirs and is transmitted via many food vehicles including cooked meat, such as hamburger (4) and salami (5) and raw vegetables, such as lettuce (6, 7) and spinach (8). In North America, E. coli O157:H7 is the most common EHEC serotype contributing to more than 75,000 human infections (9) and 17 outbreaks (3) per year.

The population genetics and epidemiology of E. coli O157:H7 infections have changed dramatically since the first outbreaks of illness associated with contaminated ground beef occurred in the early 1980s (1). New routes of infection, including direct contact with animals, and survival in novel food vehicles, particularly fresh produce, have become major sources of new disease cases and have contributed to widespread epidemics (3). This changing epidemiology is also influenced by the genetic variation and “relentless evolution” (41) of the O157 pathogen population. As the population of EHEC O157 strains has increased in frequency and spread geographically, it has genetically diversified. Isolates of EHEC O157 from clinical and bovine sources have been shown to be genotypically diverse by different methods, including pulsed field gel electrophoresis (PFGE) (26), octomer based genome scanning (42), and multilocus variable number of tandem repeats analysis (MLVA) (43). Studies of prophage and prophage remnants in EHEC O157 strains have indicated that genotypic diversity is largely attributable to bacteriophage-related insertions, deletions, and duplications of variable sizes of DNA fragments (24, 25, 44).

Substantial variability in clinical presentation also has been observed among patients with EHEC O157 infections. This variation is even apparent among different O157 outbreaks, as some outbreaks have contributed to remarkably high frequencies of HUS and hospitalization relative to others (Table 1). Consequently, it appears that there is extensive variation in virulence among distinct clades of O157.

TABLE 1
SG and clade for several E. coli 0157:H7 outbreak strains with hospitalization and HUS rates by outbreak

| | | | | | | No. of | | |
|---|---|---|---|---|---|---|---|---|
| | | | | | | hospitalizations | No. of | |
| Strain* | Year | SG | Clade | Outbreak | No. of cases | (%) | HUS (%) | Ref(s). |
| Sakai† | 1996 | 1 | 1 | Radish sprouts, | 5,000-12,680 | 398-425 (3-5) | 0-122 (0-3) | 13-15 |
| | | | | Sakai, Japan | | | | |
| 93-111 | 1993 | 9 | 2 | Hamburger, | 583 | 171 (29) | 41 (7) | 4 |
| | | | | northwest U.S. | | | | |
| EDL-933 | 1982 | 12 | 3 | Hamburger, | 47 | 33 (70) | 0 (0) | 36 |
| | | | | Michigan and | | | | |
| | | | | Oregon | | | | |
| TW14359 | 2006 | 30 | 8 | Spinach, | 204 | 104 (51) | 31 (15) | 37 |
| | | | | western U.S. | | | | |
| TW14588 | 2006 | 30 | 8 | Lettuce, eastern | 71 | 53 (75) | 8 (11) | 7 |
| | | | | U.S. | | | | |

| | | | | |
|---|---|---|---|---|
| 350 O157 outbreaks in the U.S. (1982-2002) | 8,598 | 1,493 (17) | 354 (4) | 3 |

*Sakai (RIMD-0509952) and EDL-933 have complete genome sequence available, and strain TW14359 has been sequenced by pyrosequencing (see text).
†The range is reported for the number of cases and frequency of HUS and hospitalization in the Sakai outbreak because the numbers vary in the literature.

It is not clear why outbreaks of EHEC O157 vary dramatically in the severity of illness and the frequency of the most serious complication, hemolytic uremic syndrome (HUS) (10-12). The 1993 outbreak in western North America (4) and the large 1996 outbreak in Japan (13) had low rates of hospitalization and HUS (14, 15), whereas the 2006 North American spinach outbreak (8) had high rates of both hospitalization (>50%) and HUS (>10%). One hypothesis is that outbreak strains differ in virulence as a result of variation in the presence and expression of different Shiga toxin (Stx) gene combinations (16-19).

Although molecular subtyping methods, such as PFGE, reveal extensive genomic diversity among O157 outbreaks, “DNA fingerprinting” data are not amenable to population genetic or phylogenetic analyses. PFGE analysis has demonstrated that differences between O157 strains result from discrete insertions or deletions that contribute to restriction site changes between strains rather than SNPs (24). Comparison of multiple O157 genomes has shown that bacteriophage variation is a major factor in generating genomic diversity (25) and presumably underlies most genomic variability detected by PFGE (24, 26).

### BRIEF SUMMARY OF THE INVENTION

The inventors have developed primers for use in a method for genotyping E. coli O157:H7 by detecting the nucleotides at 96 single nucleotide polymorphism (SNP) loci in E. coli O157:H7, and applying this method to more than 500 E. coli O157:H7 clinical strains. Phylogenetic analyses identified 39 SNP genotypes (SGs) that differ at 20% of SNP loci and are separated into nine distinct clades. Differences were observed between clades in the frequency and distribution of Shiga toxin genes and in the type of clinical disease reported. Patients with hemolytic uremic syndrome (HUS) were significantly more likely to be infected with clade 8 strains, which have increased in frequency over the past 5 years. Genome sequencing of a spinach outbreak strain, a member of clade 8, also revealed substantial genomic differences. The present method suggests that an emergent subpopulation of the clade 8 lineage has acquired critical factors that contribute to more severe disease.

More specifically, the present invention includes methods for detecting E. coli O157:H7 strains. The present invention further includes detecting E. coli O157:H7 strains in any of 36 SNP genotypes using multiplexed primer sets that are capable of identifying 32 SNPs. In one embodiment, these methods are used to detect E. coli O157:H7 strains with increased virulence, e.g., E. coli O157:H7 strains that are or would be included in clade 8, as defined herein.

The present invention also includes methods for diagnosing diseases caused by E. coli O157:H7 infections. In one embodiment, these methods are used to diagnose diseases associated with infection by E. coli O157:H7 strains that may have increased virulence, e.g., E. coli O157:H7 strains from clade 8, as defined herein.

The present invention includes a method for genotyping E. coli O157:H7, including providing a sample of DNA from a possible E. coli O157:H7 infection; detecting in the sample whether the identity of the nucleotide at position 125 of SEQ ID NO. 11 is thymine (T) or guanine (G), the nucleotide at position 648 of SEQ ID NO. 82 is T or cytosine (C), the nucleotide at position 299 of SEQ ID NO. 47 is T or C, the nucleotide at position 339 of SEQ ID NO. 15 is T or C, the nucleotide at position 144 of SEQ ID NO. 67 is adenine (A) or G, the nucleotide at position 417 of SEQ ID NO. 78 is T or C, the nucleotide at position 3971 of SEQ ID NO. 52 is G or T, the nucleotide at position 1186 of SEQ ID NO. 75 is C or G, the nucleotide at position 2244 of SEQ ID NO. 81 is T or C, the nucleotide at position 1151 of SEQ ID NO. 10 is T or C, the nucleotide at position 1678 of SEQ ID NO. 16 is G or C, the nucleotide at position 1545 of SEQ ID NO. 17 is G or A, the nucleotide at position 311 of SEQ ID NO. 21 is G or A, the nucleotide at position 1340 of SEQ ID NO. 48 is G or A, the nucleotide at position 776 of SEQ ID NO. 35 is G or A, the nucleotide at position 132 of SEQ ID NO. 57 is G or T, the nucleotide at position 348 of SEQ ID NO. 46 is A or C, the nucleotide at position 928 of SEQ ID NO. 20 is G or A, the nucleotide at position 849 of SEQ ID NO. 36 is G or A, the nucleotide at position 247 of SEQ ID NO. 79 is G or A, the nucleotide at position 83 of SEQ ID NO. 1 is T or C, the nucleotide at position 117 of SEQ ID NO. 6 is C or A, the nucleotide at position 259 of SEQ ID NO. 22 is C or T, the nucleotide at position 379 of SEQ ID NO. 18 is C or T, the nucleotide at position 739 of SEQ ID NO. 4 is G or A, the nucleotide at position 527 of SEQ ID NO. 47 is C or T, the nucleotide at position 693 of SEQ ID NO. 74 is C or T, the nucleotide at position 281 of SEQ ID NO. 11 is C or T, the nucleotide at position 267 of SEQ ID NO. 57 is G or A, the nucleotide at position 2707 of SEQ ID NO. 66 is C or A, the nucleotide at position 354 of SEQ ID NO. 47 is C or A, and the nucleotide at position 339 of SEQ ID NO. 70 is T or A; and using the identities of these nucleotides to determine whether the possible E. coli O157:H7 has a particular single nucleotide polymorphism (SNP) genotype (SG) of an E. coli O157:H7 that is defined by these nucleotides.

The invention also includes the above method wherein the identity of the nucleotide at position 125 of SEQ ID NO. 11 is G, the nucleotide at position 648 of SEQ ID NO. 82 is C, the nucleotide at position 299 of SEQ ID NO. 47 is C, the nucleotide at position 339 of SEQ ID NO. 15 is C, the nucleotide at position 144 of SEQ ID NO. 67 is G, the nucleotide at position 417 of SEQ ID NO. 78 is C, the nucleotide at position 3971 of SEQ ID NO. 52 is T, the nucleotide at position 1186 of SEQ ID NO. 75 is G, the nucleotide at position 2244 of SEQ ID NO. 81 is T, the nucleotide at position 1151 of SEQ ID NO. 10 is C, the nucleotide at position 1678 of SEQ ID NO. 16 is G, the nucleotide at position 1545 of SEQ ID NO. 17 is G, the nucleotide at position 311 of SEQ ID NO. 21 is G, the nucleotide at position 1340 of SEQ ID NO. 48 is A, the nucleotide at position 776 of SEQ ID NO. 35 is A, the nucleotide at position 132 of SEQ ID NO. 57 is G, the nucleotide at position 348 of SEQ ID NO. 46 is A, the nucleotide at position 928 of SEQ ID NO. 20 is G, the nucleotide at position 849 of SEQ ID NO. 36 is G, the nucleotide at position 247 of SEQ ID NO. 79 is G, the nucleotide at position 83 of SEQ ID NO. 1 is C, the nucleotide at position 117 of SEQ ID NO. 6 is C, the nucleotide at position 259 of SEQ ID NO. 22 is C or T, the nucleotide at position 379 of SEQ ID NO. 18 is C or T, the nucleotide at position 739 of SEQ ID NO. 4 is G or A, the nucleotide at position 527 of SEQ ID NO. 47 is C or T, the nucleotide at position 693 of SEQ ID NO. 74 is C or T, the nucleotide at position 281 of SEQ ID NO. 11 is T, the nucleotide at position 267 of SEQ ID NO. 57 is G, the nucleotide at position 2707 of SEQ ID NO. 66 is C, the nucleotide at position 354 of SEQ ID NO. 47 is C, and the nucleotide at position 339 of SEQ ID NO. 70 is T; and the possible E. coli O157:H7 is determined to have a SG of an E. coli O157:H7 genotype associated with more severe disease.

With the inventive method, the SG determination may be used to identify the strain or the clade of E. coli O157:H7 for use in large-scale epidemiological studies; or the SG determination may be used as a tool to diagnose infection by E. coli O157:H7 in a clinical setting. Further, the inventive method may be used to test a sample from a plant or animal, including a human, to determine whether E. coli is present by screening for the SG and possibly, other identifying genetic characteristics in any given sample.

The inventive method also can involve the use of real-time polymerase chain reaction (PCR) assays to detect the nucleotides at each of the SNP loci together or individually. Primer trios may be used in the PCR assay, and the primer trios may be selected from the oligonucleotides identified by SEQ ID NOs. 83-382 herein.

Finally, the inventive method also includes identifying the organism in the sample as having one of thirty-nine SGs that are defined by the above-described nucleotides at the SNP loci.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings and tables, certain embodiment(s) which are presently preferred. It should be understood, however, that the invention is not limited to the precise arrangements and instrumentalities shown.

FIGS. 1A-1C show the genetic relatedness of E. coli O157 among 403 O157 and closely related O55:H7 strains based on 96 single nucleotide polymorphisms (SNPs). FIG. 1A shows the location of 83 genes within 96 SNP loci on the E. coli O157:H7 genomic map of the Sakai strain. Real time PCR assays detected 52 loci with non-synonymous (black circles) and 43 with synonymous (white circles) polymorphisms, and one locus (uidA-686) with a GG insertion (open triangle). FIG. 1B shows the distribution of nucleotide diversity across 96 SNP loci. Diversity ranges from 0 for two monomorphic SNP loci to a maximum between 0.45-0.50 for 26 loci. The average nucleotide diversity for the 96 loci is 0.212±0.199. FIG. 1C shows the phylogenetic relationships among SNP genotypes (SGs) using the minimum evolution algorithm based on the distance matrix of pairwise differences between SGs. The consensus tree is shown with the percentages at the nodes of the >70% bootstrap confidence values based on 1000 replicates. Both the GUD+ and Sor+, which occur in the clade 9, are negative (GUD− and Sor−) in the derived clades 1-8.

FIG. 2 shows the phylogenetic network applied to 48 parsimoniously informative (PI) sites using the Neighbor-net algorithm for 528 E. coli O157 strains. The colored ellipses mark clades supported in the minimum evolution phylogeny. The numbers at the nodes denote the SNP genotypes (SGs) 1 to 39, and the white circle nodes contain two SGs that match at the 48 PI sites. The seven SGs found among multiple continents are marked with squares.

FIGS. 3A and 3B show the distribution of Shiga toxin (Stx) genes in E. coli O157 clades. FIG. 3A shows the frequency of 528 O157 strains that were classified into one of 9 clades based on SNP genotyping, ranked from left to right in the histogram by decreasing frequency. The four most common clades were clades 2 (47.6%), 8 (25.4%), 3 (10.6%), and 7 (7.3%). FIG. 3B shows the distribution of Shiga toxin gene variants (stx1, stx2, and stx2c) among 519 of the 528 O157 strains organized into 9 clades. The percentage of PCR-assay positive strains overall is given in parentheses.

FIG. 4 shows odd ratios with 95% confidence intervals (dotted lines) highlighting the association between patient characteristics and infection with specific clades. Logistic regression models were adjusted for age, gender, bloody diarrhea, diarrhea, abdominal pain, chills, HUS, hospitalization, and body aches. Dark circles show significant associations.

FIG. 5 shows a circular map of the E. coli Sakai complete genome and comparisons with the spinach outbreak strain partial genome and the EDL-933 complete genome. The outer two circles show Sakai protein coding genes colored by Clusters of Orthologous Groups (COGs) of proteins (52). Genes on the forward strand are shown by the outside circle, and genes on the reverse strand are shown by the inside circle. In circles 3 and 4, Sakai genes conserved in EDL-933 are in blue; non-conserved genes are in grey. In circles 5 and 6, Sakai genes conserved in the spinach strain are in gold; non-conserved genes are in grey. Circles 7 and 8 show Sakai genes containing SNPs in EDL-933. Circles 9 and 10 show Sakai genes containing SNPs in the spinach strain. These SNP harboring genes are colored by the number of SNPs: 1-5 SNPs in green; 6-10 SNPs in blue; 11-20 SNPs in orange; >20 SNPs in red. The number of highly conserved genes (n=2,741) is highlighted among three O157 genomes. The Sakai and EDL-933 genomes are more similar to each other in gene content and nucleotide sequence identity (3.2%) than to the clade 8 spinach outbreak strain (10.65 or 10.7%).

FIG. 6 shows year by year changes in the number of reported cases of E. coli O157:H7 in Michigan (n=444). The decrease in the annual number of cases in Michigan from 2002 follows the national trend in E. coli O157:H7 disease (dotted line identified as “Total”). The percentage of strains representing clade 8 has increased in frequency over time (solid line), whereas clade 2 frequency has decreased (dashed line identified as “Clade 2”).

### DETAILED DESCRIPTION OF THE PREFERRED EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention. The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, Examples, Sequence Listing, and from the claims. The preferred embodiments of the present invention may be understood more readily by reference to the following detailed description of the specific embodiments, the Examples, and the Sequence Listing included hereafter.

The text file filed concurrently with this application, titled “MIC037P349 Sequence Listing.txt” contains material identified as SEQ ID NOS: 1-384 which material is incorporated herein by reference. This text file was created on Mar. 5, 2010, and is 218,851 bytes.

For clarity of disclosure, and not by way of limitation, the detailed description of the invention is divided into the subsections that follow.

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and nucleic acid chemistry described below are those well known and commonly employed in the art. Although any methods, devices and materials similar or equivalent to those described herein can be used in the practice or testing of the invention, the preferred methods, devices and materials are now described.

In this specification and the appended claims, the singular forms “a,” “an” and “the” include plural reference unless the context clearly dictates otherwise.

The inventors genotyped more than 500 clinical strains of EHEC O157 based on 96 SNPs that separated strains into genetically distinct groups, and sequenced the genome of the O157 strain implicated in the spinach outbreak. These data form a basis for addressing how EHEC O157 has diversified and evolved in genome content, and for assessing intrinsic differences among O157 lineages with regard to clinical presentation and disease severity.

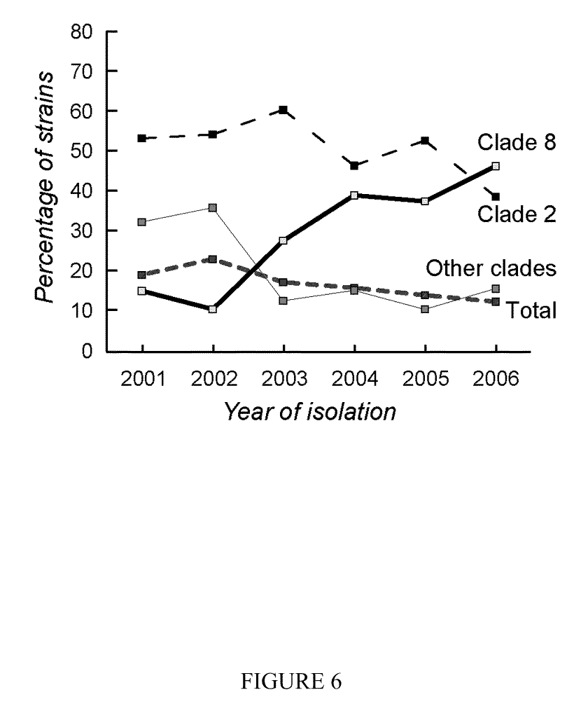

The evaluation of more than 500 O157 strains from clinical sources for up to 96 SNP loci highlights the degree of genetic variation among strains, and identifies a specific O157 lineage (clade 8) that has increased in frequency (FIG. 6). This increase in clade 8 is surprising given that at the same time, the overall national prevalence of EHEC O157 infections has been decreasing (45). Strains of the clade 8 lineage have caused two recent and unusually severe outbreaks linked to produce, are associated with HUS, and more frequently carry both the stx2 and stx2c genes. In concert, these results suggest that a more virulent subpopulation of EHEC O157 is increasing in its contribution to the overall disease burden associated with O157 infections. Although there are clear differences in the frequency and combination of stx genes among clades, the toxin-gene combination alone does not account for the variation in hospitalization and HUS rates by clade.

The observation that clade 8 strains more frequently have both the stx2 and stx2c genes infers that carriage of both the Stx2 and Stx2c phages contribute in part to the greater virulence of clade 8 strains. The Stx genes, encoded by lambda-like bacteriophages, can circulate among hundreds of different E. coli strains, (46) and integrate into many sites in the O157 genome (25, 44). Previous studies have observed correlations between specific Stx genes and disease, particularly for stx2 and stx2c (18, 19), though it has not been suggested that having both variants together may increase virulence. Because not all clade 8 strains have both stx2 and stx2c, and none of the strains have only stx2c, the presence and presumable production of the Stx2c variant alone cannot be solely responsible for the enhanced virulence attributed to this lineage. This also is true for the production of Stx2, as it was detected in nearly every strain representing all nine clades. We cannot, however, rule out the possibility that stx2c is rapidly lost during infection, thereby inhibiting our ability to detect it in some strains. What accounts for the greater intrinsic virulence among clade 8 strains and other O157 genotypes has not been fully understood. There is a constellation of mobile genetic elements that contribute to the virulence of pathogenic E. coli (47), and it is possible that a novel combination of virulence factors has emerged in the clade 8 lineage.

Among the three most common clades (2, 7, and 8) examined, there are noteworthy differences in transmission and clinical disease characteristics (Table 2) in addition to the association between clade 8 and HUS.

TABLE 2

| | Clade 8 (n = 63)* | | | | Clade 2 (n = 154)* | | | | Clade 7 (n = 31)* | | | |
|---|---|---|---|---|---|---|---|---|---|---|---|---|
| Characteristic† | n | (%) | OR (95% CI) | P | n | (%) | OR (95% CI) | P | n | (%) | OR (95% CI) | P |
| Bloody | | | | | | | | | | | | |
| diarrhea | | | | | | | | | | | | |
| No (n = 57) | 8 | (14) | 1.0 | | 25 | (43) | 1.0 | | 16 | (28) | | |
| Yes (n = 234) | 55 | (24) | 1.8 (0.84, 4.21) | .11 | 129 | (55) | 1.6 (0.88, 2.81) | .13 | 15 | (6) | 0.2 (0.08, 0.38) | <.0001 |
| Non-bloody | | | | | | | | | | | | |
| diarrhea | | | | | | | | | | | | |
| No (n = 112) | 23 | (21) | 1.0 | | 64 | (57) | 1.0 | | 13 | (12) | | |
| Yes (n = 179) | 40 | (22) | 1.1 (0.62, 1.98) | .71 | 90 | (50) | 0.8 (0.47, 1.22) | .25 | 18 | (10) | 0.9 (0.40, 1.81) | .68 |
| Abdominal pain | | | | | | | | | | | | |
| No (n = 52) | 7 | (13) | 1.0 | | 26 | (50) | 1.0 | | 8 | (15) | 1.0 | |
| Yes (n = 239) | 56 | (23) | 2.0 (0.84, 4.61) | .10 | 128 | (54) | 1.2 (0.63, 2.10) | .64 | 23 | (10) | 0.6 (0.25, 1.39) | .24 |
| Body aches | | | | | | | | | | | | |
| No (n = 244) | 53 | (22) | 1.0 | | 126 | (52) | 1.0 | | 26 | (11) | 1.0 | |
| Yes (n = 47) | 10 | (21) | 1.0 (0.45, 2.09) | .95 | 28 | (60) | 1.4 (0.73, 2.60) | .32 | 5 | (11) | 1.0 (0.36, 2.75) | 1.0 |
| HUS | | | | | | | | | | | | |
| No (n = 281) | 56 | (20) | 1.0 | | 151 | (54) | 1.0 | | 31 | (11) | NA | |
| Yes (n = 10) | 7 | (70) | 9.4 (2.35, 37.41) | .0008 | 3 | (30) | 0.4 (0.09, 1.46) | .14 | 0 | (0) | NA | .13 |
| Chills | | | | | | | | | | | | |
| No (n = 230) | 44 | (19) | 1.0 | | 124 | (54) | 1.0 | | 24 | (10) | 1.0 | |
| Yes (n = 60) | 19 | (32) | 2.0 (1.04, 3.70) | .04 | 29 | (48) | 0.8 (0.45, 1.41) | .44 | 7 | (12) | 1.1 (0.46, 2.77) | .79 |
| Hospitalization | | | | | | | | | | | | |
| No (n = 147) | 27 | (18) | 1.0 | | 78 | (53) | 1.0 | | 17 | (12) | 1.0 | |
| Yes (n = 147) | 37 | (25) | 1.5 (0.85, 2.62) | .16 | 77 | (52) | 1.0 (0.62, 1.54) | .91 | 14 | (10) | 0.8 (0.38, 1.70) | .57 |
| Age (years) | | | | | | | | | | | | |
| 0-18 (n = 148) | 37 | (25) | 1.0 | | 76 | (51) | 1.0 | | 14 | (9) | 1.0 | |
| 19-64 (n = 172) | 32 | (19) | 0.7 (0.41, 1.17) | .16 | 93 | (54) | 1.1 (0.72, 1.73) | .63 | 20 | (12) | 1.3 (0.61, 2.59) | .53 |
| Gender | | | | | | | | | | | | |
| Female | 40 | (23) | 1.0 | | 78 | (46) | 1.0 | | 24 | (14) | 1.0 | |
| (n = 171) | | | | | | | | | | | | |
| Male (n = 149) | 29 | (19) | 0.8 (0.46, 1.36) | .39 | 91 | (61) | 1.9 (1.20, 2.92) | .006 | 10 | (7) | 0.4 (0.20, 0.95) | .03 |

As to Table 2, there are crude associations between patient characteristics and infection with E. coli O157 strains (n=333) of different clades. Differences in the distribution of clades as measured by clinical data and bacterial characteristics were tested using the Likelihood Ratio Chi square (1 degree of freedom); odds ratios (OR), 95% confidence intervals (95% CI), and P values (P) were obtained based on these distributions. * means percentages and associations are relative to all other clades combined; clade 9 strains were omitted from the analysis. Only 1 strain per outbreak or cluster was used in the analyses. † means number varies depending on characteristic as some data were missing.

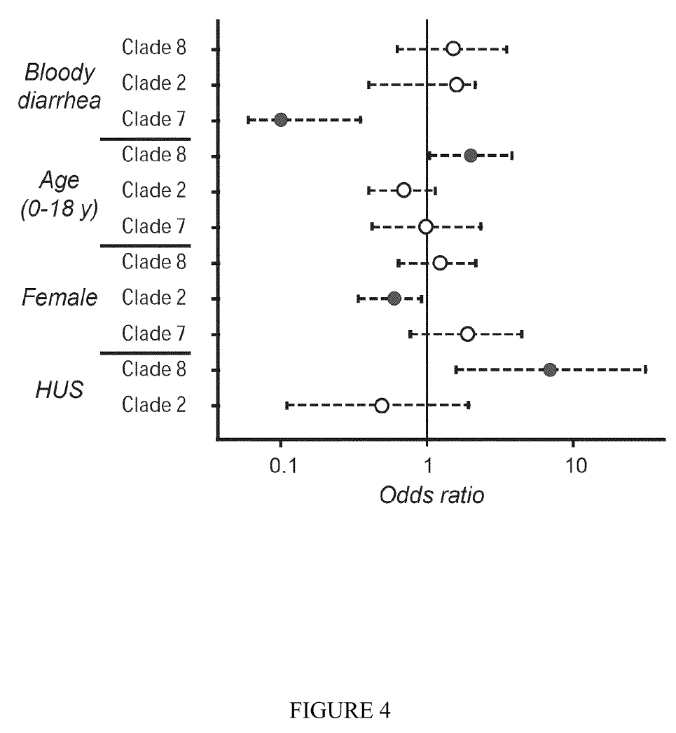

For example, patients infected with strains from both clades 2 and 8 reported bloody diarrhea more frequently when compared to patients with clade 7 infections. Furthermore, clades 7 and 8 were more common among female patients, and clade 8 was associated with disease in younger (<18 yrs) patients (FIG. 4). These observed differences among patients with O157 infections clearly reflect differences among the common clades that can result from variability in gene content or genetic variation in conserved, common genes. The sequence comparisons of the spinach outbreak genome (clade 8) with the two other complete genomes (clades 1 and 3) indicate that there has been sufficient evolution time for 5% mutational substitution (10% differences in sequence of 2,741 conserved genes). This is consistent with a study by Zhang et al. (23) that estimated the most recent ancestor for EHEC O157 strains in clades 1 through 8 (β-glucuronidase-negative, non-sorbitol-fermenting) to be between 32.7 and 34.3 thousand years ago.

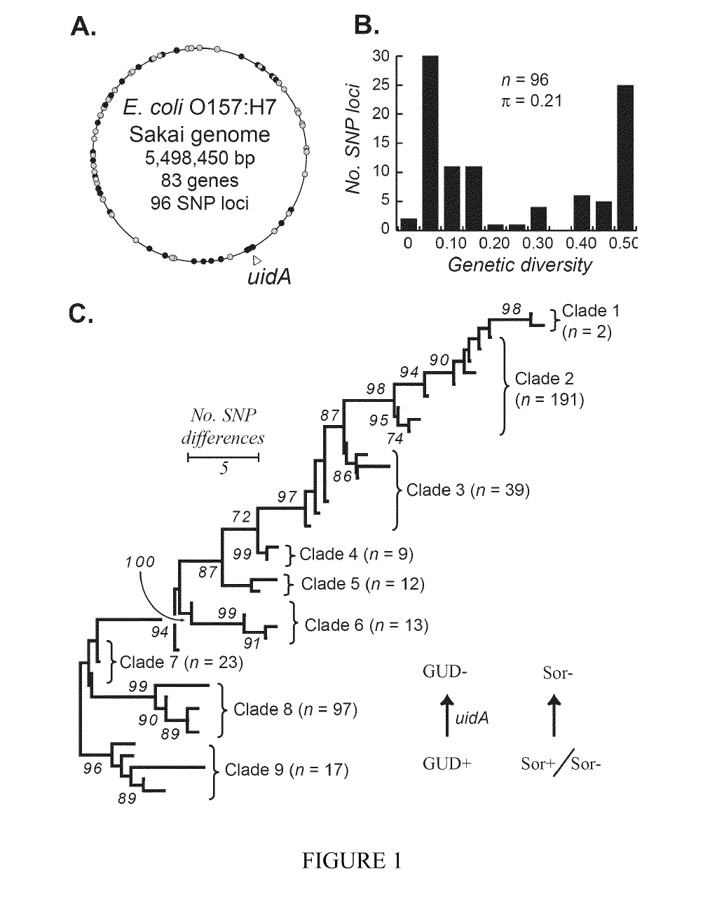

To determine when specific clades first appeared in human disease and assess whether clade 8 strains have increased in frequency in strains recovered from outside of Michigan, the inventors evaluated a subset of O157 strains isolated during different time periods. Through this screening, the inventors identified clade 8 strains from clinical cases dating back to 1984 on multiple continents (Table 3) suggesting that clade 8 has not recently emerged. This result was confirmed by both the spinach outbreak genome (FIG. 4) and phylogenetic analyses (FIG. 1B), as clade 8 is more closely related to the evolutionarily ancestral O157 lineage (clade 9) than other lineages.

TABLE 3

| | | | | Freq. of |
|---|---|---|---|---|
| SG | Clade | SG geographic range | Date(s) | isolation |
| 1 | 1 | Japan, USA | 1996, 1998-2001 | 2 |
| 2 | 1 | Japan | 1996 | 1 |
| 3 | 2 | USA | 2001, 2002 | 2 |
| 4 | 2 | USA | 1998-2005 | 19 |
| 5 | 2 | USA | 2001, 2005 | 7 |
| 6 | 2 | USA | 2003 | 1 |
| 7 | 2 | USA | 1998, 2005 | 2 |
| 8 | 2 | USA | 1998-2006 | 12 |
| 9 | 2 | Japan, USA, Australia | 1988-2006 | 184 |
| 10 | 2 | USA | 2001-2006 | 20 |
| 11 | 2 | USA | 2002 | 1 |
| 12 | 3 | USA, Canada, Australia | 1982-2004 | 12 |
| 13 | 3 | USA | 1998-2004 | 15 |
| 14 | 3 | USA | 1999-2004 | 20 |
| 15 | 3 | USA | 2001 | 1 |
| 16 | 3 | USA | 1985-2001 | 4 |
| 17 | 3 | USA | 1994, 2001-2005 | 3 |
| 18 | 3 | Japan, USA | 1996, 2002 | 2 |
| 19 | 4 | USA | 2002-2003 | 8 |
| 20 | 4 | USA | 2002 | 2 |
| 21 | 5 | USA | 2002, 2006 | 2 |
| 22 | 5 | USA | 2004 | 1 |
| 23 | NA | USA | 2002 | 1 |
| 24 | 6 | USA | 2002 | 1 |
| 25 | 6 | USA, Australia | 1998-2005 | 9 |
| 26 | 6 | USA | 2001-2006 | 6 |
| 27 | 6 | USA | 2001 | 1 |
| 28 | 7 | USA | 2003 | 1 |
| 29 | 7 | USA, Canada | 1987-2006 | 37 |
| 30 | 8 | USA | 2000-2006 | 94 |
| 31 | 8 | USA, UK, Germany, | 1984-2003 | 9 |
| | | Argentina | | |
| 32 | 8 | USA | 2003 | 1 |
| 33 | 8 | USA, UK | 1998-2006 | 30 |
| 34 | 8 | USA | 1998 | 1 |
| 35* | 9 | USA | 1995-2004 | 7 |
| 36* | 9 | Germany | 1988-1991 | 6 |
| 37* | 9 | USA | 1995 | 1 |
| 38† | 9 | USA | 1979 | 1 |
| 39† | 9 | USA | 1994 | 1 |

Table 3 shows distribution and frequency of single nucleotide polymorphism (SNP) genotypes (SGs) among 528 E. coli O157 strains and close relatives. Strain isolation dates are represented by commas for SGs with less than two strains, and as a range for categories with more strains and those with an unknown collection date. * means SG-35 contains 7 strains including (β-glucuronidase positive, GUD+; sorbitol negative, Sor−) strains that are O157:H7. SG-36 contains 6 strains isolated in Germany that are GUD+/Sor+ and have serotype O157:H—. SG-37 strain represents a nontypeable (NT) serotype (O antigen) isolated from a healthy marmoset. † means strains are 055:H7 serotypes and represent the evolutionarily derived lineages (GUD−/Sor−).

In contrast to clade 8 strains from Michigan patients, the frequency of stx2c with or without stx2 did not increase in frequency over time, and stx2c was detected in a strain isolated in 1984, indicating that it too, has not recently emerged.

It is clear that EHEC O157 is genetically diversified and comprises multiple detectable clades with substantial genomic, biological, and epidemiological variation. SNP genotyping has revealed the clades that reflect the genetic variability among pathogenic strains associated with clinical infection. These results support the hypothesis that the clade 8 lineage has recently acquired novel factors that contribute to enhanced virulence. Evolutionary changes in the clade 8 subpopulation could explain its emergence in several recent foodborne outbreaks; however, it is not clear why this virulent subpopulation is increasing in prevalence. Since humans are more an incidental host for EHEC O157, further investigation of the bovine reservoir (48, 49) and environment is critical, as is the evaluation of agricultural practices in areas where livestock and produce are farmed side-by-side. Identifying the underlying factors that lead to enhanced virulence and the successful transmission of EHEC O157 in contaminated food and water is imperative. Similarly, conducting large-scale molecular epidemiologic studies is necessary to assess the actual distribution of SGs, clades and Stx variants in environmental reservoirs and broad geographic scales (50). The development and deployment of a rapid, inexpensive molecular test that can identify more virulent O157 subtypes also would be useful for clinical laboratories to identify patients with an increased likelihood of developing HUS.

The systematic analysis of SNPs is useful for E. coli outbreak investigations, can resolve closely related bacterial genotypes, provide insights into the micro-evolutionary history of genome divergence (20, 27), and contribute to an epidemiologic assessment of associations between bacterial genotypes and disease. Accordingly, to assess the genetic diversity and variability in virulence among E. coli O157 strains, the inventors developed a system for identifying synonymous and non-synonymous mutations as single nucleotide polymorphisms (“SNPs”) (20-23). In one embodiment, the system includes identifying the SNPs through the use of real time PCR. Other methods of identifying the polymorphic nucleotide will be understood by those of skill in the art.

The present invention includes a method for identifying a strain of E. coli O157:H7 by identifying the SNP genotype of the strain, including: (1) providing a sample of DNA from a possible E. coli O157:H7 infection; (2) detecting the nucleotides at a grouping or subset of SNP loci identified in Table 4 herein; (3) based on the nucleotide present at the SNP loci in the sample, identifying a SNP genotype (“SG”) for the sample (e.g., a SG selected from the SGs listed in Table 6 below); and, based on that SG, identifying the strain of E. coli O157:H7. In one embodiment, the SG is used to identify the clade, or phylogenetic lineage, of the strain (e.g., the clade is one of the nine clades identified in Table 6).

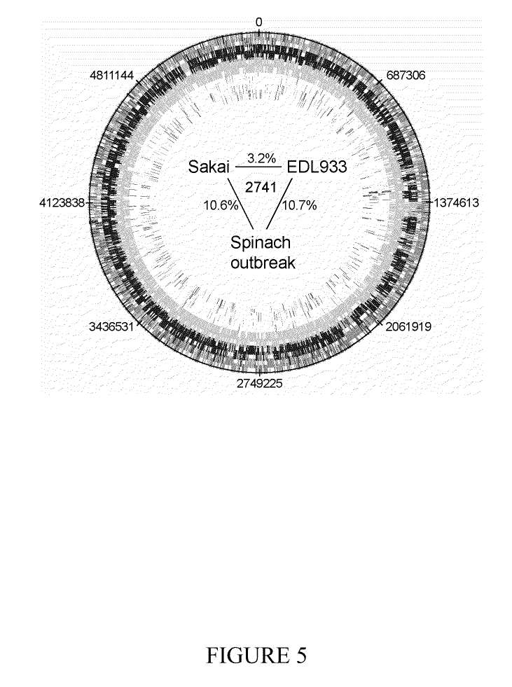

The O157 Sakai genome is used as a point of reference for identifying the location of the ninety-six SNPs of the present invention (Table 4) and this genome is comprised of 5,498,450 base pairs (see, Genbank Accession No. NC_{—}002695; as well as FIG. 5, hereto). For example, referring to Table 4 below, the SNP identified as “03_{—}83” is located at nucleotide position 351109 in the O157 Sakai genome. As further shown in Table 4, for example, the polymorphic SNP of “03_{—}83” includes a cytosine (C) instead of the thymine (T) at position 351109 of the O157 Sakai genome. The same system of identification is utilized for each of the other 95 SNPs.

The location of each of the SNPs of the present invention also is identified by its position within a gene of the O157 Sakai genome. For example, again referring to Table 4, the SNP identified as “03_{—}83” is located in gene (or open reading frame) “ECs0333” (SEQ ID NO. 1) at nucleotide position 83 of this gene. The same system of identification is utilized for the other 95 SNPs. SEQ ID NOs. 1-82 describe the nucleotide sequences for the genes (or ORFs) in which the 96 SNPs are located.

In addition to the detection methods described herein, other methods that could be used to detect the nucleotide at a SNP locus include real-time PCR, DNA sequencing and 454 pyrosequencing, which involves sequencing short stretches of DNA containing the SNPs (56).

In one embodiment of the invention, the nucleotides at the SNP loci are detected using real-time PCR. In this embodiment, primers are designed to detect a subset of the 96 SNPs identified in Table 4. For example, those primers may be one or more of the primer trios identified in Table 5 below. These primers have the nucleotide sequences identified in SEQ ID NOs. 83-382 and are used to detect the nucleotide at the SNP loci in the genes having the nucleotide sequences identified in SEQ ID NOs. 1-82. For example, the trio of primers having the nucleotide sequences of SEQ ID NOs. 86-88 can be used to detect the nucleotide at SNP position 83 in the gene having the nucleotide sequence of SEQ ID NO. 1. The primers are made according to methods known in the art and are used to detect the occurrence of the SNPs in a sample of DNA from a possible E. coli O157:H7 infection.

Based on the presence or absence of each of the SNPs in the sample, a SNP genotype can be identified for the sample (e.g., which SNP genotype may be selected from the SNP genotypes listed in Table 6 below); and, based on the SNP genotype, the clade of E. coli O157:H7 in the sample can be identified. For example, a sample can be identified as having the “SNP genotype 1” shown in Table 6 if the DNA of that sample includes all of the nucleotides identified for each of the 32 SNPs shown in the row of Table 6 identified as “1” under “SNP genotype” (i.e., if that DNA includes a thymine for SNP 03_{—}83, a guanine for SNP 95_{—}739, an adenine for SNP 09_{—}117, etc). The same process is used to identify whether an organism has any of the other 38 SNP genotypes shown in Table 6. Further, a sample can be identified as having the “SNP genotype 1” shown in Table 6 if the DNA of that sample includes all of the nucleotides identified for each of the 32 SNPs shown in the row of Table 6 identified as “1” under “SNP genotype(s)”, and the same process is used to identify each of the other 32 SNP genotypes shown in Table 6.

All 96 SNPs, or different groupings or subsets of the 96 SNPs can be used to identify a SNP genotype and, therefore, a strain of E. coli O157:H7. For example, one grouping of the 96 SNPs is the 32 SNPs identified in Table 6. Other groupings are the 32 SNPs identified in Table 6, all of the 96 SNPs identified in Table 4, or some other grouping of these 96 SNPs which can be used to identify a SNP genotype and, therefore, a strain of E. coli O157:H7. The groupings of 32 SNPs shown in Table 6 could be used for rapid detection for diagnostic or clinic applications. Additionally, all 96 SNPs identified in Table 4 could be used as a genotyping tool.

In one embodiment, nucleotides are detected at the 32 SNP loci shown in Table 6, and based on the occurrence of the nucleotides present at these positions, a determination is made whether the organism has any one of the thirty-six SNP genotypes described in Table 6. Note: in Table 6, in some instances, one SG is identified by more than one SG number, e.g., an SG is identified as both “4” and “6” (see also, SGs 16 and 17, as well as SG 20 and 23).

The methods of the present invention also include identifying an E. coli O157:H7 as belonging to one of the clades shown in Table 6 below. The methods of the present invention may be used to identify a strain of E. coli O157:H7 that either is known or unknown.

Having now generally described the invention, the same will be more readily understood through reference to the following examples, which are provided by way of illustration, and are not intended to be limiting of the present invention, unless specified.

### EXAMPLES

### Example 1

### Materials and Methods for Examples 2-8

Bacterial strains. A total of 528 EHEC O157 strains and close relatives were genotyped; 444 were from Michigan patients identified via surveillance by the Michigan Department of Community Health (MDCH), Bureau of Laboratories from 2001-2006 (40). Patients were confirmed to have O157-associated disease by culture, enzyme immunoassay, and real time PCR for stx1,2 (40). Strains with unique PFGE patterns or patterns present in 2 or fewer strains (n=333) were included in the epidemiological analyses. The additional 94 strains were selected based on epidemiological data to provide a sample representing different geographic locations and collection dates.

SNP loci and real time PCR assays. The 96 SNP loci (Table 4) were identified from data generated by comparative genome sequencing microarrays (23), multilocus sequence typing (28), virulence gene sequencing, and in silico comparisons of the two O157 genomes (29, 30).

SEQ ID NOs. 1-82 include the nucleotide sequences for the genes or ORFs in which the 96 SNPs are located.

TABLE 4

| | | | | | | | | | | | | |
|---|---|---|---|---|---|---|---|---|---|---|---|---|
| | | | | SEQ | | | | | | Original | SNP | |
| | SNP | | | ID | SNP | Genome | Sakai | Test | | Amino | Amino | |
| SNP# | Label | Min.* | Gene | NO. | position | Location | SNP | SNP | Type† | Acid | Acid | Function |

| | | | | | | | | | | | | |
|---|---|---|---|---|---|---|---|---|---|---|---|---|
| 1 | 03_83 | 1 | ECs0333 | 1 | 83 | 351109 | T | C | N | V | A | putative |
| | | | | | | | | | | | | transcriptional |
| | | | | | | | | | | | | regulator |
| 2 | 05_429 | 0 | ECs0495 | 2 | 429 | 528395 | C | T | S | N | N | putative protease |
| | | | | | | | | | | | | maturation protein |
| 3 | 40_1060 | 0 | ECs2521 | 3 | 1060 | 2497693 | T | G | N | S | A | p-aminobenzoate |
| | | | | | | | | | | | | synthetase |
| | | | | | | | | | | | | component I |
| 4 | 95_739 | 1 | ECs2006 | 4 | 739 | 1984857 | G | A | N | D | N | putative BigA-like |
| | | | | | | | | | | | | protein |
| 5 | 07_219 | 0 | ECs0593 | 5 | 219 | 651644 | T | C | S | F | F | putative chaperone |
| 6 | 09_117 | 1 | ECs0606 | 6 | 117 | 673343 | A | G | N | E | D | hypothetical protein |
| 7 | 48_190 | 0 | ECs3022 | 7 | 190 | 2954379 | T | G | N | C | G | hypothetical protein |
| 8 | 49_1060 | 0 | ECs3027 | 8 | 1060 | 2959611 | C | A | S | R | R | putative salicylate |
| | | | | | | | | | | | | hydroxylase |
| 9 | 50_39 | 0 | ECs3044 | 9 | 39 | 2977922 | T | C | S | V | V | hypothetical protein |
| 10 | 12_1151 | 1 | ECs0625 | 10 | 1151 | 696963 | C | T | N | P | L | enterobactin |
| | | | | | | | | | | | | synthetase |
| | | | | | | | | | | | | component EntF |
| 11 | 13_125 | 1 | ECs0654 | 11 | 125 | 730801 | T | G | N | L | R | citrate lyase alpha |
| | | | | | | | | | | | | chain |
| 12 | 14_281 | 1 | ECs0654 | 11 | 281 | 730645 | T | G | N | I | T | citrate lyase alpha |
| | | | | | | | | | | | | chain |
| 13 | 51_1490 | 0 | ECs3099 | 12 | 1490 | 3038252 | A | G | N | K | R | putative |
| | | | | | | | | | | | | malate:quinone |
| | | | | | | | | | | | | oxidoreductase |
| 14 | 52_2237 | 0 | ECs3221 | 13 | 2237 | 3179215 | G | C | N | G | A | putative outer |
| | | | | | | | | | | | | membrane protein |
| 15 | 15_150 | 0 | ECs0655 | 14 | 150 | 731085 | G | C | N | E | D | citrate lyase beta |
| | | | | | | | | | | | | chain |
| 16 | 17_339 | 1 | ECs0712 | 15 | 339 | 789194 | T | C | S | D | D | hypothetical protein |
| 17 | 18_1678 | 1 | ECs0721 | 16 | 1678 | 797116 | G | C | N | V | L | ornithine |
| | | | | | | | | | | | | decarboxylase |
| | | | | | | | | | | | | isozyme |
| 18 | 04_1545 | 1 | ECs0472 | 17 | 1545 | 501564 | G | A | N | M | I | hypothetical protein |
| 19 | 58_379 | 1 | ECs3609 | 18 | 379 | 3599366 | C | T | N | P | S | hypothetical protein |
| 20 | 61_175 | 0 | ECs3788 | 19 | 175 | 3800637 | A | G | N | I | V | ATPase component |
| | | | | | | | | | | | | of arginine |
| | | | | | | | | | | | | trasnporter |
| 21 | 19_928 | 1 | ECs0915 | 20 | 928 | 1002396 | G | A | N | G | S | hypothetical protein |
| 22 | 20_311 | 1 | ECs0942 | 21 | 311 | 1027219 | A | G | N | E | G | hypothetical protein |
| 23 | 62_259 | 1 | ECs3830 | 22 | 259 | 3838445 | C | T | N | R | C | putative ribosomal |
| | | | | | | | | | | | | protein |
| 24 | 64_438 | 0 | ECs3881 | 23 | 438 | 3885057 | T | C | S | T | T | hydrogenase-2 |
| | | | | | | | | | | | | small subunit |
| 25 | 65_1909 | 0 | ECs3917 | 24 | 1909 | 3919301 | T | G | N | C | G | putative ferrichrome |
| | | | | | | | | | | | | iron receptor |
| | | | | | | | | | | | | precursor |
| 26 | 28_774 | 0 | ECs1272 | 25 | 774 | 1338134 | T | A | S | S | S | Rtn-like protein |
| 27 | 29_2064 | 0 | ECs1282 | 26 | 2064 | 1352003 | C | T | S | Y | Y | hemagglutinin/ |
| | | | | | | | | | | | | hemolysin-related |
| | | | | | | | | | | | | protein |
| 28 | 67_283 | 0 | ECs3972 | 27 | 283 | 3981094 | G | A | N | V | I | hypothetical protein |
| 29 | 68_2001 | 0 | ECs4022 | 28 | 2001 | 4032354 | G | A | S | T | T | putative outer |
| | | | | | | | | | | | | membrane protein |
| 30 | 69_630 | 0 | ECs4130 | 29 | 630 | 4143190 | T | C | S | G | G | sodium/pantothenate |
| | | | | | | | | | | | | symporter |
| 31 | 30_717 | 0 | ECs1496 | 30 | 717 | 1537161 | T | C | S | R | R | putative kinase |
| 32 | 84_441 | 0 | Ecs4834 | 31 | 441 | 4901210 | A | G | S | Q | Q | superoxide |
| | | | | | | | | | | | | dismutase SodA |
| 33 | 34_1368 | 0 | ECs2071 | 32 | 1368 | 2060459 | T | C | S | P | P | cryptic nitrate |
| | | | | | | | | | | | | reductase 2 alpha |
| | | | | | | | | | | | | subunit |
| 34 | 70_984 | 0 | ECs4251 | 33 | 984 | 4253565 | G | A | S | T | T | ferrous iron |
| | | | | | | | | | | | | transport protein B |
| 35 | 71_375 | 0 | ECs4305 | 34 | 375 | 4315671 | A | C | S | T | T | periplasmic binding |
| | | | | | | | | | | | | protein |
| 36 | 72_776 | 1 | ECs4380 | 35 | 776 | 4390671 | G | A | N | G | E | heme |
| | | | | | | | | | | | | utilization/transport |
| | | | | | | | | | | | | protein |
| 37 | 35_849 | 1 | ECs2082 | 36 | 849 | 2074263 | G | A | S | V | V | alcohol |
| | | | | | | | | | | | | dehydrogenase |
| 38 | 41_1612 | 0 | Ecs2598 | 37 | 1612 | 2575641 | C | T | N | R | C | sensory transducer |
| | | | | | | | | | | | | kinase CheA |
| 39 | 37_539 | 0 | ECs2357 | 38 | 539 | 2326287 | C | A | N | S | Y | hypothetical protein |
| 40 | 01_1425 | 0 | ECs0127 | 39 | 1425 | 142879 | C | A | S | V | V | hypothetical protein |
| 41 | 76_246 | 0 | ECs4479 | 40 | 246 | 4518729 | G | T | S | V | V | hypothetical protein |
| 42 | 78_295 | 0 | ECs4502 | 41 | 295 | 4546915 | C | T | S | L | L | putative |
| | | | | | | | | | | | | glucosyltransferase |
| 43 | 79_37 | 0 | ECs4589 | 42 | 37 | 4620815 | A | G | N | T | A | hypothetical protein |
| 44 | 82_1470 | 0 | ECs4667 | 43 | 1470 | 4701702 | C | T | S | G | G | putative outer |
| | | | | | | | | | | | | membrane usher |
| | | | | | | | | | | | | protein precursor |
| 45 | 83_1484 | 0 | ECs4820 | 44 | 1484 | 4882975 | A | C | N | E | G | formate |
| | | | | | | | | | | | | dehydrogenase-O |
| | | | | | | | | | | | | major subunit |
| 46 | fadD- | 0 | ECs2514 | 45 | 1198 | 2490378 | T | C | N | S | P | acyl coenzyme A |
| | 1198 | | | | | | | | | | | synthetase |
| 47 | 66_348 | 1 | ECs3942 | 46 | 348 | 3944571 | A | C | S | A | A | hypothetical protein |
| 48 | fimA-299 | 1 | ECs5273 | 47 | 299 | 5398304 | T | C | N | V | A | major type 1 subunit |
| | | | | | | | | | | | | fimbrin |
| 49 | 85_1340 | 1 | ECs4889 | 48 | 1340 | 4964826 | G | A | N | R | Q | argininosuccinate |
| | | | | | | | | | | | | lyase |
| 50 | 86_219 | 0 | ECs5009 | 49 | 219 | 5089398 | A | G | S | T | T | hypothetical protein |
| 51 | fimA-354 | 1 | ECs5273 | 47 | 354 | 5398359 | C | A | N | T | R | major type 1 subunit |
| | | | | | | | | | | | | fimbrin |
| 52 | fimA-468 | 0 | ECs5273 | 47 | 468 | 5398473 | C | T | S | F | F | major type 1 subunit |
| | | | | | | | | | | | | fimbrin |
| 53 | fimA-469 | 0 | ECs5273 | 47 | 469 | 5398474 | C | T | N | Q | Ter | major type 1 subunit |
| | | | | | | | | | | | | fimbrin |
| 54 | 90_1097 | 0 | ECs5206 | 50 | 1097 | 5307634 | G | A | N | R | Q | putative ATP- |
| | | | | | | | | | | | | binding component |
| | | | | | | | | | | | | of a transport |
| | | | | | | | | | | | | system |
| 55 | adhP-452 | 0 | ECs2082 | 36 | 452 | 2074660 | A | G | N | N | S | alcohol |
| | | | | | | | | | | | | dehydrogenase |
| 56 | fimA-527 | 1 | ECs5273 | 47 | 527 | 5398532 | C | T | N | T | I | major type 1 subunit |
| | | | | | | | | | | | | fimbrin |
| 57 | 63_494 | 0 | ECs3880 | 51 | 494 | 3884025 | A | G | N | H | R | probable |
| | | | | | | | | | | | | cytochrome Ni/Fe |
| | | | | | | | | | | | | component of |
| | | | | | | | | | | | | hydrogenase-2 |
| 58 | 43_3971 | 1 | ECs2775 | 52 | 3971 | 2717449 | G | T | N | G | V | putative factor |
| 59 | arcA-450 | 0 | ECs5359 | 53 | 450 | 5496655 | T | G | S | S | S | aerobic regulator |
| 60 | arcA-492 | 0 | ECs5359 | 53 | 492 | 5496613 | T | C | S | S | S | aerobic regulator |
| 61 | rpoS_562 | 0 | ECs3595 | 54 | 562 | 3587513 | A | C | N | T | I | RNA polymerase |
| | | | | | | | | | | | | sigma factor |
| 62 | 38_77 | 0 | ECs2375 | 55 | 77 | 2346918 | C | T | N | P | L | hypothetical protein |
| 63 | 22_205 | 0 | ECs1028 | 56 | 205 | 1133596 | C | A | N | R | S | hypothetical protein |
| 64 | aspC-132 | 1 | ECs1011 | 57 | 132 | 1115049 | G | T | S | P | P | aspartate |
| | | | | | | | | | | | | aminotransferase |
| 65 | aspC-267 | 1 | ECs1011 | 57 | 267 | 1114914 | G | A | S | L | L | aspartate |
| | | | | | | | | | | | | aminotransferase |
| 66 | 96_592 | 0 | ECs5022 | 58 | 592 | 5106168 | A | T | N | T | S | chorismate lyase |
| 67 | 42_579 | 0 | ECs2696 | 59 | 579 | 2653334 | C | A | S | V | V | putative methyl- |
| | | | | | | | | | | | | independent |
| | | | | | | | | | | | | mismatch repair |
| | | | | | | | | | | | | protein |
| 68 | 87_255 | 0 | ECs5069 | 60 | 255 | 5161881 | A | G | S | L | L | putative aldolase |
| 69 | 80_242 | 0 | ECs4610 | 61 | 242 | 4640773 | C | A | N | T | K | hypothetical protein |
| 70 | clpX-363 | 0 | ECs0492 | 62 | 363 | 523840 | C | T | S | T | T | ATP-dependent |
| | | | | | | | | | | | | protease ATPase |
| | | | | | | | | | | | | subunit |
| 71 | cyaA-528 | 0 | ECs4736 | 63 | 528 | 4785338 | C | T | S | S | S | adenylate cyclase |
| 72 | mdh-312 | 0 | ECs4109 | 64 | 312 | 4119194 | A | G | S | Q | Q | malate |
| | | | | | | | | | | | | dehydrogenase |
| 73 | mdh-694 | 0 | ECs4109 | 64 | 694 | 4118812 | G | A | N | A | T | malate |
| | | | | | | | | | | | | dehydrogenase |
| 74 | 81_388 | 0 | ECs4655 | 65 | 388 | 4690099 | A | G | N | N | D | hypothetical protein |
| 75 | eae-2707 | 1 | ECs4559 | 66 | 2707 | 4596556 | C | A | N | T | I | intimin adherence |
| | | | | | | | | | | | | protein |
| 76 | eae-2741 | 0 | ECs4559 | 66 | 2741 | 4596522 | C | T | N | R | S | intimin adherence |
| | | | | | | | | | | | | protein |
| 77 | 60_144 | 1 | ECs3743 | 67 | 144 | 3744736 | A | G | S | L | L | putative carbamoyl |
| | | | | | | | | | | | | transferase |
| 78 | nlp-220 | 0 | ECs4067 | 68 | 220 | 4077482 | C | A | N | P | T | regulatory factor of |
| | | | | | | | | | | | | maltose metabolism |
| 79 | rpoS-431 | 0 | ECs3595 | 54 | 431 | 3587643 | C | T | N | T | T | RNA polymerase |
| | | | | | | | | | | | | sigma factor |
| 80 | 74_507 | 0 | ECs4426 | 69 | 507 | 4452577 | A | C | S | V | V | putative fimbrial |
| | | | | | | | | | | | | protein precursor |
| 81 | espA-339 | 1 | ECs4556 | 70 | 339 | 4593379 | T | A | N | R | S | LEE pathogenicity |
| | | | | | | | | | | | | island secreted |
| | | | | | | | | | | | | protein |
| 82 | espA-370 | 0 | ECs4556 | 70 | 370 | 4593348 | C | A | N | D | E | LEE pathogenicity |
| | | | | | | | | | | | | island secreted |
| | | | | | | | | | | | | protein |
| 83 | rpoS-543 | 0 | ECs3595 | 54 | 543 | 3587532 | A | C | S | K | Q | RNA polymerase |
| | | | | | | | | | | | | sigma factor |
| 84 | 59_279 | 0 | ECs3635 | 71 | 279 | 3626293 | A | C | S | G | G | hypothetical |
| | | | | | | | | | | | | membrane protein |
| 85 | 55_942 | 0 | ECs3336 | 72 | 942 | 3311013 | A | G | S | L | L | hypothetical protein |
| 86 | uidA-686 | 0 | ECs2325 | 73 | 686.1 | 2295005 | GG | — | insert | | | interrupted beta-D- |
| | | | | | | | | | | | | glucuronidase |
| 87 | uidA-693 | 1 | ECs2324 | 74 | 693 | 2294999 | C | T | S | R | Q | interrupted beta-D- |
| | | | | | | | | | | | | glucuronidase |
| 88 | uidA-776 | 0 | ECs2325 | 73 | 776 | 2294916 | G | A | N | S | S | interrupted beta-D- |
| | | | | | | | | | | | | glucuronidase |
| 89 | yjdB- | 1 | ECs5096 | 75 | 1186 | 5188884 | C | G | N | R | G | hypothetical protein |
| | 1186 | | | | | | | | | | | |
| 90 | 26_510 | 0 | ECs1262 | 76 | 510 | 1322616 | T | C | S | A | A | hypothetical protein |
| 91 | yjfG-308 | 0 | ECs5210 | 77 | 308 | 5311573 | A | G | N | H | R | putative ligase |
| 92 | yjiM-417 | 1 | ECs5298 | 78 | 417 | 5428580 | T | C | S | S | S | hypothetical protein |
| 93 | 06_247 | 1 | ECs0517 | 79 | 247 | 552072 | A | G | N | S | G | acrAB operon |
| | | | | | | | | | | | | repressor |
| 94 | 32_561 | 0 | ECs1860 | 80 | 561 | 1850330 | G | A | S | V | V | putative |
| | | | | | | | | | | | | oxidoreductase |
| 95 | 33_2244 | 1 | ECs1895 | 81 | 2244 | 1887941 | T | C | S | A | A | hypothetical protein |
| 96 | 46_648 | 1 | ECs2852 | 82 | 648 | 2796191 | T | C | S | D | D | putative colanic acid |
| | | | | | | | | | | | | biosynthsis carrier |
| | | | | | | | | | | | | transferase |

Table 4 shows ninety-six single nucleotide polymorphism (SNP) loci examined by real time PCR assays. In the column identified as “Min” the number “1” is used to show the SNPs that are in both the initial set of 32 SNP loci and in the set of 96 SNP loci; and “0” is used only in 96 SNP loci set. “N” means non-synonymous substitution; and “S” means synonymous substitution.

Hairpin-shaped primers (Table 5) were designed by adding a 5′ tail complementary to the 3′ end of each linear primer (22) for each locus, and real-time PCR was used to identify the SNP. Six strains were duplicated to serve as internal controls; identical SNP profiles were observed. Table 5 shows the primers trios (three primers for each SNP of the 96 SNPs) used to detect the SNPs (See, SEQ ID NOs 83-382).

TABLE 5

| | | HAIRPIN | | SEQ |
|---|---|---|---|---|
| SECTION | LABEL | PRIMER-1 | PRIMER SEQUENCE | ID NO. |
| A1 | 01_1425A | N-01_1425C-RHP | CGAAGGCA GCACTTCACTGATATTGCCTTCG | 83 |
| A2 | 03_83T | 03_83T-FHP | ACGGCTTGGCAGTTTTTCCAAAGCCGT | 86 |
| A3 | 04_1545A | N-04_1545G-RHP | GAGCAATTGT CAGTCGACGAACTCATAACAATTGCTC | 89 |
| A4 | 05_429C | 05_429C-FHP | GTTGCGGCAGCTATAACGGTATCCGCAAC | 92 |
| A5 | 06_247A | 06_247A-FHP | TAGGGAACTGAGTATCAGGCAAAGTTCCCTA | 95 |
| A6 | 07_219T | 07_219T-FHP | AAATGCCTCAGCGGTGTAAAAGAAAAGGCATTT | 98 |
| A7 | 09_117A | 09_117A-RHP | ACCCGTGGTTGCCTGTGAAACGGGT | 101 |
| A8 | 12_1151C | 12_1151C-FHP | GGGACCAGCTTGAACTGGCCCTGGTCCC | 104 |
| A9 | 13_125T | 13_125T-FHP | AGCGCTTACCAGGCTGAAAAAGCGCT | 107 |
| A10 | 14_281T | 14_281T-FHP | ATCCGGTGAAGATGGGCTTTAAAAACCGGAT | 110 |
| A11 | 15_150G | 15_150G-RHP | GTCCGTGTTTCACCTAATGCCACGGAC | 113 |
| A12 | 17_339T | 17_339T-FHP | ATCAGCTTTGGTACGCGCGATAAAGCTGAT | 116 |
| A13 | 18_1678G | 18_1678G-RHP | GTACGCTTCAGCAGTTTTTCGAAGCGTAC | 119 |
| A14 | 19_928G | 19_928G-FHP | CAGGGCACTTTATTGTCGGCTGCCCTG | 122 |
| A15 | 20_311A | 20_311A-FHP | TCGCTGGGAAGATGGCAGCGA | 125 |
| A16 | 22_205A | 21_79T-FHP | AGCAACGTTCGCCCTTTTATCGTTGCT | 128 |
| A17 | 26_510T | 27_1325T-RHP | TCAGAGCATAACATGCAAACTTGTGCTCTGA | 131 |
| A18 | 28_774T | 28_774T-FHP | AGATATCCAGCTTATGGCAGCACTGGATATCT | 134 |
| A19 | 29_2064C | 29_2064C-RHP | CAACAACCACTCCAGGTGGTAGCGTGGTTGTTG | 137 |
| A20 | 30_717T | 30_717T-FHP | ACGTACCAACGCCAATAACCTGGTACGT | 140 |
| A21 | 32_561G | 32_561G-FHP | CACACAG TCTTACTGCCTGCGACTGTGTG | 143 |
| A22 | 33_2244T | 33_2244T-RHP | TACCACG TCATCCTCCTGATACGTGGTA | 146 |
| A23 | 34_1368T | 34_1368T-FHP | AGGTCATTGTGTCCTGGTGCGTCAATGACCT | 149 |
| A24 | 35_849G | 35_849G-FHP | CACAAGACGCCTAGATATCCCACGTCTTGTG | 152 |
| A25 | 37_539C | 37_539C-RHP | CCGAGCGTTTTCCAGTGGCTCGG | 155 |
| A26 | 38_77C | 38_77C-FHP | GGAGTTTGTTG TCGCTTCTACACCAACAAACTCC | 158 |
| A27 | 40_1060T | 40_1060T-FHP | AGTGTAACTGCGCAACTGCCAGAACAGTTACACT | 161 |
| A28 | 41_1612C | N-41_1612C-RHP | CGTGAAGC GGATGCAGAACGGCTTCACG | 164 |
| A29 | 42_579C | 42_579C-FHP | GACCAGAC GGGCGTCTACGGTCTGGTC | 167 |
| A30 | 43_3971G | 43_3971G-FHP | CCCGTG AAGTTACCTTTAAGGTCACGGG | 170 |
| A31 | 46_648T | 46_648T-FHP | ATCGCAC GCGATGCAAAGGTGCGAT | 173 |
| A32 | 48_190T | 48_190T-FHP | TGCGATGTTCAGGTTAGTGCCATCGCA | 176 |
| A33 | 49_1060C | 49_1060C-FHP | GCCCCAGACCCTTGAAATGGGGC | 179 |
| A34 | 50_39T | 50_39T-RHP | TGCCACCAGGATCCCCAGAGTGGCA | 182 |
| A35 | 51_1490A | 51_1490A-FHP | TTGCGTCGTTCCAGCTTATGGACGCAA | 185 |
| A36 | 52_2237G | 52_2237G-FHP | CCCTGCCAGTCCATGGTGCAGGG | 188 |
| A37 | 55_942A | 55_942A-FHP | TAGTTCAA CGCATTTACACCGTGTTGAACTA | 191 |
| A38 | 58_379C | 58_379C-RHP | CCACCGGCGAGCTAGCGGTGG | 194 |
| A39 | 59_279A | 59_279A-FHP | TCCATCATA GATAAAGACCGCTATGATGGA | 197 |
| A40 | 60_144A | 60_144A-FHP | TAGTGCTTT GCCGCAGAATTAAAAGCACTA | 200 |
| A41 | 61_175A | 61_175A-FHP | TGCCCACCCTACGACTGGGCA | 203 |
| A42 | 62_259C | 62_259C-FHP | GTGCGGGCCGGGTATTTACACCGCAC | 206 |
| A43 | 63_494A | 63_494A-FHP | TGCTGCA CTGGAAGGTGTCGCTGCAGCA | 209 |
| A44 | 64_438T | 64_438T-FHP | AGTGCACATTACGACTAAGACGTGTGCACT | 212 |
| A45 | 65_1909T | 65_1909T-RHP | TGCGTAACGAACGACGGGTTACGCA | 215 |
| A46 | 66_348A | 66_348A-FHP | TGCGATGA GCTTTTGGTACCATCGCA | 218 |
| A47 | 67_283G | 67_283G-FHP | CAGGCTGACGCGAAGTTCCATCAGCCTG | 221 |
| A48 | 68_2001G | 68_2001G-FHP | CGTCACACATCCATACTCATGGTGTGACG | 224 |
| A49 | 69_630T | 69_630T-RHP | TGGCTTAATCTGTACTGCGTTGATTAAGCCA | 227 |
| A50 | 70_984G | 70_984G-RHP | GCTCCACAGTCCAGGAAGTGGAGC | 230 |
| A51 | 71_375A | 71_375A-RHP | AAACCCTGTGGGTCAGCTCAGGGTTT | 233 |
| A52 | 72_776G | 72_776G-FHP | CCAACGGAAAATCAGCAGACCGTTGG | 236 |
| A53 | 74_507A | 74_507A-FHP | TACAAGGG GCACAGCGAATACCCTTGTA | 239 |
| A54 | 76_246G | 76_246G-FHP | CACTCGACGGCTTTAGAGGGTCGAGTG | 242 |
| A55 | 78_295C | 78_295C-FHP | GCGCCTCTGAGCTATTGAAGGCGC | 245 |
| A56 | 79_37A | 79_37A-FHP | TCCATATCCACTTTCACCGAATGGATATGGA | 248 |
| A57 | 80_242C | 80_242C-FHP | GTGCCTGT TCCACCCTATGACAGGCAC | 251 |
| A58 | 81_388A | 81_388A-FHPp | TCAGAAGC TTTATAGTGTAAGGCAAGAGCTTCTGA | 254 |
| A59 | 82_1470C | 82_1470C-FHP | GCCTTCGCAGCCGCATCGAAGGC | 257 |
| A60 | 83_1484A | 83_1484A-FHP | TCCTGGAGCTGCTGGAAGTCCAGGA | 260 |
| A61 | 84_441A | N-84_441A-RHP | AGACTCCA ACCCATCAGCGTGGAGTCT | 263 |
| A62 | 85_1340G | 85_1340G-RHP | GGGCGACTTACAAAAGCAATCGCCC | 266 |
| A63 | 86_219A | 86_219A-RHP | AACCACGTGGGTACTGGTCGTCGTGGTT | 269 |
| A64 | 87_255A | 87_255A-FHP | TAGTCCTT GGTGTTAAATCTCGATCAAGGACTA | 272 |
| A65 | 88_1186C | 88_1186C-FHP | GGTGGCTCACCATAGGCAGCCACC | 275 |
| A66 | 90_1097G | 90_1097G-FHP | CGGGCTCGCTCTCCAAGCCCG | 278 |
| A67 | 91_299T | 91_299T-RHP | TGATTGACGGTATGACCCGCGTCAATCA | 281 |
| A68 | 95_739A | 95_739G-FHP | CGTCGTAAC GGCATCACCTCGAGTTACGACG | 284 |
| A69 | 96_592A | 96_592A-FHPp | ACGTCAC TTTCCTCTTAGTACAACAGTGACGT | 287 |
| A70 | adhP-452G | adhP-452G-RHP | GCAGCATTCCGGCACAGGTAATGCTGC | 290 |
| A71 | arcA-450G | arcA-450G-FHP | CGAACGGTGGACATCAACAGCCGTTCG | 293 |
| A72 | arcA-492C | arcA-492C-RHP | CGAGTTCCCATGGCGCGGAACTCG | 296 |
| A73 | aspC-132T | aspC-132T-RHP | TGTACTGACGCTTTTTCACGCTGGTCAGTACA | 299 |
| A74 | aspC-267A | aspC-267A-RHP | AATCAATGACACGAGCACGTTTGTCATTGATT | 302 |
| A75 | citF-125G | citF-125G-RHP | GCGATCGGCCCACAGTTTGCGATCGC | 305 |
| A76 | clpX-363T | clpX-363T-RHP | TGGTTCCAGCGTTTTACCGGAACCA | 308 |
| A77 | cyaA-528T | cyaA-528T-RHP | TACCCAGAAGCACCAGTATATGCTGGGTA | 311 |
| A78 | eae-2707A | eae-2707A-RHP | AGTTCTGGATGTTATAAGTGCTTGATAATCCAGAACT | 314 |
| A79 | eae-2741T | eae-2741T-RHP | TACAAAACCGCCAGGAAGAGGGTTTTGTA | 317 |
| A80 | espA-339A | espA-339A-RHP | ACCACGTAACCAGTTACACTTATGTCATTACGTGGT | 320 |
| A81 | espA-370A | espA-370A-FHP | TAATACCAGTTACCACGTAATGACATAAGTGTAACTGGTATTA | 323 |
| A82 | fadD- | fadD-1198C-RHP | CCGCCCCTGGCTGACCTGGCGG | 326 |
| | 1198C | | | |
| A83 | fimA-299C | fimA-299C-FHP | GCCGTACGCTGTTGCCTTTTTAGGTACGGC | 329 |
| A84 | fimA- | fimA-354A-FHP | TCTACCCAGAGTTCAGCTGCGGGTAGA | 332 |
| | 354A | | | |
| A85 | fimA-468T | fimA-468T-FHP | AAACGGAAACGGTACTAACACCATTCCGTTT | 335 |
| A86 | fimA-469T | fimA-469T-RHP | TAGGCGGATTGCATAATAACGCGCCTA | 338 |
| A87 | fimA-527T | fimA-527T-FHP | ATCGCATCGCTGCTAATGCGGATGCGAT | 341 |
| A88 | hybA- | hybA-438C-FHP | GGTGCACAATTACGACAAAGACGTGTGCACC | 344 |
| | 438C | | | |
| A89 | mdh-312G | mdh-312G-FHP | CTGCTGTACGCGTGAAAAACCTGGTACAGCAG | 347 |
| A90 | mdh-694A | mdh-694A-RHP | ACACGTTTGAGACAGGCCAAAACGTGT | 350 |
| A91 | nlp-220A | nlp-220A-RHP | ACCCATGATTCTGTCGATAAACTCATGGGT | 353 |
| A92 | N- | N-rpoS_562A- | AAGCTGGA CACTTGGTTCATGCTCCAGCTT | 356 |
| | rpoS_562A | RHP | | |
| A93 | rpoS-431T | rpoS-431T-RHP | TATACGCAAGAATCCACCAGGTTGCGTATA | 359 |
| A94 | rpoS-543C | rpoS-543C-FHP | GGTTCGCTGAACGTTTACCTGCGAACC | 362 |
| A95 | uidA- | uidA-686CA-FHP | TGCCTTGGTTGCAACTGGACAAGGCA | 365 |
| | 686CA | | | |
| A96 | uidA-693T | uidA-693T-RHP | TGGGACTCACCACTTGCAAAGTCCCA | 368 |
| A97 | uidA-776G | uidA-776G-RHP | GGACAGAGTCGGGTAGATATCACACTCTGTCC | 371 |
| A98 | yjdB- | yjdB-1186G-RHP | GGTCCGCGGTTGTAATAGGTCGGACC | 374 |
| | 1186G | | | |
| A99 | yjfG-308G | yjfG-308G-RHP | GCTGGGAACGGCCAGCACCCAGC | 377 |
| A100 | yjiM-417C | yjiM-417C-FHP | GCTGTTTGTTGATGCAGCTGACAAACAGC | 380 |
| | | HAIRPIN | | SEQ |
| SECTION | LABEL | PRIMER-2 | PRIMER SEQUENCE | ID NO. |
| B1 | 01_1425A | N-01_1425A-RHP | AGAAGGCA GCACTTCACTGATATTGCCTTCT | 84 |
| B2 | 03_83T | 03_83-R | TCAGCTTGGTGTTAAGACGTTCC | 87 |
| B3 | 04_1545A | N-04_1545A-RHP | AAGCAATTGT CAGTCGACGAACTCATAACAATTGCTT | 90 |
| B4 | 05_429C | 05_429-R | CATAAAATCGGTACCAGCAACG | 93 |
| B5 | 06_247A | 06_247-R | GTCACCGTGGATTCAAGAACA | 96 |
| B6 | 07_219T | 07_219-R | TATTTTCGCTTTTGGGTTCACTAAC | 99 |
| B7 | 09_117A | 09_117-F | TCGCAATGGCAGGATCA | 102 |
| B8 | 12_1151C | 12_1151-R | GGATCTCAATACTCAAATCACCGTG | 105 |
| B9 | 13_125T | 13_125-R | ATGCCGTCCTGTAAACCAGA | 108 |
| B10 | 14_281T | 14_281-R | CGAATGTGTTCTACCAGCGG | 111 |
| B11 | 15_150G | 15_150-F | GCCGCAGCATGTTGTTTG | 114 |
| B12 | 17_339T | 17_339-R | GCAGCCAGGCGGTGC | 117 |
| B13 | 18_1678G | 18_1678-F | CTCCGGCAGAAGATATGGC | 120 |
| B14 | 19_928G | 19_928-R | AAGTCGAGTAGCATCTGGAAATCTT | 123 |
| B15 | 20_311A | 20_311-R | CCCACGAACTGTAGCGATTATG | 126 |
| B16 | 22_205A | 21_79-R | AATCGCGTTCCGCCG | 129 |
| B17 | 26_510T | 27_1325-F | CACCGTCTCTCTCCTTTCGATG | 132 |
| B18 | 28_774T | 28_774-R | TTCTTAATTTCTTCTGCCAGGGA | 135 |
| B19 | 29_2064C | 29_2064-F | TGACTCTGCAGGCGCAGAA | 138 |
| B20 | 30_717T | 30_717-R | TGGTCACTTCACCCGCATC | 141 |
| B21 | 32_561G | 32_561A-FHP | TACACAG TCTTACTGCCTGCGACTGTGTA | 144 |
| B22 | 33_2244T | 33_2244C-RHP | CACCACG TCATCCTCCTGATACGTGGTG | 147 |
| B23 | 34_1368T | 34_1368-R | TGCTGCCACCGGCTAATGT | 150 |
| B24 | 35_849G | 35_849-R | CGTGCCGACCAGCGA | 153 |
| B25 | 37_539C | 37_539-F | GAATCTGCAGGCCAAAATTTC | 156 |
| B26 | 38_77C | 38_77T-FHP | AGAGTTTGTTG TCGCTTCTACACCAACAAACTCT | 159 |
| B27 | 40_1060T | 40_1060-R | TTCGGAGCCCCGGTTATT | 162 |
| B28 | 41_1612C | N-41_1612T-RHP | TGTGAAGC GGATGCAGAACGGCTTCACA | 165 |
| B29 | 42_579C | 42_579A-FHP | TACCAGAC GGGCGTCTACGGTCTGGTA | 168 |
| B30 | 43_3971G | 43_3971T-FHP | ACCGTG AAGTTACCTTTAAGGTCACGGT | 171 |
| B31 | 46_648T | 46_648C-FHP | GTCGCAC GCGATGCAAAGGTGCGAC | 174 |
| B32 | 48_190T | 48_190-R | GCCTTCATTGGCACTACACAGAT | 177 |
| B33 | 49_1060C | 49_1060-R | TCTGCCTGCGATTTCCCT | 180 |
| B34 | 50_39T | 50_39-F | GCTCGACTTTGTTCGCGG | 183 |
| B35 | 51_1490A | 51_1490-R | TGCCGCTACATCACCGTTCA | 186 |
| B36 | 52_2237G | 52_2237-R | CCGAGAACTTACGGTAGCCA | 189 |
| B37 | 55_942A | 55_942G-FHP | CAGTTCAA CGCATTTACACCGTGTTGAACTG | 192 |
| B38 | 58_379C | 58_379-F | GTGCGCAAAATGTATGAATTACG | 195 |
| B39 | 59_279A | 59_279C-FHP | GCCATCATA GATAAAGACCGCTATGATGGC | 198 |
| B40 | 60_144A | 60_144G-FHP | CAGTGCTTT GCCGCAGAATTAAAAGCACTG | 201 |
| B41 | 61_175A | 61_175-R | TCCCTCTCGAATCAACAACATG | 204 |
| B42 | 62_259C | 62_259-R | GATTCTTTTGATCGGTCGCG | 207 |
| B43 | 63_494A | 63_494G-FHP | CGCTGCA CTGGAAGGTGTCGCTGCAGCG | 210 |
| B44 | 64_438T | 64_438-R | GGACAGGCGACCATGCAG | 213 |
| B45 | 65_1909T | 65_1909-F | GGCAATAACACACTGACGTTTGG | 216 |
| B46 | 66_348A | 66_348C-FHP | GGCGATGA GCTTTTGGTACCATCGCC | 219 |
| B47 | 67_283G | 67_283-R | CTGACAATCGTACCGATAACCG | 222 |
| B48 | 68_2001G | 68_2001-R | TCAGTAGCAATCCCCGGATA | 225 |
| B49 | 69_630T | 69_630-F | GGCACCGTTGTGCTGCTTAT | 228 |
| B50 | 70_984G | 70_984-F | CTATTTGTGCATGGTATTCAATGG | 231 |
| B51 | 71_375A | 71_375-F | GTGTTCTTCTTCTACCCAGCCTG | 234 |
| B52 | 72_776G | 72_776-R | TTTATAAGAAAGCTGCGCATCG | 237 |
| B53 | 74_507A | 74_507C-FHP | GACAAGGG GCACAGCGAATACCCTTGTC | 240 |
| B54 | 76_246G | 76_246-R | CCATTCTCTGTGGCGTCAAT | 243 |
| B55 | 78_295C | 78_295-R | AGAAAAATAATCAAATGAAAGCAAACG | 246 |
| B56 | 79_37A | 79_37-R | AATAGCTGAACAGTAACCGCGTTAG | 249 |
| B57 | 80_242C | 80_242A-FHP | TTGCCTGT TCCACCCTATGACAGGCAA | 252 |
| B58 | 81_388A | 81_388G-FHP | CCAGAAGC TTTATAGTGTAAGGCAAGAGCTTCTGG | 255 |
| B59 | 82_1470C | 82_1470-R | CGACTGAATGTTAAATAAATATTGCCC | 258 |
| B60 | 83_1484A | 83_1484-R | CGCTTTATCACCAAAGAAGGCC | 261 |
| B61 | 84_441A | N-84_441G-RHP | GGACTCCA ACCCATCAGCGTGGAGTCC | 264 |
| B62 | 85_1340G | 85_1340-F | GAAGATGTCTATCCGATTCTGTCG | 267 |
| B63 | 86_219A | 86_219-F | GTGTCGCGCTCGCGG | 270 |
| B64 | 87_255A | 87_255G-FHP | CAGTCCTT GGTGTTAAATCTCGATCAAGGACTG | 273 |
| B65 | 88_1186C | 88_1186-R | GTAAATTTCCTGAACTGCGGC | 276 |
| B66 | 90_1097G | 90_1097-R | GAAGGTGTGCGAATGCCAA | 279 |
| B67 | 91_299T | 91_1097-F | CTGGCACAGGACGGAGC | 282 |
| B68 | 95_739A | 95_739A-FHP | TGTCGTAAC GGCATCACCTCGAGTTACGACA | 285 |
| B69 | 96_592A | 96_592G-FHP | GCGTCAC TTTCCTCTTAGTACAACAGTGACGC | 288 |
| B70 | adhP-452G | adhP-452-F | ACGCGGTAAAAGTGCCAGA | 291 |
| B71 | arcA-450G | arcA-450-R | CAGCTTGTACTGCTCGCCA | 294 |
| B72 | arcA-492C | arcA-492-F | CCTGATGGCGAGCAGTACAA | 297 |
| B73 | aspC-132T | aspC-132-F | CCTCGGGA TTGGTGTCTATAAA | 300 |
| B74 | aspC-267A | aspC-267-F | AGGAACTGCTGTTTGGTAAAGGTA | 303 |
| B75 | citF-125G | citF-125-F | GATCTTGCCGCTTTCCAGA | 306 |
| B76 | clpX-363T | clpX-363-F | CGAGTTGGGCAAAAGTAACATTC | 309 |
| B77 | cyaA-528T | cyaA-528-F | GCCACAACGAGAGTGGCA | 312 |
| B78 | eae-2707A | eae-2707-F | CAATAACTGCTTGGATTAAACAGACA | 315 |
| B79 | eae-2741T | eae-2741-F | AGCAGCGTTCTGGAGTATCAAG | 318 |
| B80 | espA-339A | espA-339-F | AATGCGAAAGCCAAACTTCCT | 321 |
| B81 | espA-370A | espA-370-R | CACCAGCGCTTAAATCACCAC | 324 |
| B82 | fadD- | fadD-1198-F | TCATAGCGGTAGCATTGGTTTG | 327 |
| | 1198C | | | |
| B83 | fimA-299C | fimA-299-R | TCTGCAGAGCCAGAACGTTG | 330 |
| B84 | fimA- | fimA-354-R | CAGGATCTGCACACCAACGT | 333 |
| | 354A | | | |
| B85 | fimA-468T | fimA-468-R | CTCGCCGATTGCATAATAACG | 336 |
| B86 | fimA-469T | fimA-469-F | TGGTGCGACATTCAGTGAGC | 339 |
| B87 | fimA-527T | fimA-527-R | ATCCCTGCCCGTAATGACG | 342 |
| B88 | hybA- | hybA-438-R | GGCGACCATGCAGTAACG | 345 |
| | 438C | | | |
| B89 | mdh-312G | mdh-312-R | TGATAATACCAATGCACGCTTTC | 348 |
| B90 | mdh-694A | mdh-694-F | GGTCGGCAACCCTGTCTATG | 351 |
| B91 | nlp-220A | nlp-220-F | CCCTGGGTTATCTGGCCAT | 354 |
| B92 | N- | N-rpoS_562C- | CAGCTGGA CACTTGGTTCATGCTCCAGCTG | 357 |
| | rpoS_562A | RHP | | |
| B93 | rpoS-431T | rpoS-431-F | GGTAGAGAAGTTTGACCCGGAA | 360 |
| B94 | rpoS-543C | rpoS-543-R | GTCCAGCTTATGGGACAACTCA | 363 |
| B95 | uidA- | uidA-686-R | AGAGGTGCGGATTCACCACT | 366 |
| | 686CA | | | |
| B96 | uidA-693T | uidA-693-F | GAACTGCGTGATGCGGATC | 369 |
| B97 | uidA-776G | uidA-776-F | CGGGTGAAGGTTATCTCTATGAAC | 372 |
| B98 | yjdB- | yjdB-1186-F | GGTGATGGCGTGATTGTCTTA | 375 |
| | 1186G | | | |
| B99 | yjfG-308G | yjfG-308-F | CACGATTTTGTGCTGCGC | 378 |
| B100 | yjiM-417C | yjiM-417-R | TTTCCATAACGCACGCGAG | 381 |
| | | SHARED | | SEQ |
| SECTION | LABEL | PRIMER | PRIMER SEQUENCE | ID NO. |
| C1 | 01_1425A | N-01_1425-F | GCAAACCGCCAGCGGC | 85 |
| C2 | 03_83T | 03_83C-FHP | GCGGCTTGGCAGTTTTTCCAAAGCCGC | 88 |
| C3 | 04_1545A | N-04_1545-F | TGACCGAAACCATTGAGAATAATTTT | 91 |
| C4 | 05_429C | 05_429T-FHP | ATTGCGGCAGCTATAACGGTATCCGCAAT | 94 |
| C5 | 06_247A | 06_247G-FHP | CAGGGAACTGAGTATCAGGCAAAGTTCCCTG | 97 |
| C6 | 07_219T | 07_219C-FHP | GAATGCCTCAGCGGTGTAAAAGAAAAGGCATTC | 100 |
| C7 | 09_117A | 09_117C-RHP | CCCCGTGGTTGCCTGTGAAACGGGG | 103 |
| C8 | 12_1151C | 12_1151T-FHP | AGGACCAGCTTGAACTGGCCCTGGTCCT | 106 |
| C9 | 13_125T | 13_125G-FHP | CGCGCTTACCAGGCTGAAAAAGCGCG | 109 |
| C10 | 14_281T | 14_281C-FHP | GTCCGGTGAAGATGGGCTTTAAAAACCGGAC | 112 |
| C11 | 15_150G | 15_150C-RHP | CTCCGTGTTTCACCTAATGCCACGGAG | 115 |
| C12 | 17_339T | 17_339C-FHP | GTCAGCTTTGGTACGCGCGATAAAGCTGAC | 118 |
| C13 | 18_1678G | 18_1678C-RHP | CTACGCTTCAGCAGTTTTTCGAAGCGTAG | 121 |
| C14 | 19_928G | 19_928A-FHP | TAGGGCACTTTATTGTCGGCTGCCCTA | 124 |
| C15 | 20_311A | 20_311G-FHP | CCGCTGGGAAGATGGCAGCGG | 127 |
| C16 | 22_205A | 21_79C-FHP | GGCAACGTTCGCCCTTTTATCGTTGCC | 130 |
| C17 | 26_510T | 27_1325C-RHP | CCAGAGCATAACATGCAAACTTGTGCTCTGG | 133 |
| C18 | 28_774T | 28_774A-FHP | TGATATCCAGCTTATGGCAGCACTGGATATCA | 136 |
| C19 | 29_2064C | 29_2064T-RHP | TAACAACCACTCCAGGTGGTAGCGTGGTTGTTA | 139 |
| C20 | 30_717T | 30_717C-FHP | GCGTACCAACGCCAATAACCTGGTACGC | 142 |
| C21 | 32_561G | 32_561-R | GTACCGGATGCCCGAGATAA | 145 |
| C22 | 33_2244T | 33_2244-F | TATCCGTGGCTGAAGAATCTGTT | 148 |
| C23 | 34_1368T | 34_1368C-FHP | GGGTCATTGTGTCCTGGTGCGTCAATGACCC | 151 |
| C24 | 35_849G | 35_849A-FHP | TACAAGACGCCTAGATATCCCACGTCTTGTA | 154 |
| C25 | 37_539C | 37_539A-RHP | ACGAGCGTTTTCCAGTGGCTCGT | 157 |
| C26 | 38_77C | 38_77-R | CACTGTATGGCATCCCGACA | 160 |
| C27 | 40_1060T | 40_1060G-FHP | CGTGTAACTGCGCAACTGCCAGAACAGTTACACG | 163 |
| C28 | 41_1612C | N-41_1612-F | TTCATTCTGCCGCTGAATGC | 166 |
| C29 | 42_579C | 42_579-R | CCAGCCAATACCCCAGGT | 169 |
| C30 | 43_3971G | 43_3971-R | GACTATCTTCGTATCGTTGTTGCC | 172 |
| C31 | 46_648T | 46_648-R | CGAACAGGTGGTGTCCGC | 175 |
| C32 | 48_190T | 48_190G-FHP | GGCGATGTTCAGGTTAGTGCCATCGCC | 178 |
| C33 | 49_1060C | 49_1060A-FHP | TCCCCAGACCCTTGAAATGGGGA | 181 |
| C34 | 50_39T | 50_39C-RHP | CGCCACCAGGATCCCCAGAGTGGCG | 184 |
| C35 | 51_1490A | 51_1490G-FHP | CTGCGTCGTTCCAGCTTATGGACGCAG | 187 |
| C36 | 52_2237G | 52_2237C-FHP | GCCTGCCAGTCCATGGTGCAGGC | 190 |
| C37 | 55_942A | 55_942-R | AACCATTTTTTCCAGCGGG | 193 |
| C38 | 58_379C | 58_379T-RHP | TCACCGGCGAGCTAGCGGTGA | 196 |
| C39 | 59_279A | 59_279-R | TGATCCTGCCAGGCGACT | 199 |
| C40 | 60_144A | 60_144-R | TTGTCGCGGAATACGGAAAT | 202 |
| C41 | 61_175A | 61_175G-FHP | CGCCCACCCTACGACTGGGCG | 205 |
| C42 | 62_259C | 62_259T-FHP | ATGCGGGCCGGGTATTTACACCGCAT | 208 |
| C43 | 63_494A | 63_494-R | GCACCGAGCGCGATGA | 211 |
| C44 | 64_438T | 64_438C-FHP | GGTGCACATTACGACTAAGACGTGTGCACC | 214 |
| C45 | 65_1909T | 65_1909G-RHP | GGCGTAACGAACGACGGGTTACGCC | 217 |
| C46 | 66_348A | 66_348-R | AGTAACCAGGTTCCCGCCA | 220 |
| C47 | 67_283G | 67_283A-FHP | TAGGCTGACGCGAAGTTCCATCAGCCTA | 223 |
| C48 | 68_2001G | 68_2001A-FHP | TGTCACACATCCATACTCATGGTGTGACA | 226 |
| C49 | 69_630T | 69_630C-RHP | CGGCTTAATCTGTACTGCGTTGATTAAGCCG | 229 |
| C50 | 70_984G | 70_984A-RHP | ACTCCACAGTCCAGGAAGTGGAGT | 232 |
| C51 | 71_375A | 71_375C-RHP | CAACCCTGTGGGTCAGCTCAGGGTTG | 235 |
| C52 | 72_776G | 72_776A-FHP | TCAACGGAAAATCAGCAGACCGTTGA | 238 |
| C53 | 74_507A | 74_507-R | CAGGATGCTGGCCCAGTAACTT | 241 |
| C54 | 76_246G | 76_246T-FHP | AACTCGACGGCTTTAGAGGGTCGAGTT | 244 |
| C55 | 78_295C | 78_295T-FHP | ACGCCTCTGAGCTATTGAAGGCGT | 247 |
| C56 | 79_37A | 79_37G-FHP | CCCATATCCACTTTCACCGAATGGATATGGG | 250 |
| C57 | 80_242C | 80_37-R | TGCCGCCACCCAGGTA | 253 |
| C58 | 81_388A | 81_388-R | TATAAGAGAGAATCTCTCCATCATTTTTATAT | 256 |
| C59 | 82_1470C | 82_1470T-FHP | ACCTTCGCAGCCGCATCGAAGGT | 259 |
| C60 | 83_1484A | 83_1484G-FHP | CCCTGGAGCTGCTGGAAGTCCAGGG | 262 |
| C61 | 84_441A | N-84_441-F | CCCGCTTTGGTTCCGG | 265 |
| C62 | 85_1340G | 85_1340A-RHP | AGGCGACTTACAAAAGCAATCGCCT | 268 |
| C63 | 86_219A | 86_219G-RHP | GACCACGTGGGTACTGGTCGTCGTGGTC | 271 |
| C64 | 87_255A | 87_255-R | CTTGCACCACCGATTCAAAAT | 274 |
| C65 | 88_1186C | 88_1186G-FHP | CGTGGCTCACCATAGGCAGCCACG | 277 |
| C66 | 90_1097G | 90_1097A-FHP | TGGGCTCGCTCTCCAAGCCCA | 280 |
| C67 | 91_299T | 91_299C-RHP | CGATTGACGGTATGACCCGCGTCAATCG | 283 |
| C68 | 95_739A | 95_739-R | CTTTAGTGATGTGGATGAGTCCATCA | 286 |
| C69 | 96_592A | 96_592-R | AACCGCTGTTGCTAACAGAACTG | 289 |
| C70 | adhP-452G | adhP-452A-RHP | ACAGCATTCCGGCACAGGTAATGCTGT | 292 |
| C71 | arcA-450G | arcA-450T-FHP | AGAACGGTGGACATCAACAGCCGTTCT | 295 |
| C72 | arcA-492C | arcA-492T-RHP | TGAGTTCCCATGGCGCGGAACTCA | 298 |
| C73 | aspC-132T | aspC-132G-RHP | GGTACTGACGCTTTTTCACGCTGGTCAGTACC | 301 |
| C74 | aspC-267A | aspC-267G-RHP | GATCAATGACACGAGCACGTTTGTCATTGATC | 304 |
| C75 | citF-125G | citF-125T-RHP | TCGATCGGCCCACAGTTTGCGATCGA | 307 |
| C76 | clpX-363T | clpX-363C-RHP | CGGTTCCAGCGTTTTACCGGAACCG | 310 |
| C77 | cyaA-528T | cyaA-528C-RHP | CACCCAGAAGCACCAGTATATGCTGGGTG | 313 |
| C78 | eae-2707A | eae-2707C-RHP | CGTTCTGGATGTTATAAGTGCTTGATAATCCAGAACG | 316 |
| C79 | eae-2741T | eae-2741C-RHP | CACAAAACCGCCAGGAAGAGGGTTTTGTG | 319 |
| C80 | espA-339A | espA-339T-RHP | TCCACGTAACCAGTTACACTTATGTCATTACGTGGA | 322 |
| C81 | espA-370A | espA-370C-FHP | GAATACCAGTTACCACGTAATGACATAAGTGTAACTGGTATTC | 325 |
| C82 | fadD- | fadD-1198T-RHP | TCGCCCCTGGCTGACCTGGCGA | 328 |
| | 1198C | | | |
| C83 | fimA-299C | fimA-299T-FHP | ACCGTACGCTGTTGCCTTTTTAGGTACGGT | 331 |
| C84 | fimA- | fimA-354C-FHP | GCTACCCAGAGTTCAGCTGCGGGTAGC | 334 |
| | 354A | | | |
| C85 | fimA-468T | fimA-468C-FHP | GAACGGAAACGGTACTAACACCATTCCGTTC | 337 |
| C86 | fimA-469T | fimA-469C-RHP | CAGGCGGATTGCATAATAACGCGCCTG | 340 |
| C87 | fimA-527T | fimA-527C-FHP | GTCGCATCGCTGCTAATGCGGATGCGAC | 343 |
| C88 | hybA- | hybA-438T-FHP | AGTGCACAATTACGACAAAGACGTGTGCACT | 346 |
| | 438C | | | |
| C89 | mdh-312G | mdh-312A-FHP | TTGCTGTACGCGTGAAAAACCTGGTACAGCAA | 349 |
| C90 | mdh-694A | mdh-694G-RHP | GCACGTTTGAGACAGGCCAAAACGTGC | 352 |
| C91 | nlp-220A | nlp-220C-RHP | CCCCATGATTCTGTCGATAAACTCATGGGG | 355 |
| C92 | N- | N-rpoS_562-F | CCCGTACTATTCGTTTGCCGA | 358 |
| | rpoS_562A | | | |
| C93 | rpoS-431T | rpoS-431C-RHP | CATACGCAAGAATCCACCAGGTTGCGTATG | 361 |
| C94 | rpoS-543C | rpoS-543A-FHP | TGTTCGCTGAACGTTTACCTGCGAACA | 364 |
| C95 | uidA- | uidA-686iGG- | CCCCTTGGTTGCAACTGGACAAGGGG | 367 |
| | 686CA | FHP | | |
| C96 | uidA-693T | uidA-693C-RHP | CGGGACTCACCACTTGCAAAGTCCCG | 370 |
| C97 | uidA-776G | uidA-776A-RHP | AGACAGAGTCGGGTAGATATCACACTCTGTCT | 373 |
| C98 | yjdB- | yjdB-1186C-RHP | CGTCCGCGGTTGTAATAGGTCGGACG | 376 |
| | 1186G | | | |
| C99 | yjfG-308G | yjfG-308A-RHP | ACTGGGAACGGCCAGCACCCAGT | 379 |
| C100 | yjiM-417C | yjiM-417T-FHP | ACTGTTTGTTGATGCAGCTGACAAACAGT | 382 |

To reduce the number of SNP assays for classifying strains into SGs, the inventors used the SNPT program (21) that identified the initial set of 32 SNP loci (shown as “1” in the “Min” column of Table 4) to delineate 39 SGs. Additional assays were performed to confirm certain SGs. A second set of 32 SNP loci was developed which delineates 39 SGs. In this second set of 32 SNP loci as compared to the initial set of 32 SNP loci, three SNP loci that resolved SNP types 35 through 39 (fimA_{—}354, aspC_{—}267, and espA_{—}339) were substituted with three different loci for classifying SGs 1 through 34 (90_{—}1097G, espA_{—}370, and 26_{—}510).

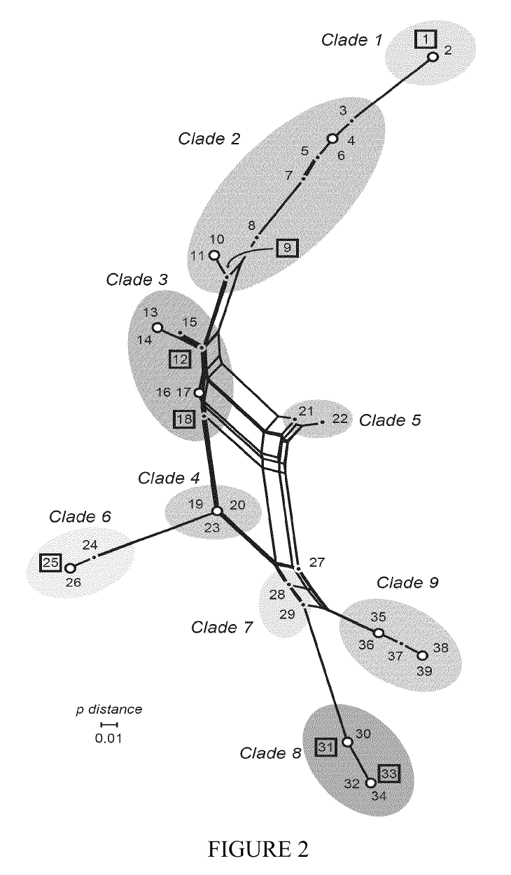

Those strains responsible for the extensive recombination depicted in FIG. 2 were submitted directly from a clinical laboratory and have since been found to be mixed O157 cultures. Therefore, the inventors identified a modified (third) set of 32 SNP loci that delineates 36 SGs; the 3 SGs generated because of O157 contamination were omitted. Specifically, this set does not include two SGs in clade 5 and SG-27. Table 6 shows the modified set of 32 SNP loci that can be used to delineate 36 SGs.

TABLE 6

seq ID No

| | | | | | | | | | | |
|---|---|---|---|---|---|---|---|---|---|---|
| 11 | 82 | 47 | 15 | 67 | 78 | 52 | 75 | 81 | 10 | 16 |

SNP #

| | SNP | 11 | 96 | 48 | 16 | 77 | 92 | 58 | 89 | 95 | 10 | 17 |
|---|---|---|---|---|---|---|---|---|---|---|---|---|
| clade | genotype(s) | 13_125 | 46_648 | fimA-299 | 17_339 | 60_144 | yjiM-417 | 43_3971 | yjdB-1186 | 33_2244 | 12-1151 | 18_1678 |
| 1 | 1 | T | T | T | T | A | T | G | C | T | C | G |
| 1 | 2 | G | T | T | T | A | T | G | C | T | C | G |
| 2 | 3 | G | C | T | T | A | T | G | C | T | C | G |
| 2 | 4, 6 | G | C | C | T | A | T | G | C | T | C | G |
| 2 | 5 | G | C | C | C | A | T | G | C | T | C | G |
| 2 | 7 | G | C | C | C | G | T | G | C | T | C | G |
| 2 | 8 | G | C | C | C | G | C | G | C | T | C | G |
| 2 | 9 | G | C | C | C | G | C | T | C | T | C | G |
| 2 | 10 | G | C | C | C | G | C | T | C | C | T | G |
| 2 | 11 | G | C | C | C | G | C | T | C | C | C | G |
| 3 | 12 | G | C | C | C | G | C | T | G | T | C | G |
| 3 | 13 | G | C | C | C | G | C | T | G | T | C | C |
| 3 | 14 | G | C | C | C | G | C | T | G | T | C | C |
| 3 | 15 | G | C | C | C | G | C | T | G | T | C | C |
| 3 | 16, 17 | G | C | C | C | G | C | T | G | T | C | G |
| 3 | 18 | G | C | C | C | G | C | T | G | T | C | G |
| 4 | 19 | G | C | C | C | G | C | T | G | T | C | G |
| 4, 5 | 20, 23 | G | C | C | C | G | C | T | G | T | C | G |
| 6 | 24 | G | C | C | C | G | C | T | G | T | C | G |
| 6 | 25 | G | C | C | C | G | C | T | G | T | C | G |
| 6 | 26 | G | C | C | C | G | C | T | G | T | C | G |
| 7 | 28 | G | C | C | C | G | C | T | G | T | C | G |
| 7 | 29 | G | C | C | C | G | C | T | G | T | C | G |
| 8 | 30 | G | C | C | C | G | C | T | G | T | C | G |
| 8 | 31 | G | C | C | C | G | C | T | G | T | C | G |
| 8 | 32 | G | C | C | C | G | C | T | G | T | C | G |
| 8 | 33 | G | C | C | C | G | C | T | G | T | C | G |
| 8 | 34 | G | C | C | C | G | C | T | G | T | C | G |
| 9 | 35 | G | C | C | C | G | C | T | G | T | C | G |
| 9 | 36 | G | C | C | C | G | C | T | G | T | C | G |
| 9 | 37 | G | C | C | C | G | C | T | G | T | C | G |
| 9 | 38 | G | C | C | C | G | C | T | G | T | C | G |
| 9 | 39 | G | C | C | C | G | C | T | G | T | C | G |

seq ID No

| | | | | | | | | | | | |
|---|---|---|---|---|---|---|---|---|---|---|---|
| 17 | 21 | 48 | 35 | 57 | 46 | 20 | 36 | 79 | 1 | 6 | 22 |

SNP #

| | SNP | 18 | 22 | 49 | 36 | 64 | 47 | 21 | 37 | 93 | 1 | 6 | 23 |
|---|---|---|---|---|---|---|---|---|---|---|---|---|---|
| clade | genotype(s) | 04_1545 | 20_311 | 85_1340 | 72_776 | aspC-132 | 66_348 | 19_928 | 35_849 | 06_247 | 03_83 | 09_117 | 62_259 |
| 1 | 1 | G | A | G | G | G | A | G | G | A | T | A | C |
| 1 | 2 | G | A | G | G | G | A | G | G | A | T | A | C |
| 2 | 3 | G | A | G | G | G | A | G | G | A | T | A | C |
| 2 | 4, 6 | G | A | G | G | G | A | G | G | A | T | A | C |
| 2 | 5 | G | A | G | G | G | A | G | G | A | T | A | C |
| 2 | 7 | G | A | G | G | G | A | G | G | A | T | A | C |
| 2 | 8 | G | A | G | G | G | A | G | G | A | T | A | C |
| 2 | 9 | G | A | G | G | G | A | G | G | A | T | A | C |
| 2 | 10 | G | A | G | G | G | A | G | G | A | T | A | C |
| 2 | 11 | G | A | G | G | G | A | G | G | A | T | A | C |
| 3 | 12 | G | A | G | G | G | A | G | G | A | T | A | C |
| 3 | 13 | G | A | G | G | G | C | G | G | A | T | A | C |
| 3 | 14 | A | A | G | G | G | A | G | G | A | T | A | C |
| 3 | 15 | G | A | G | G | G | A | G | G | A | T | A | C |
| 3 | 16, 17 | G | G | G | G | G | A | G | G | A | T | A | C |
| 3 | 18 | G | G | A | G | G | A | G | G | A | T | A | C |
| 4 | 19 | G | G | A | A | T | A | G | G | A | T | A | C |
| 4, 5 | 20, 23 | G | G | A | A | G | A | G | G | A | T | A | C |
| 6 | 24 | G | G | A | A | G | C | G | G | A | T | A | C |
| 6 | 25 | G | G | A | A | G | C | A | A | A | T | A | C |
| 6 | 26 | G | G | A | A | G | C | A | G | A | T | A | C |
| 7 | 28 | G | G | A | A | G | A | G | G | G | T | A | C |
| 7 | 29 | G | G | A | A | G | A | G | G | G | C | A | C |
| 8 | 30 | G | G | A | A | G | A | G | G | G | C | C | T |
| 8 | 31 | G | G | A | A | G | A | G | G | G | C | C | C |
| 8 | 32 | G | G | A | A | G | A | G | G | G | C | C | C |
| 8 | 33 | G | G | A | A | G | A | G | G | G | C | C | C |
| 8 | 34 | G | G | A | A | G | A | G | G | G | C | C | C |
| 9 | 35 | G | G | A | A | G | A | G | G | G | C | A | C |
| 9 | 36 | G | G | A | A | G | A | G | G | G | C | A | C |
| 9 | 37 | G | G | A | A | G | A | G | G | G | C | A | C |
| 9 | 38 | G | G | A | A | G | A | G | G | G | C | A | C |
| 9 | 39 | G | G | A | A | G | A | G | G | G | C | A | C |

seq ID No

| | | | | | | | | |
|---|---|---|---|---|---|---|---|---|
| 18 | 4 | 47 | 74 | 11 | 57 | 66 | 47 | 70 |

SNP #

| | SNP | 19 | 4 | 56 | 87 | 12 | 65 | 75 | 51 | 81 |
|---|---|---|---|---|---|---|---|---|---|---|
| clade | genotype(s) | 58_379 | 95_739 | fimA-527 | uidA-693 | 14_281 | aspC-267 | eae-2707 | fimA-354 | espA-339 |
| 1 | 1 | C | G | C | C | T | G | C | C | T |
| 1 | 2 | C | G | C | C | T | G | C | C | T |
| 2 | 3 | C | G | C | C | T | G | C | C | T |
| 2 | 4, 6 | C | G | C | C | T | G | C | C | T |
| 2 | 5 | C | G | C | C | T | G | C | C | T |
| 2 | 7 | C | G | C | C | T | G | C | C | T |
| 2 | 8 | C | G | C | C | T | G | C | C | T |
| 2 | 9 | C | G | C | C | T | G | C | C | T |
| 2 | 10 | C | G | C | C | T | G | C | C | T |
| 2 | 11 | C | G | C | C | T | G | C | C | T |
| 3 | 12 | C | G | C | C | T | G | C | C | T |
| 3 | 13 | C | G | C | C | T | G | C | C | T |
| 3 | 14 | C | G | C | C | T | G | C | C | T |
| 3 | 15 | C | G | C | C | T | G | C | C | T |
| 3 | 16, 17 | C | G | C | C | T | G | C | C | T |
| 3 | 18 | C | G | C | C | T | G | C | C | T |
| 4 | 19 | C | G | C | C | T | G | C | C | T |
| 4, 5 | 20, 23 | C | G | C | C | T | G | C | C | T |
| 6 | 24 | C | G | C | C | T | G | C | C | T |
| 6 | 25 | C | G | C | C | T | G | C | C | T |
| 6 | 26 | C | G | C | C | T | G | C | C | T |
| 7 | 28 | C | G | C | C | T | G | C | C | T |
| 7 | 29 | C | G | C | C | T | G | C | C | T |
| 8 | 30 | C | G | C | C | T | G | C | C | T |
| 8 | 31 | C | G | C | C | T | G | C | C | T |
| 8 | 32 | T | A | C | C | T | G | C | C | T |
| 8 | 33 | T | A | T | C | T | G | C | C | T |
| 8 | 34 | T | A | C | T | T | G | C | C | T |
| 9 | 35 | C | G | C | C | C | G | C | C | T |
| 9 | 36 | C | G | C | C | C | A | C | C | T |
| 9 | 37 | C | G | C | C | C | G | A | C | A |
| 9 | 38 | C | G | C | C | C | G | A | C | T |
| 9 | 39 | C | G | C | C | C | G | A | A | T |

The clade designations in Table 6 are shown, as follows: clade 1 is SG 1 and 2; clade 2 is SGs 3-11; clade 3 is SGs 12-18; clade 4 is SG 19 and 20; clade 5 is SG 23 (after the removal of SGs 21 and 22 which are mixed cultures); SG 23 is now classified as clade 5 because it is equidistant from SGs 20, 24, and 28; clade 6 is SGs 24-26; as compared to the original set, SG 27 was removed because of culture contamination; clade 7 is SG 28 and 29; clade 8 is SGs 30-34; and clade 9 is SGs 35-39. Three SGs (6, 17, and 23) cannot be distinguished from three other SGs using this particular system. Additional SNPs from Table 4 (96 loci) are required to differentiate these SGs.

Phylogenetic analyses. Distance between SGs was measured as the pairwise number of nucleotide difference. ME trees were used to infer the evolutionary relationships among the 39 SGs based on pairwise distance matrix with bootstrap replication for concatenated SNP data using MEGA3 (51). Bootstrap analysis of phylogenetic trees generated by the ME method were constructed using MEGA3 (51) and bootstrap confidence levels (based on 1000 replicate trees) were used to classify SGs into clades. A phylogenetic network based on the Neighbor-net algorithm (33) was applied to 48 PI sites using the SplitsTree4 program (52).

Spinach outbreak strain genomic analysis. A culture isolated from a Michigan patient hospitalized in September 2006, linked by the PulseNet PFGE system (53) to the spinach outbreak pattern by the MDCH and CDC, was sequenced. The Michigan State University (MSU) Genomic Research Support Technical Facility used parallel pyrosequencing on the GS20 454 that included four standard sequencing runs and one paired end run. The final assembly had 201 large contigs (>500 nt) with ˜20× coverage arranged into 79 scaffolds with a total of 5,307,096 nt, and 680 small contigs for a total of 213,699 nt (4% of the total assembled length). Contig alignments to published genomes (Sakai (29) and EDL-933 (30)) were conducted by MUMmer (38). Sakai/EDL-933 genes with at least one alignment of >90% nucleotide identity in the spinach genome were considered present in the spinach strain.

To evaluate the distribution of SNPs in the spinach genome, a strict set of comparison rules were applied. Conserved genes were included only if the alignment was 100% unique in both genomes (i.e., multi-copied genes in either genome were excluded), the identity between the aligned regions was over 90%, and the alignment region was more than 90% of the length of Sakai/EDL-933 genes. Insertions and deletions were excluded. A total of 2,741 genes that fit these criteria and occurred in all three genomes were compared to identify SNP differences. A map was plotted by GENOMEVIZ™ (54).

Stx2c detection. Multiplex PCR was used to detect stx2c and the Stx2c-phage o and q genes (39) in 519 strains; stx data was missing for 19 strains, 4 of which were repeatedly stx negative. The malate dehydrogenase (mdh) gene was used as a positive control. Strains were considered positive for stx2c if mdh (835 bp), stx2c (182 bp), o (533 bp), and q (321 bp) were present.

The multiplex PCR does not distinguish between stx2 and stx2c (both genes only differ by three amino acids in the B subunit (55)), thus the inventors developed a RFLP-based method that amplifies a larger PCR product (1152 bp) using primers stx2 F61 (5′-TATTCCCRGGARTTT AYGATAGA-3′) and stx2-2g_R1213 (5′-ATCCRGAGCCTGATKCAC AG-3′) (See, SEQ ID NOs. 383 and 384) PCR conditions include a 10-min soak at 94° C. and 35 cycles of: 92° C. for 1 min, 59° C. for 30 sec, 72° C. for 1 min, followed by a 5-min soak at 72° C. Digestion with FokI at 37° C. for 3 hours yields banding patterns specific for stx2 (453 bp, 362 bp, 211 bp, and 126 bp) or stx2c (488 bp, 453 by and 211 bp). All bands from each pattern are visible in strains with both stx2 and stx2c.

Epidemiological analyses. The inventors tested for differences in the frequency of clinical characteristics for Michigan patients using the Likelihood Chi Square test, and described the distributions using odds ratios with 95% confidence intervals. Clade 9 was omitted from the analysis as was one strain not part of a clade. To adjust for factors associated with infection by clade, we fit logistic regression models adjusting for age, gender and symptoms. The final epidemiologic analysis was limited to 333 of the 444 Michigan patients, as only one strain from each outbreak or cluster was included.

### Example 2

### SNP Genotyping and Diversity Among O157 Strains

A total of 96 SNP loci were evaluated in 83 O157 genes (FIG. 1A); 68 sites were identified by comparative genome microarrays (23), 15 from housekeeping genes (28), 4 by comparisons between two O157 genomes (29, 30), and 9 from three virulence genes (eae, espA, and fimA). Overall, 52 (54%) of the SNPs are non-synonymous and 43 (45%) are synonymous substitutions (FIG. 1A). One SNP locus detects a guanosine (G) dinucleotide insertion that results in a frameshift in the uidA gene and produces a premature termination codon. This uidA SNP (FIG. 1A) was examined because the GG insertion is hypothesized to have occurred late in the emergence of E. coli O157:H7 and its early origin explains the absence of beta-glucuronidase activity (i.e., GUD-phenotype) in most O157 strains (31).

Pairwise comparisons of the nucleotide profiles from 403 E. coli O157 and closely related strains from clinical sources worldwide distinguished 39 distinct SNP genotypes (SGs) (Table 3). Overall, the number of nucleotide differences between O157 SGs ranged from 1 to 57 with an average of 23.1±1.6 across the 96 loci. The nucleotide diversity, a measure of the degree of polymorphism within the O157 population, is 0.212±0.199, indicating that two strains selected at random differ on average at ˜20% of SNP loci (FIG. 1B). The minimum evolution (ME) algorithm, which infers that the theoretical tree is the smallest among all possible trees based on the sum of branch length estimates (32), revealed 9 clusters among the 39 genotypes (FIG. 1C). Eight of the nine clusters are significant (multiple SGs grouped with >85% bootstrap support). The deepest node in the ME phylogeny occurs at 15 SNP-locus differences and separates a lineage that includes ancestral O157 strains and close relatives with wildtype E. coli phenotypes (i.e., GUD+; sorbitol positive, Sor+) from the evolutionarily derived lineages (GUD−, Sor−) (FIG. 1C).

### Example 3

### Neighbor-Net Resolves Clades

Subsequent analyses of the 39 SG profiles revealed phylogenetically informative loci, as defined by two variants found in two or more SGs. Among the 96 SNP loci, 71 sites had complete data and, of these, there were 23 singletons and 48 parsimoniously informative (PI) sites. The 48 PI sites were used to construct a Neighbor-net tree (33) to determine if the informative sites support conflicting phylogenies or a single tree (FIG. 2). In this analysis, the 39 SGs were resolved into 25 distinct nodes: 10 nodes contained two or more SGs with the same profiles across all 48 loci (FIG. 2). Clade 9 roots the phylogenetic network because it includes strains with wildtype E. coli phenotypes (e.g. GUD+, Sor+), characteristics of the lineage most primitive to the derived EHEC O157 lineages (e.g. GUD−, Sor−) (31, 34). Rather than producing a unique bifurcating tree, the Neighbor-net reveals a central group of four clades (clade 3, 4, 5, and 7) connected by multiple paths. The presence of these parallel paths suggests that either recombination or recurrent mutation has contributed to the divergence of the central clades from the evolutionarily derived lineages. In contrast, clades 1, 2, 6, and 8 occur at the end of distinct branches with no evidence of conflicting phylogenetic signals, indicating that these lineages are diverging without evidence of recombination in background polymorphisms.

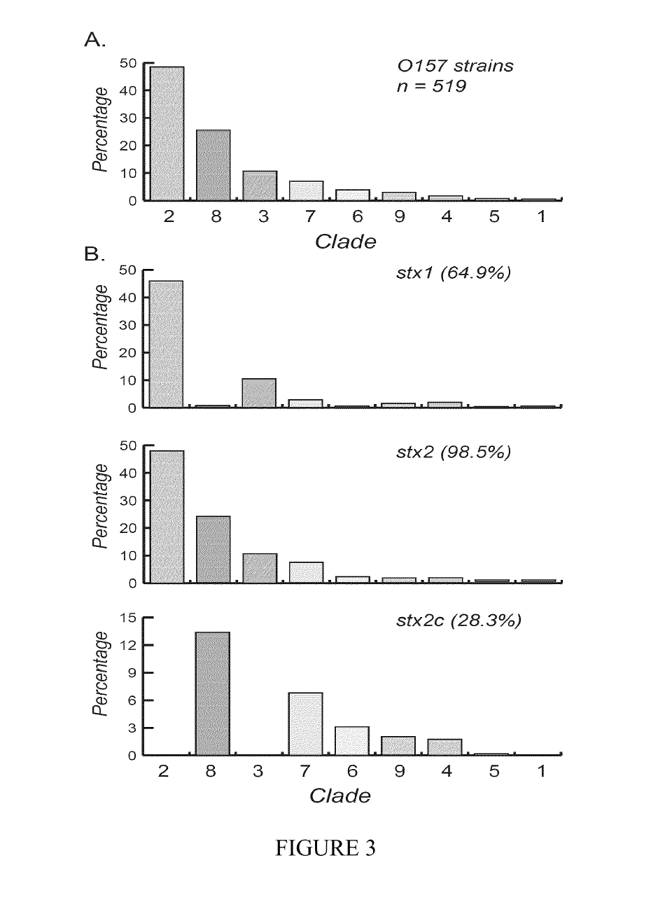

To further examine the distribution of O157 genotypes, the inventors devised a minimum set of 32 SNP loci for resolving all 39 SGs, and genotyped 135 additional O157 strains representing clinical sources, including five from well known outbreaks. In all, with the additional screening based on the minimal SNP set, 528 O157 strains were genotyped and classified into SGs and clades. Virtually all of the 528 strains were classified into one of 9 clades, and more than 75% of strains belonged to one of four clades. The most common genotypes were SG-9 (n=184; 35%) of clade 2 followed by SG-30 (n=94; 18%) of clade 8; 20 of the 39 SGs were only represented by one or two strains (FIG. 3A, Table 3). In addition, seven SGs were found among O157 strains isolated from multiple continents and during different time periods (Table 3). Five of these seven SGs belonged to the four clades located at the end of long branches identified in the Neighbor-net analysis (FIG. 2) and may represent stable EHEC O157 lineages generated from the central clades. Strains N0436 (SG-15), N0303 (SG-11), and N0587 (SG-27), which were included in a prior study of O157 SNPs (23) because they had uncommon PFGE patterns via PulseNet, represented unique, single strain SGs in this study as well. These SGs do not match other genotypes including SG-11 (N0303), which matches SG-10 at all 48 PI SNP loci.

### Example 4

### Shiga Toxin Genes in Clades

Because the production of Stx has been linked to virulence in O157 strains (35), we estimated the frequency of one or more of three Stx variants (stx1, stx2, and stx2c) by clade. Although stx1 was found in over half (˜65%) of 519 of the 528 O157 strains tested, the distribution is highly non-random across clades (FIG. 3B). The stx1 gene was common in clade 2 strains (95.1% of all stx1-positive strains are in clade 2) but not clade 8 (3.7%). The stx2 gene was present in virtually all (98.5%) O157 strains evaluated (FIG. 3B), occurring most frequently in clade 2 (46.8% of 519 strains) and clade 8 (25.4%) strains. In total, 98.4% and 100% of clade 2 and clade 8 strains, respectively, were positive for stx2 (FIG. 3B).

The stx2c gene also has a non-random distribution and is concentrated in clades 4, 6, 7, and 8 (FIG. 3B), but is missing from clades 1, 2, and 3. Most noteworthy is that clade 8 strains were significantly more likely to have both the stx2 and stx2c genes when compared to the other stx2c-positive clades (P<0.0001); 69 of the 79 O157 strains positive for both the stx2 and stx2c genes belonged to clade 8, but not all (57.6%) of the 128 clade 8 strains had stx2c.

### Example 5

### Virulence Differences Between O157 Clades

Clade 1 contains two SGs and includes the O157 genome strain, Sakai (29) (SG-1), implicated in the 1996 Japanese outbreak (Table 1) linked to radish sprouts (13). Clade 2, the predominant lineage identified, contains nine SGs and includes strain 93-111 (SG-9) from the 1993 outbreak associated with contaminated hamburgers in western North America (4). Clade 3 consists of seven genotypes and includes the genome strain EDL-933 (30) (SG-12) from the first human O157 outbreak in 1982 linked to hamburgers sold at a chain of fast food restaurant outlets in Michigan and Oregon (36). Although these outbreaks representing clades 1, 2, and 3 affected 12,000 people combined, the rate of HUS and hospitalization was low for each (4, 14, 15, 36) compared to the average rates for 350 North American outbreaks (3) (Table 1). Clade 8, in contrast, consists of five SGs that include O157 strains from multistate outbreaks linked to contaminated spinach (37) and lettuce (7) (SG-30) in North America. These 2006 outbreaks caused reportable illnesses in more than 275 patients and resulted in remarkably high rates of more severe disease, characterized by hospitalization (average 63%) and HUS (average 13%), a rate that is 3 times greater than the average HUS rate for 350 outbreaks (Table 1).

### Example 6

### Genome Sequencing of a Clade 8 Outbreak Strain

To assess whether the high rates of severe disease associated with the spinach outbreak are attributable to intrinsic differences between the spinach outbreak strain (clade 8) and other previously sequenced strains (e.g., Sakai, clade 1; EDL-933, clade 3), we used massively parallel pyrosequencing (GS 20, 454 Life Sciences, Branford, Conn.) to sequence the genome of a strain (TW14359) linked to the 2006 spinach outbreak. Contig alignment of the spinach outbreak strain to the O157 Sakai genome (29) using MUMmer (38) revealed 5,061 (96.3%) significant matches to the 5,253 Sakai genes. The spinach strain genome was missing 192 Sakai genes, 26 of which are backbone genes and 166 are genes for prophage and prophage-like elements. For example, the Mu-like phage Sp18 that is integrated into the sorbose operon of the Sakai genome (25) is absent in the spinach strain genome. Alignment to the Sakai pO157 plasmid revealed that 111 of 112 pO157 genes are present in the spinach outbreak strain, suggesting that the plasmid is conserved in both pathogens.

Among the 4,103 shared backbone genes within the Sakai and spinach genomes, the average sequence identity is 99.8%, and of the 958 shared island genes with Sakai, the average sequence identity is 97.96%. The average sequence identity for all shared genes (n=5,061) is 99.25%. We then compared the conservation of backbone genes and identified 2,741 shared genes with less than 0.5% nucleotide divergence among all three O157 genomes (FIG. 5). Interestingly, the Sakai and EDL-933 genomes are more similar to each other in gene content and nucleotide sequence identity than to the clade 8 spinach outbreak strain, which carries additional genetic material including stx2c and the Stx2c lysogenic bacteriophage 2851 (39). This suggests that the spinach outbreak genome, and by inference, clade 8, has substantial time to diverge with respect to its genetic composition when compared to strains from other lineages.

### Example 7

### Association Between Clades and Severe Disease

To determine if the O157 infections caused by clade 8 pathogens differ with respect to clinical presentation, the inventors examined epidemiological data for all laboratory-confirmed O157 cases (n=333 patients) identified in Michigan since 2001 (40). There are significant associations between specific O157 clades and patient symptoms as well as disease severity via univariate (Table 2) and multivariate (Table 7) analyses. Table 7 shows logistic regression results identifying predictors of hemolytic uremic syndrome (HUS) and infection with various E. coli O157 clades among 333 Michigan patients. *—the models used those without HUS as the reference group and were adjusted for bloody diarrhea, abdominal pain, diarrhea, chills, body aches, hospitalization, age and gender. †—the models used those infected with all other clades except clade 9 as the reference group and were adjusted for bloody diarrhea, abdominal pain, diarrhea, chills, body aches, HUS, hospitalization, age and gender.

TABLE 7
Logistic regression results identifying predictors of hemolytic uremic syndrome (HUS) and
infection with various Escherichia coli O157 clades among 333 Michigan patients.

| | HUS* | | Clade 8† | | Clade 2† | | Clade 7† | |
|---|---|---|---|---|---|---|---|---|
| Predictors | OR (95% CI) | P | OR (95% CI) | P | OR (95% CI) | P | OR (95% CI) | P |
| Bloody diarrhea | 0.8 (0.08, 8.50) | .88 | 1.5 (0.63, 3.51) | .36 | 1.6 (0.40, 1.14) | .15 | 0.1 (0.06, 0.35) | <.0001 |
| Abdominal pain | 0.4 (0.07, 1.85) | .22 | 2.0 (0.79, 5.07) | .14 | 1.2 (0.62, 2.23) | .61 | 0.5 (0.19, 1.28) | .15 |
| HUS | — | — | 7.0 (1.58, 31.31) | .01 | 0.5 (0.11, 1.92) | .29 | NA | .13 |
| Chills | 2.6 (0.37, 19.07) | .33 | 2.0 (0.94, 4.32) | .07 | 0.7 (0.38, 1.40) | .34 | 1.6 (0.55, 4.77) | .39 |
| Hospitalization | 4.7 (0.79, 27.65) | .09 | 1.5 (0.79, 2.74) | .23 | 0.9 (0.55, 1.49) | .70 | 1.1 (0.48, 2.64) | .78 |
| Age (0-18 years) | 16.70 (1.61, 172.78) | .02 | 2.0 (1.04, 3.82) | .04 | 0.7 (0.40, 1.14) | .15 | 1.0 (0.42, 2.34) | .97 |
| Female | 1.1 (0.25, 4.60) | .93 | 1.2 (0.64, 2.16) | .60 | 0.6 (0.34, 0.92) | .02 | 1.9 (0.77, 4.44) | .17 |
| Clade 8 infection | 6.1 (1.25, 29.94) | .03 | — | — | — | — | — | — |
| Clade 2 infection | 0.5 (0.11, 2.32) | .38 | — | — | — | — | — | — |

Patients infected with O157 strains of clade 8 were significantly more likely to be younger (ages 0 to 18), and despite the small number (n=11) of HUS cases identified, HUS patients were 7 times more likely to be infected with clade 8 strains than patients with strains from clades 1 to 7 combined (FIG. 4). This HUS association could not be explained by the presence of stx2c in clade 8 strains, as only 4 of 11 HUS patients had stx2c positive strains.

Three HUS patients had infections caused by strains of clade 2, the most numerically dominant clade, however, patients with HUS were still more likely to have a clade 8 infection when compared to clade 2 (Tables 2 and 7). In this analysis, the inventors also observed that clade 2 strains were more common in male patients, and clade 7 strains caused less severe disease, as measured by reporting frequencies of bloody diarrhea and other symptoms, though not all were significant (FIG. 4, Tables 2 and 7).

### Example 8

### Clade Frequencies Over Time

Because both the 2006 spinach and lettuce outbreaks were caused by members of the same SG within clade 8, the inventors estimated the frequency of clade 8 over time in an epidemiologically relevant setting. There was a significant increase (Mantel-Haenszel Chi Square=32.5, df=1, P<0.0001) in the frequency of disease caused by clade 8 strains among all 444 O157 cases in Michigan (Fig. S2). Specifically, the frequency of clade 8 strains increased from 10% in 2002 to 46% in 2006 despite the steady decrease in all O157 cases identified via surveillance (40) since 2002 (FIG. 6).

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

### REFERENCES

1. Caprioli, A., Morabito, S., Brugere, H. & Oswald, E. (2005) Vet Res 36, 289-311.
2. Mainil, J. G. & Daube, G. (2005) J Appl Microbiol 98, 1332-44.
3. Rangel, J. M., Sparling, P. H., Crowe, C., Griffin, P. M. & Swerdlow, D. L. (2005) Emerg Infect Dis 11, 603-9.
4. CDC (1993) Morb Mortal Wkly Rep 42, 258-63.
5. CDC (1995) Morb Mortal Wkly Rep 44, 157-60.
6. Hilborn, E. D., Mermin, J. H., Mshar, P. A., Hadler, J. L., Voetsch, A., Wojtkunski, C., Swartz, M., Mshar, R., Lambert-Fair, M. A., Farrar, J. A., Glynn, M. K. & Slutsker, L. (1999) Arch Intern Med 159, 1758-64.
7. CDC (2006) WEBSITE.
8. CDC (2006) Morb Mortal Wkly Rep 55, 1045-6.
9. Mead, P. S., Slutsker, L., Dietz, V., McCaig, L. F., Bresee, J. S., Shapiro, C., Griffin, P. M. & Tauxe, R. V. (1999) Emerg Infect Dis 5, 607-25.
10. Mead, P. S. & Griffin, P. M. (1998) Lancet 352, 1207-12.
11. Tan, P. I., Gordon, C. A. & Chandler, W. L. (2005) Lancet 365, 1073-86.
12. Reiss, G., Kunz, P., Koin, D. & Keeffe, E. B. (2006) J Am Geriatr Soc 54, 680-4.
13. Michino, H., Araki, K., Minami, S., Takaya, S., Sakai, N., Miyazaki, M., Ono, A. & Yanagawa, H. (1999) Am J Epidemiol 150, 787-96.
14. Fukushima, H., Hashizume, T., Morita, Y., Tanaka, J., Azuma, K., Mizumoto, Y., Kaneno, M., Matsuura, M., Konma, K. & Kitani, T. (1999) Pediatr Int 41, 213-7.
15. Higami, S., Nishimoto, K., Kawamura, T., Tsuruhara, T., Isshiki, G. & Ookita, A. (1998) Kansenshogaku Zasshi 72, 266-72.
16. Ostroff, S. M., Tarr, P. I., Neill, M. A., Lewis, J. H., Hargrett-Bean, N. & Kobayashi, J. M. (1989) J Infect Dis 160, 994-8.
17. Boerlin, P., McEwen, S. A., Boerlin-Petzold, F., Wilson, J. B., Johnson, R. P. & Gyles, C. L. (1999) J Clin Microbiol 37, 497-503.
18. Jelacic, J. K., Damrow, T., Chen, G. S., Jelacic, S., Bielaszewska, M., Ciol, M., Carvalho, H. M., Melton-Celsa, A. R., O'Brien, A. D. & Tarr, P. I. (2003) J infect Dis 188, 719-29.
19. Persson, S., Olsen, K. E., Ethelberg, S. & Scheutz, F. (2007) J Clin Microbiol 45, 2020-4.
20. Alland, D., Whittam, T. S., Murray, M. B., Cave, M. D., Hazbon, M. H., Dix, K., Kokoris, M., Duesterhoeft, A., Eisen, J. A., Fraser, C. M. & Fleischmann, R. D. (2003) J Bacteriol 185, 3392-9.
21. Filliol, I., Motiwala, A. S., Cavatore, M., Qi, W., Hernando Hazbon, M., Bobadilla Del Valle, M., Fyfe, J., Garcia-Garcia, L., Rastogi, N., Sola, C., Zozio, T., Guerrero, M. I., Leon, C. I., Crabtree, J., Angiuoli, S., Eisenach, K. D., Durmaz, R., Joloba, M. L., Rendon, A., Sifuentes-Osornio, J., Ponce de Leon, A., Cave, M. D., Fleischmann, R., Whittam, T. S. & Alland, D. (2006) J Bacteriol 188, 759-72.
22. Hazbon, M. H. & Alland, D. (2004) J Clin Microbiol 42, 1236-42.
23. Zhang, W., Qi, W., Albert, T. J., Motiwala, A. S., Alland, D., Hyytia-Trees, E. K., Ribot, E. M., Fields, P. I., Whittam, T. S. & Swaminathan, B. (2006) Genome Res 16, 757-67.
24. Kudva, I. T., Evans, P. S., Perna, N. T., Barrett, T. J., Ausubel, F. M., Blattner, F. R. & Calderwood, S. B. (2002) J Bacteriol 184, 1873-1879.
25. Ohnishi, M., Terajima, J., Kurokawa, K., Nakayama, K., Murata, T., Tamura, K., Ogura, Y., Watanabe, H. & Hayashi, T. (2002) Proc Natl Acad Sci USA 99, 17043-8.
26. Noller, A. C., McEllistrem, M. C., Stine, O. C., Morris, J. G., Jr., Boxrud, D. J., Dixon, B. & Harrison, L. H. (2003) J Clin Microbiol 41, 675-9.
27. Pearson, T., Busch, J. D., Ravel, J., Read, T. D., Rhoton, S. D., U'Ren, J. M., Simonson, T. S., Kachur, S. M., Leadem, R. R., Cardon, M. L., Van Ert, M. N., Huynh, L. Y., Fraser, C. M. & Keim, P. (2004) Proc Natl Acad Sci USA 101, 13536-41.
28. Hyma, K. E., Lacher, D. W., Nelson, A. M., Bumbaugh, A. C., Janda, J. M., Strockbine, N. A., Young, V. B. & Whittam, T. S. (2005) J Bacteriol 187, 619-28.
29. Hayashi, T., Makino, K., Ohnishi, M., Kurokawa, K., Ishii, K., Yokoyama, K., Han, C. G., Ohtsubo, E., Nakayama, K., Murata, T., Tanaka, M., Tobe, T., Iida, T., Takami, H., Honda, T., Sasakawa, C., Ogasawara, N., Yasunaga, T., Kuhara, S., Shiba, T., Hattori, M. & Shinagawa, H. (2001) DNA Research 8, 11-22.
30. Perna, N. T., Plunkett, G., Burland, V., Mau, B., Glasner, J. D., Rose, D. J., Mayhew, G. F., Evans, P. S., Gregor, J., Kirkpatrick, H. A., Posfai, G., Hackett, J., Klink, S., Boutin, A., Shao, Y., Miller, L., Grotbeck, E. J., Davis, N. W., Lim, A., Dimalanta, E. T., Potamousis, K. D., Apodaca, J., Anantharaman, T. S., Lin, J., Yen, G., Schwartz, D. C., Welch, R. A. & Blattner, F. R. (2001) Nature 409, 529-533.
31. Monday, S. R., Whittam, T. S. & Feng, P. C. (2001) J Infect Dis 184, 918-21.
32. Rzhetsky, A. & Nei, M. (1993) Mol Biol Evol 10, 1073-95.
33. Bryant, D. & Moulton, V. (2004) Mol Biol Evol 21, 255-65.
34. Feng, P., Lampel, K. A., Karch, H. & Whittam, T. S. (1998) J infect Dis 177, 1750-1753.
35. Paton, J. C. & Paton, A. W. (2003) Methods Mol Med 73, 9-26.
36. Riley, L. W., Remis, R. S., Helgerson, S. D., McGee, H. B., Wells, J. G., Davis, B. R., Hebert, R. J., Olcott, E. S., Johnson, L. M., Hargrett, N. T., Blake, P. A. & Cohen, M. L. (1983) N Engl J Med 308, 681-685.
37. FDA (2006) WEBSITE
38. Delcher, A. L., Phillippy, A., Carlton, J. & Salzberg, S. L. (2002) Nucleic Acids Res 30, 2478-83.
39. Strauch, E., Schaudinn, C. & Beutin, L. (2004) Infect Immun 72, 7030-9.
40. Manning, S. D., Madera, R. T., Schneider, W., Dietrich, S. E., Khalife, W., W. Brown, Whittam, T. S., Somsel, P. & Rudrik., J. T. (2006) Emerg Infect Dis 13, 318-321.
41. Robins-Browne, R. M. (2005) Clin Infect Dis 41, 793-794.
42. Kim, J., Nietfeldt, J. & Benson, A. K. (1999) Proc Natl Acad Sci USA 96, 13288-13293.
43. Noller, A. C., McEllistrem, M. C., Pacheco, A. G., Boxrud, D. J. & Harrison, L. H. (2003) J Clin Microbiol 41, 5389-97.
44. Shaikh, N. & Tarr, P. I. (2003) J Bacteriol 185, 3596-605.
45. CDC (2006) Morb Mortal Wkly Rep 55, 392-5.
46. Schmidt, H. (2001) Res Microbiol 152, 687-95.
47. Kaper, J. B., Nataro, J. P. & Mobley, H. L. (2004) Nat Rev Microbiol 2, 123-40.
48. Besser, T. E., Shaikh, N., Holt, N. J., Tarr, P. I., Konkel, M. E., Malik-Kale, P., Walsh, C. W., Whittam, T. S. & Bono, J. L. (2007) Appl Environ Microbiol 73, 671-9.
49. Steele, M., Ziebell, K., Zhang, Y., Benson, A., Konczy, P., Johnson, R. & Gannon, V. (2007) Appl Environ Microbiol 73, 22-31.
50. Kim, J., Nietfeldt, J., Ju, J., Wise, J., Fegan, N., Desmarchelier, P. & Benson, A. K. (2001) J Bacteriol 183, 6885-97.
51. Kumar, S., Tamura, K. & Nei, M. (2004) Brief Bioinform 5, 150-63.
52. Huson, D. H. (1998) Bioinformatics 14, 68-73.
53. Swaminathan, B., Barrett, T. J., Hunter, S. B. & Tauxe, R. V. (2001) Emerg Infect Dis 7, 382-9.
54. Ghai, R., Hain, T. & Chakraborty, T. (2004) BMC Bioinformatics 5, 198.
55. Zhang, W., Bielaszewska, M., Friedrich, A. W., Kuczius, T. & Karch, H. (2005) Appl Environ Microbiol 71, 558-61.
56. Riordan, J., Viswanath, S., Manning, S., Whittam, T. (2008) J of Clinical Microbiology 46, No. 6, 2070-2073.

What is claimed is:

1. A method for genotyping Escherichia coli O157:H7, comprising:
providing a sample of DNA from a possible E. coli O157:H7 infection;
detecting in the sample whether the identity of
the nucleotide at position 125 of SEQ ID NO. 11 is thymine (T) or guanine (G),
the nucleotide at position 648 of SEQ ID NO. 82 is T or cytosine (C),
the nucleotide at position 299 of SEQ ID NO. 47 is T or C,
the nucleotide at position 339 of SEQ ID NO. 15 is T or C,
the nucleotide at position 144 of SEQ ID NO. 67 is adenine (A) or G,
the nucleotide at position 417 of SEQ ID NO. 78 is T or C,
the nucleotide at position 3971 of SEQ ID NO. 52 is G or T,
the nucleotide at position 1186 of SEQ ID NO. 75 is C or G,
the nucleotide at position 2244 of SEQ ID NO. 81 is T or C,
the nucleotide at position 1151 of SEQ ID NO. 10 is T or C,
the nucleotide at position 1678 of SEQ ID NO. 16 is G or C,
the nucleotide at position 1545 of SEQ ID NO. 17 is G or A,
the nucleotide at position 311 of SEQ ID NO. 21 is G or A,
the nucleotide at position 1340 of SEQ ID NO. 48 is G or A,
the nucleotide at position 776 of SEQ ID NO. 35 is G or A,
the nucleotide at position 132 of SEQ ID NO. 57 is G or T,
the nucleotide at position 348 of SEQ ID NO. 46 is A or C,
the nucleotide at position 928 of SEQ ID NO. 20 is G or A,
the nucleotide at position 849 of SEQ ID NO. 36 is G or A,
the nucleotide at position 247 of SEQ ID NO. 79 is G or A,
the nucleotide at position 83 of SEQ ID NO. 1 is T or C,
the nucleotide at position 117 of SEQ ID NO. 6 is C or A,
the nucleotide at position 259 of SEQ ID NO. 22 is C or T,
the nucleotide at position 379 of SEQ ID NO. 18 is C or T,
the nucleotide at position 739 of SEQ ID NO. 4 is G or A,
the nucleotide at position 527 of SEQ ID NO. 47 is C or T,
the nucleotide at position 693 of SEQ ID NO. 74 is C or T,
the nucleotide at position 281 of SEQ ID NO. 11 is C or T,
the nucleotide at position 267 of SEQ ID NO. 57 is G or A,
the nucleotide at position 2707 of SEQ ID NO. 66 is C or A,
the nucleotide at position 354 of SEQ ID NO. 47 is C or A, and
the nucleotide at position 339 of SEQ ID NO. 70 is T or A; and
using the identities of these nucleotides to determine whether the possible E. coli O157:H7 has a single nucleotide polymorphism genotype (SG) of an E. coli O157:H7 that is defined by these nucleotides.

2. The method of claim 1, wherein the identity of
the nucleotide at position 125 of SEQ ID NO. 11 is G,
the nucleotide at position 648 of SEQ ID NO. 82 is C,
the nucleotide at position 299 of SEQ ID NO. 47 is C,
the nucleotide at position 339 of SEQ ID NO. 15 is C,
the nucleotide at position 144 of SEQ ID NO. 67 is G,
the nucleotide at position 417 of SEQ ID NO. 78 is C,
the nucleotide at position 3971 of SEQ ID NO. 52 is T,
the nucleotide at position 1186 of SEQ ID NO. 75 is G,
the nucleotide at position 2244 of SEQ ID NO. 81 is T,
the nucleotide at position 1151 of SEQ ID NO. 10 is C,
the nucleotide at position 1678 of SEQ ID NO. 16 is G,
the nucleotide at position 1545 of SEQ ID NO. 17 is G,
the nucleotide at position 311 of SEQ ID NO. 21 is G,
the nucleotide at position 1340 of SEQ ID NO. 48 is A,
the nucleotide at position 776 of SEQ ID NO. 35 is A,
the nucleotide at position 132 of SEQ ID NO. 57 is G,
the nucleotide at position 348 of SEQ ID NO. 46 is A,
the nucleotide at position 928 of SEQ ID NO. 20 is G,
the nucleotide at position 849 of SEQ ID NO. 36 is G,
the nucleotide at position 247 of SEQ ID NO. 79 is G,
the nucleotide at position 83 of SEQ ID NO. 1 is C,
the nucleotide at position 117 of SEQ ID NO. 6 is C,
the nucleotide at position 259 of SEQ ID NO. 22 is C or T,
the nucleotide at position 379 of SEQ ID NO. 18 is C or T,
the nucleotide at position 739 of SEQ ID NO. 4 is G or A,
the nucleotide at position 527 of SEQ ID NO. 47 is C or T,
the nucleotide at position 693 of SEQ ID NO. 74 is C or T,
the nucleotide at position 281 of SEQ ID NO. 11 is T,
the nucleotide at position 267 of SEQ ID NO. 57 is G,
the nucleotide at position 2707 of SEQ ID NO. 66 is C,
the nucleotide at position 354 of SEQ ID NO. 47 is C, and
the nucleotide at position 339 of SEQ ID NO. 70 is T; and
the possible E. coli O157:H7 is determined to have a SG of an E. coli O157:H7 clade associated with more severe disease.

3. The method of claim 1, wherein the SG determination identifies the genotype of E. coli O157:H7.

4. The method of claim 1, wherein the SG identifies the clade of E. coli O157:H7.

5. The method of claim 1, wherein the SG determination is used to diagnose infection by E. coli O157:H7.

6. The method of claim 1, wherein the sample is from a plant or animal.

7. The method of claim 6, wherein the sample is from an animal.

8. The method of claim 7, wherein the animal is a human.

9. The method of claim 1, wherein the detecting is by a real-time polymerase chain reaction (PCR) assay.

10. The method of claim 9, wherein at least one primer trio is used to detect the identity of a nucleotide in the PCR assay.

11. The method of claim 10, wherein the primer trio is selected from the group consisting of SEQ ID NOs. 83-382.

12. The method of claim 1, wherein the SG is one of thirty-nine SGs defined by these nucleotides.

13. The method of claim 1, wherein the SG is one of thirty-six SGs defined by these nucleotides.

14. The method of claim 1, wherein the SG is one of thirty-three SGs defined by these nucleotides.

15. A kit comprising at least three primers selected from the group consisting of oligonucleotides identified by SEQ ID NOs. 83-382.
